(19) United States
(12) Patent Application Publication
(10) Pub. No.: US 2006/0005281 A1
(43) Pub. Date: Jan. 5, 2006

(54) Production of plants having improved rooting efficiency and vase life using stress-resistance gene

(75) Inventors: Kazuko Shinozaki, Ibaraki (JP); Naoyuki Umemoto, Tochigi (JP); Kanji Mamiya, Tochigi (JP); Toshihiro Toguri, Tochigi (JP)

Correspondence Address: FOLEY AND LARDNER LLP;SUITE 500, 3000 K STREET NW, WASHINGTON, DC, 20007, US

(73) Assignees: Japan International Research Center for Agricultural Science; Kirin Beer Kabushiki Kaisha

(21) Appl. No.: 10/798,579

(22) Filed: Mar. 12, 2004

(30) Foreign Application Priority Data
Mar. 14, 2003 (JP) 2003-71082

(51) Int. Cl. A01H 1/00; C12N 15/82

(52) U.S. Cl. 800/287; 435/468

(57) ABSTRACT

Provided is a plant having improved efficiency in propagation by cutting resulting from the enhanced rooting efficiency, and improved vase life. A method of producing a transformed plant having improved rooting efficiency and/or prolonged vase life, comprising transforming a plant using a gene wherein a DNA encoding a protein that binds to a stress-responsive element contained in a stress-responsive promoter and regulates the transcription of a gene located downstream of the element is ligated downstream of the stress-responsive promoter.

### BACKGROUND OF THE INVENTION

1. Field of the Invention

The present invention relates to a method of producing a transformed plant having improved rooting efficiency and/or prolonged vase life, which comprises transforming a plant with a gene wherein a DNA encoding a protein that binds to a stress responsive element contained in a stress-responsive promoter and regulates the transcription of a gene located downstream of the element is ligated downstream of the stress-responsive promoter, and relates to a transformed plant having improved rooting efficiency and/or prolonged vase life, which comprises a gene wherein a DNA encoding a protein that binds to a stress responsive element contained in a stress-responsive promoter so as to regulate the transcription of a gene located downstream of the element is ligated downstream of the stress-responsive promoter.

The present invention further relates to the use of a gene (stress-resistance gene) wherein a DNA encoding a protein that binds to a dehydration responsive element (DRE) for production of a plant having improved rooting efficiency and/or prolonged vase life so as to regulate the transcription of a gene located downstream of the DRE is ligated downstream of the stress-responsive promoter.

2. Description of Related Art

Cultivated plants are grown by natural plant mechanisms such as breeding by seeds and bulbs, and by cloning technique such as cutting (herbaceous cutting and scion) and tissue culture. Particularly, concerning 3 important cut flowers, chrysanthemum, carnation, and rose, when a good variety is produced, its branches and buds are propagated by cutting (herbaceous cutting and scion), and the propagated plants are used for the production of cut flowers and the maintenance of the plant variety. To raise the productivity of this variety, the propagation efficiency of cutting should be raised. To raise the productivity to the highest, the rooting ability of cutting should be improved. To address the problem, treatment with a chemical such as auxins represented by the trade name of Rooton or the like has been conducted. However, it is never sufficient, it costs much, and it takes time under current conditions. In the meantime, it goes without saying that the property of keeping the quality of flowers (prolonged vase life) is a very important character of cut flowers. Biochemical examinations regarding vase life have been conducted, so that, for example, a technique of physically absorbing ethylene, which is an aging hormone, has been developed. However, in this method, the vase life controlled by ethylene does not represent a substantial improvement with regard to cut flowers, but rather only a partial improvement. Furthermore, the varieties of plants that can be improved by absorption of ethylene or suppression of ethylene generation are limited, so that improvement in applicability to more various plant varieties and in plants' own conditions has been expected. Besides, there has been no known means for improving rooting ability and prolonging the vase life of cut flowers at the same time.

To date, in the case of artificially producing a plant having improved propagation ability in terms of clonal productivity or vase life, techniques such as selection and crossing of lines showing excellent characters relating to each of these properties have been employed. However, while the selection method requires long term, the crossing method can be used only between related species. Thus, it has been difficult to produce plants having improved propagation efficiency with reference to cutting and improved vase life.

With the progress of biotechnology in recent years, the production of various plants has been attempted using techniques such as transformation technology, whereby a specific gene derived from an organism of a different species is introduced into a plant. To date, regarding the promotion of rooting, there has been a case involving the introduction of a rolC gene into a carnation. However, since the rolC gene itself has been known to promote dwarfing or to enhance branching in a various plants, the practical application thereof may be difficult [J. Amer. Soc. Hort. Sci. 126: 13-18 (2001)]. Suppressing the generation of ethylene or making the ethylene-receptive mechanism insensitive has been attempted by genetic modification. It has been reported that the produced plant so far could have partially improved vase life (suppressed aging of flower petals and the like) [HortScience 30: 970-972 (1995); Mol. Breed. 5: 301-308 (1999)].

In the meantime, plants inhabit naturally exposing themselves to various environmental stresses such as drought, high temperature, low temperature, or salinity. Since plants are unable to take action to protect themselves from stress by moving as animals do, they have acquired various stress-resistance mechanisms in the process of evolution. For example, low-temperature-resistant plants (e.g., Arabidopsis, spinach, lettuce, pea, barley, and beet) have a lower content of unsaturated fatty acid in biological membrane lipids compared with the case of low-temperature-sensitive plants (e.g., corn, rice, pumpkin, cucumber, banana, and tomato), so that when the low-temperature-resistant plants are exposed to low temperature, the phase transition of biological membrane lipids occurs with difficulty and thus low temperature injuries are not easily caused. To date, in the case of artificially producing environmental stress-resistant plants, techniques such as selection and crossing of lines with resistance against drought, low temperatures, or salinity have been employed. However, while the selection method requires long term, the crossing method can be used only between related species. Thus, it has been difficult to produce plants having strong resistance against various environmental stress.

With the progress of biotechnology in recent years, the production of plants resistant to drought, low temperature, salinity, or the like has been attempted using techniques such as transformation technology, whereby a specific gene derived from an organism of a different species is introduced into a plant. An example of a plant that is thought to be the most practical use is an environmental-stress-resistant transformed plant [JP Patent Nos. 3178672 and 3183458] that has been produced by introducing a gene, wherein a DNA (referred to as DREB gene) encoding a transcription factor having functions to bind to dehydration responsive element (DRE) so as to activate the transcription of a gene located downstream of DRE is ligated downstream of a stress-responsive promoter. By the use of this method, transformed plants having improved resistance against forms of environmental stresses (e.g., drought stress, low temperature stress, and salinity stress) and exhibiting no dwarfing are generated. However, such stress resistance has been conferred when plants are assumed to be cultivated under special conditions (e.g., cultivated continuously in desert areas, areas damaged by salinity, and low temperature areas), or when plants are exposed temporarily to extreme forms of environmental stress. It has not been reported that the thus-conferred resistance against stress has a favorable effect on the rooting efficiency for propagation by cutting, the ordinary form of cultivation, the vase life of cut flowers (prolonged life of cut flowers), the ordinary form of the distribution of products, or that of consumption.

### SUMMARY OF THE INVENTION

An object of the present invention is to provide plants having better propagation efficiency by cutting that is improved by increasing rooting efficiency, and having improved vase life.

We have conducted experiments as a result of intensive studies to achieve the above object. We have completed the present invention by obtaining chrysanthemum transformed with a plant transformation plasmid pBI29AP:DREB1A (produced for the purpose of conferring stress resistance as disclosed in Example 5 of Japanese Patent No. 3178672), propagating the plant by cloning technique, producing cut flowers from the plant, examining the vase life of this plant, and comparing the chrysanthemum without gene transformation. We found clear superiority of this transformed plant in rooting efficiency, propagation ability by cutting, and vase life (prolonged life of cut flowers). That is, the present invention is as follows

(1) A method of producing a transformed plant having improved rooting efficiency and/or prolonged vase life, comprising transforming a plant using a gene wherein a DNA encoding a protein that binds to a stress-responsive element contained in a stress-responsive promoter and regulates the transcription of a gene located downstream of the element is ligated downstream of the stress-responsive promoter.

(2) The method of producing a transformed plant of (1), wherein the stress-responsive promoter is at least one promoter selected from the group consisting of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter.

(3) The method of producing a transformed plant of (1), wherein the DNA encoding a protein that binds to a stress-responsive element and regulates the transcription of a gene located downstream of the element is at least one gene selected from the group consisting of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene.

(4) The method of producing a transformed plant of (1), wherein the DNA encoding a protein that binds to a stress-responsive element and regulates the transcription of a gene located downstream of the element is at least one DNA selected from the group consisting of:
(a) a DNA comprising a nucleotide sequence derived from the nucleotide sequence of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene by deletion, substitution, addition, or insertion of one or several nucleotides, and encoding a protein having activity to bind to a stress-responsive element and regulate the transcription of a gene located downstream of the element;
(b) a DNA comprising a nucleotide sequence having at least 80% or more homology with the nucleotide sequence of a DNA of at least one of DREB 1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element and regulate the transcription of a gene located downstream of the element; and
(c) a DNA hybridizing under stringent conditions to a DNA complementary to a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element and regulate the transcription of a gene located downstream of the element.

(5) The method of producing a transformed plant of (1), wherein the DNA of a stress-responsive promoter is at least one DNA selected from the group consisting of:
(a) a DNA comprising a nucleotide sequence derived from the nucleotide sequence of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter by deletion, substitution, addition, or insertion of one or several nucleotides, and having activity as the DNA of the stress-responsive promoter;
(b) a DNA comprising a nucleotide sequence having at least 80% or more homology with the nucleotide sequence of at least one DNA of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter, and having activity as the DNA of the stress-responsive promoter; and
(c) a DNA hybridizing under stringent conditions to a DNA complementary to a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter, and having activity as the DNA of the stress-responsive promoter.

(6) A transformed plant having improved rooting efficiency and/or prolonged vase life, comprising a gene wherein a DNA encoding a protein that binds to a stress-responsive element contained in a stress-responsive promoter and regulates the transcription of a gene located downstream of the element is ligated downstream of the stress-responsive promoter.

(7) The transformed plant of (6), wherein the stress-responsive promoter is at least one promoter selected from the group consisting of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter.

(8) The transformed plant of (6), wherein the DNA encoding a protein that binds to a stress-responsive element so as to regulate the transcription of a gene located downstream of the element is at least one gene selected from the group consisting of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene.

(9) The transformed plant of (6), wherein the DNA encoding a protein that binds to a stress-responsive element so as to regulate the transcription of a gene located downstream of the element is at least one DNA selected from the group consisting of:
(a) a DNA comprising a nucleotide sequence derived from the nucleotide sequence of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene by deletion, substitution, addition, or insertion of one or several nucleotides, and encoding a protein having activity to bind to a stress-responsive element so as to regulate the transcription of a gene located downstream of the element;
(b) a DNA comprising a nucleotide sequence having at least 80% or more homology with the nucleotide sequence of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element so as to regulate the transcription of a gene located downstream of the element; and
(c) a DNA hybridizing under stringent conditions to a DNA complementary to a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element so as to regulate the transcription of a gene located downstream of the element.

(10) The transformed plant of (6), wherein the DNA of a stress-responsive promoter is at least one DNA selected from the group consisting of:
(a) a DNA comprising a nucleotide sequence derived from the nucleotide sequence of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter by deletion, substitution, addition, or insertion of one or several nucleotides, and having activity as the DNA of the stress-responsive promoter;
(b) a DNA comprising a nucleotide sequence having at least 80% or more homology with the nucleotide sequence of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter, and having activity as the DNA of the stress-responsive promoter; and
(c) a DNA hybridizing under stringent conditions to a DNA complementary to a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter, and having activity as the DNA of the stress-responsive promoter.

Furthermore, the DNAs of (4) and (9) above include a DNA having activity substantially equivalent to that of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene. The DNAs of (5) and (10) include a DNA having activity substantially equivalent to that of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter.

### BRIEF DESCRIPTION OF THE DRAWINGS

FIG. 1 shows the structure between RB and LB of a rd29A-DREB1A vector.

FIG. 2-1 shows 1 to 1 alignment, common sequences and homology % at the nucleotide sequence level between DREB1A as a standard and each one of DREB1B to DREB1F.

FIG. 2-2 shows 1 to 1 alignment, common sequences and homology % at the nucleotide sequence level between DREB1A as a standard and each one of DREB1B to DREB1F (continued from FIG. 2-1).

FIG. 2-3 shows 1 to 1 alignment, common sequences and homology % at the nucleotide sequence level between DREB1A as a standard and each one of DREB1B to DREB1F (continued from FIG. 2-2).

FIG. 2-4 shows 1 to 1 alignment, common sequences and homology % at the nucleotide sequence level between DREB1A as a standard and each one of DREB1B to DREB1F (continued from FIG. 2-3).

FIG. 2-5 shows 1 to 1 alignment, common sequences and homology % at the nucleotide sequence level between DREB1A as a standard and each one of DREB1B to DREB1F (continued from FIG. 2-4).

FIG. 2-6 shows 1 to 1 alignment, common sequences and homology % at the nucleotide sequence level between DREB1A as a standard and each one of DREB1B to DREB1F (continued from FIG. 2-5).

FIG. 2-7 shows 1 to 1 alignment, common sequences and homology % at the nucleotide sequence level between DREB1A as a standard and each one of DREB1B to DREB1F (continued from FIG. 2-6).

FIG. 2-8 shows 1 to 1 alignment, common sequences and homology % at the nucleotide sequence level between DREB1A as a standard and each one of DREB1B to DREB1F (continued from FIG. 2-7).

FIG. 2-9 shows 1 to 1 alignment, common sequences and homology % at the nucleotide sequence level between DREB1A as a standard and each one of DREB1B to DREB1F (continued from FIG. 2-8).

FIG. 2-10 shows 1 to 1 alignment, common sequences and homology % at the nucleotide sequence level between DREB1A as a standard and each one of DREB1B to DREB1F (continued from FIG. 2-9).

FIG. 2-11 shows 1 to 1 alignment, common sequences and homology % at the nucleotide sequence level between DREB1A as a standard and each one of DREB1B to DREB1F (continued from FIG. 2-10).

FIG. 2-12 shows 1 to 1 alignment, common sequences and homology % at the nucleotide sequence level between DREB1A as a standard and each one of DREB1B to DREB1F (continued from FIG. 2-11).

FIG. 2-13 shows 1 to 1 alignment, common sequences and homology % at the nucleotide sequence level between DREB1A as a standard and each one of DREB1B to DREB1F (continued from FIG. 2-12).

FIG. 2-14 shows 1 to 1 alignment, common sequences and homology % at the nucleotide sequence level between DREB1A as a standard and each one of DREB1B to DREB1F (continued from FIG. 2-13).

FIG. 2-15 shows 1 to 1 alignment, common sequences and homology % at the nucleotide sequence level between DREB1A as a standard and each one of DREB1B to DREB1F (continued from FIG. 2-14).

FIG. 2-16 shows 1 to 1 alignment, common sequences and homology % at the nucleotide sequence level between DREB1A as a standard and each one of DREB1B to DREB1F (continued from FIG. 2-15).

FIG. 3-1 shows 1 to 1 alignment, common sequences and homology % at the amino acid sequence level between DREB1A as a standard and each one of DREB1B to DREB1F.

FIG. 3-2 shows 1 to 1 alignment, common sequences and homology % at the amino acid sequence level between DREB1A as a standard and each one of DREB1B to DREB1F (continued from FIG. 3-1).

FIG. 3-3 shows 1 to 1 alignment, common sequences and homology % at the amino acid sequence level between DREB1A as a standard and each one of DREB1B to DREB1F (continued from FIG. 3-2).

FIG. 3-4 shows 1 to 1 alignment, common sequences and homology % at the amino acid sequence level between DREB1A as a standard and each one of DREB1B to DREB1F (continued from FIG. 3-3).

FIG. 3-5 shows 1 to 1 alignment, common sequences and homology % at the amino acid sequence level between DREB1A as a standard and each one of DREB1B to DREB1F (continued from FIG. 3-4).

FIG. 3-6 shows 1 to 1 alignment, common sequences and homology % at the amino acid sequence level between DREB1A as a standard and each one of DREB1B to DREB1F (continued from FIG. 3-5).

FIG. 3-7 shows 1 to 1 alignment, common sequences and homology % at the amino acid sequence level between DREB1A as a standard and each one of DREB1B to DREB1F (continued from FIG. 3-6).

FIG. 3-8 shows 1 to 1 alignment, common sequences and homology % at the amino acid sequence level between DREB1A as a standard and each one of DREB1B to DREB1F (continued from FIG. 3-7).

FIG. 3-9 shows 1 to 1 alignment, common sequences and homology % at the amino acid sequence level between DREB1A as a standard and each one of DREB1B to DREB1F (continued from FIG. 3-8).

FIG. 4-1 shows 1 to 1 alignment, common sequences and homology % at the nucleotide sequence level between DREB2A as a standard and each one of DREB2B to DREB2H.

FIG. 4-2 shows 1 to 1 alignment, common sequences and homology % at the nucleotide sequence level between DREB2A as a standard and each one of DREB2B to DREB2H (continued from FIG. 4-1).

FIG. 4-3 shows 1 to 1 alignment, common sequences and homology % at the nucleotide sequence level between DREB2A as a standard and each one of DREB2B to DREB2H (continued from FIG. 4-2).

FIG. 4-4 shows 1 to 1 alignment, common sequences and homology % at the nucleotide sequence level between DREB2A as a standard and each one of DREB2B to DREB2H (continued from FIG. 4-3).

FIG. 4-5 shows 1 to 1 alignment, common sequences and homology % at the nucleotide sequence level between DREB2A as a standard and each one of DREB2B to DREB2H (continued from FIG. 4-4).

FIG. 4-6 shows 1 to 1 alignment, common sequences and homology % at the nucleotide sequence level between DREB2A as a standard and each one of DREB2B to DREB2H (continued from FIG. 4-5).

FIG. 4-7 shows 1 to 1 alignment, common sequences and homology % at the nucleotide sequence level between DREB2A as a standard and each one of DREB2B to DREB2H (continued from FIG. 4-6).

FIG. 4-8 shows 1 to 1 alignment, common sequences and homology % at the nucleotide sequence level between DREB2A as a standard and each one of DREB2B to DREB2H (continued from FIG. 4-7).

FIG. 4-9 shows 1 to 1 alignment, common sequences and homology % at the nucleotide sequence level between DREB2A as a standard and each one of DREB2B to DREB2H (continued from FIG. 4-8).

FIG. 4-10 shows 1 to 1 alignment, common sequences and homology % at the nucleotide sequence level between DREB2A as a standard and each one of DREB2B to DREB2H (continued from FIG. 4-9).

FIG. 4-11 shows 1 to 1 alignment, common sequences and homology % at the nucleotide sequence level between DREB2A as a standard and each one of DREB2B to DREB2H (continued from FIG. 4-10).

FIG. 4-12 shows 1 to 1 alignment, common sequences and homology % at the nucleotide sequence level between DREB2A as a standard and each one of DREB2B to DREB2H (continued from FIG. 4-11).

FIG. 4-13 shows 1 to 1 alignment, common sequences and homology % at the nucleotide sequence level between DREB2A as a standard and each one of DREB2B to DREB2H (continued from FIG. 4-12).

FIG. 4-14 shows 1 to 1 alignment, common sequences and homology % at the nucleotide sequence level between DREB2A as a standard and each one of DREB2B to DREB2H (continued from FIG. 4-13).

FIG. 4-15 shows 1 to 1 alignment, common sequences and homology % at the nucleotide sequence level between DREB2A as a standard and each one of DREB2B to DREB2H (continued from FIG. 4-14).

FIG. 4-16 shows 1 to 1 alignment, common sequences and homology % at the nucleotide sequence level between DREB2A as a standard and each one of DREB2B to DREB2H (continued from FIG. 4-15).

FIG. 4-17 shows 1 to 1 alignment, common sequences and homology % at the nucleotide sequence level between DREB2A as a standard and each one of DREB2B to DREB2H (continued from FIG. 4-16).

FIG. 4-18 shows 1 to 1 alignment, common sequences and homology % at the nucleotide sequence level between DREB2A as a standard and each one of DREB2B to DREB2H (continued from FIG. 4-17).

FIG. 4-19 shows 1 to 1 alignment, common sequences and homology % at the nucleotide sequence level between DREB2A as a standard and each one of DREB2B to DREB2H (continued from FIG. 4-18).

FIG. 4-20 shows 1 to 1 alignment, common sequences and homology % at the nucleotide sequence level between DREB2A as a standard and each one of DREB2B to DREB2H (continued from FIG. 4-19).

FIG. 4-21 shows 1 to 1 alignment, common sequences and homology % at the nucleotide sequence level between DREB2A as a standard and each one of DREB2B to DREB2H (continued from FIG. 4-20).

FIG. 4-22 shows 1 to 1 alignment, common sequences and homology % at the nucleotide sequence level between DREB2A as a standard and each one of DREB2B to DREB2H (continued from FIG. 4-21).

FIG. 4-23 shows 1 to 1 alignment, common sequences and homology % at the nucleotide sequence level between DREB2A as a standard and each one of DREB2B to DREB2H (continued from FIG. 4-22).

FIG. 4-24 shows 1 to 1 alignment, common sequences and homology % at the nucleotide sequence level between DREB2A as a standard and each one of DREB2B to DREB2H (continued from FIG. 4-23).

FIG. 4-25 shows 1 to 1 alignment, common sequences and homology % at the nucleotide sequence level between DREB2A as a standard and each one of DREB2B to DREB2H (continued from FIG. 4-24).

FIG. 4-26 shows 1 to 1 alignment, common sequences and homology % at the nucleotide sequence level between DREB2A as a standard and each one of DREB2B to DREB2H (continued from FIG. 4-25).

FIG. 4-27 shows 1 to 1 alignment, common sequences and homology % at the nucleotide sequence level between DREB2A as a standard and each one of DREB2B to DREB2H (continued from FIG. 4-26).

FIG. 4-28 shows 1 to 1 alignment, common sequences and homology % at the nucleotide sequence level between DREB2A as a standard and each one of DREB2B to DREB2H (continued from FIG. 4-27).

FIG. 4-29 shows 1 to 1 alignment, common sequences and homology % at the nucleotide sequence level between DREB2A as a standard and each one of DREB2B to DREB2H (continued from FIG. 4-28).

FIG. 4-30 shows 1 to 1 alignment, common sequences and homology % at the nucleotide sequence level between DREB2A as a standard and each one of DREB2B to DREB2H (continued from FIG. 4-29).

FIG. 4-31 shows 1 to 1 alignment, common sequences and homology % at the nucleotide sequence level between DREB2A as a standard and each one of DREB2B to DREB2H (continued from FIG. 4-30).

FIG. 4-32 shows 1 to 1 alignment, common sequences and homology % at the nucleotide sequence level between DREB2A as a standard and each one of DREB2B to DREB2H (continued from FIG. 4-31).

FIG. 4-33 shows 1 to 1 alignment, common sequences and homology % at the nucleotide sequence level between DREB2A as a standard and each one of DREB2B to DREB2H (continued from FIG. 4-32).

FIG. 4-34 shows 1 to 1 alignment, common sequences and homology % at the nucleotide sequence level between DREB2A as a standard and each one of DREB2B to DREB2H (continued from FIG. 4-33).

FIG. 4-35 shows 1 to 1 alignment, common sequences and homology % at the nucleotide sequence level between DREB2A as a standard and each one of DREB2B to DREB2H (continued from FIG. 4-34).

FIG. 4-36 shows 1 to 1 alignment, common sequences and homology % at the nucleotide sequence level between DREB2A as a standard and each one of DREB2B to DREB2H (continued from FIG. 4-35).

FIG. 4-37 shows 1 to 1 alignment, common sequences and homology % at the nucleotide sequence level between DREB2A as a standard and each one of DREB2B to DREB2H (continued from FIG. 4-36).

FIG. 4-38 shows 1 to 1 alignment, common sequences and homology % at the nucleotide sequence level between DREB2A as a standard and each one of DREB2B to DREB2H (continued from FIG. 4-37).

FIG. 4-39 shows 1 to 1 alignment, common sequences and homology % at the nucleotide sequence level between DREB2A as a standard and each one of DREB2B to DREB2H (continued from FIG. 4-38).

FIG. 4-40 shows 1 to 1 alignment, common sequences and homology % at the nucleotide sequence level between DREB2A as a standard and each one of DREB2B to DREB2H (continued from FIG. 4-39).

FIG. 5-1 shows 1 to 1 alignment, common sequences and homology % at the amino acid sequence level between DREB2A as a standard and each one of DREB2B to DREB2H.

FIG. 5-2 shows 1 to 1 alignment, common sequences and homology % at the amino acid sequence level between DREB2A as a standard and each one of DREB2B to DREB2H (continued from FIG. 5-1).

FIG. 5-3 shows 1 to 1 alignment, common sequences and homology % at the amino acid sequence level between DREB2A as a standard and each one of DREB2B to DREB2H (continued from FIG. 5-2).

FIG. 5-4 shows 1 to 1 alignment, common sequences and homology % at the amino acid sequence level between DREB2A as a standard and each one of DREB2B to DREB2H (continued from FIG. 5-3).

FIG. 5-5 shows 1 to 1 alignment, common sequences and homology % at the amino acid sequence level between DREB2A as a standard and each one of DREB2B to DREB2H (continued from FIG. 5-4).

FIG. 5-6 shows 1 to 1 alignment, common sequences and homology % at the amino acid sequence level between DREB2A as a standard and each one of DREB2B to DREB2H (continued from FIG. 5-5).

FIG. 5-7 shows 1 to 1 alignment, common sequences and homology % at the amino acid sequence level between DREB2A as a standard and each one of DREB2B to DREB2H (continued from FIG. 5-6).

FIG. 5-8 shows 1 to 1 alignment, common sequences and homology % at the amino acid sequence level between DREB2A as a standard and each one of DREB2B to DREB2H (continued from FIG. 5-7).

FIG. 5-9 shows 1 to 1 alignment, common sequences and homology % at the amino acid sequence level between DREB2A as a standard and each one of DREB2B to DREB2H (continued from FIG. 5-8).

FIG. 5-10 shows 1 to 1 alignment, common sequences and homology % at the amino acid sequence level between DREB2A as a standard and each one of DREB2B to DREB2H (continued from FIG. 5-9).

FIG. 5-11 shows 1 to 1 alignment, common sequences and homology % at the amino acid sequence level between DREB2A as a standard and each one of DREB2B to DREB2H (continued from FIG. 5-10).

FIG. 5-12 shows 1 to 1 alignment, common sequences and homology % at the amino acid sequence level between DREB2A as a standard and each one of DREB2B to DREB2H (continued from FIG. 5-11).

FIG. 5-13 shows 1 to 1 alignment, common sequences and homology % at the amino acid sequence level between DREB2A as a standard and each one of DREB2B to DREB2H (continued from FIG. 5-12).

FIG. 5-14 shows 1 to 1 alignment, common sequences and homology % at the amino acid sequence level between DREB2A as a standard and each one of DREB2B to DREB2H (continued from FIG. 5-13).

FIG. 5-15 shows 1 to 1 alignment, common sequences and homology % at the amino acid sequence level between DREB2A as a standard and each one of DREB2B to DREB2H (continued from FIG. 5-14).

FIG. 5-16 shows 1 to 1 alignment, common sequences and homology % at the amino acid sequence level between DREB2A as a standard and each one of DREB2B to DREB2H (continued from FIG. 5-15).

FIG. 5-17 shows 1 to 1 alignment, common sequences and homology % at the amino acid sequence level between DREB2A as a standard and each one of DREB2B to DREB2H (continued from FIG. 5-16).

FIG. 5-18 shows 1 to 1 alignment, common sequences and homology % at the amino acid sequence level between DREB2A as a standard and each one of DREB2B to DREB2H (continued from FIG. 5-17).

FIG. 5-19 shows 1 to 1 alignment, common sequences and homology % at the amino acid sequence level between DREB2A as a standard and each one of DREB2B to DREB2H (continued from FIG. 5-18).

FIG. 5-20 shows 1 to 1 alignment, common sequences and homology % at the amino acid sequence level between DREB2A as a standard and each one of DREB2B to DREB2H (continued from FIG. 5-19).

FIG. 6 shows alignment at the nucleotide sequence level between DREB1A as a standard and each one of DREB1B to DREB1F.

FIG. 7-1 shows alignment at the nucleotide sequence level between DREB2A as a standard and each one of DREB2B to DREB2H (to position 518 of DREB2A).

FIG. 7-2 shows alignment at the nucleotide sequence level between DREB2A as a standard and each one of DREB2B to DREB2H (from position 519 of DREB2A).

FIG. 8 shows alignment at the amino acid sequence level between DREB1A as a standard and each one of DREB1B to DREB1F.

FIG. 9 shows alignment at the amino acid sequence level between DREB2A as a standard and each one of DREB2B to DREB2H.

FIG. 10 shows photographs showing the rooting ability of non-transformants, and lines 9 and 10 in the rooting ability test upon production with scions.

FIG. 11 is a graph showing the stem lengths of non-transformants, and lines 9 and 10 after planting.

FIG. 12 shows photographs showing the vicinity of the cut ends of non-transformants, and lines 9 and 10 on day 22 after the start of a vase life test.

### DETAILED DESCRIPTION OF THE INVENTION

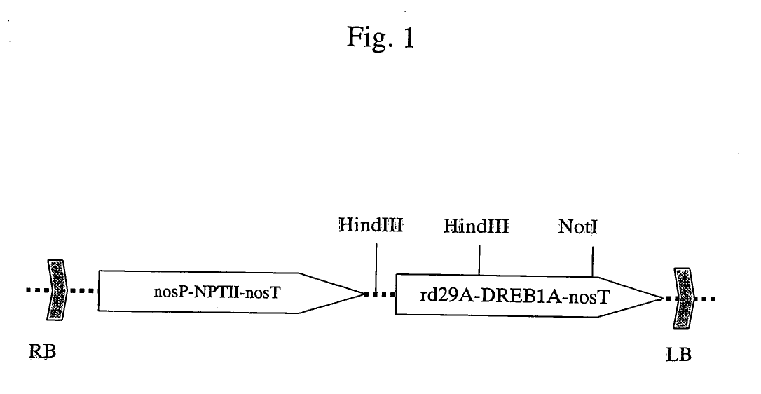

The transformed plant of the present invention is produced by introducing a gene (also referred to as a stress-resistance gene in this specification) wherein a DNA (also referred to as DREB gene) encoding a transcription factor that has functions to bind to a dehydration responsive element (DRE) contained in a stress-responsive promoter and activate the transcription of a gene located downstream of the DRE is ligated downstream of the stress-responsive promoter. The transformed plant has enhanced efficiency of propagation by cutting as a result of improving the rooting efficiency, and has improved vase life (prolonged life of cut flowers). As an example, a gene having a structure wherein the rd29A promoter is used is shown (FIG. 1).

(1) DREB Gene

Examples of the DNA of the present invention encoding a transcription factor having functions to bind to a dehydration responsive element (DRE) and activate the transcription of a gene located downstream of the DRE include DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene and DREB2H gene, and they can be used as appropriate. The DREB1A gene can be obtained by amplifying the cDNA region of the DREB1A gene [Kazuko Yamaguchi-Shinozaki and Kazuo Shinozaki: Plant Cell 6: 251-264 (1994)] by performing a reverse transcription polymerase chain reaction (also referred to as RT-PCR). An example of a template mRNA that can be used for PCR herein is mRNA that is prepared from Arabidopsis plants inoculated and grown on solid media such as MS media [Murashige and Skoog: Physiol. Plant. 15: 473-497 (1962)] under aseptic conditions, and then exposed to dehydration stress (e.g., putting the plants under drought).

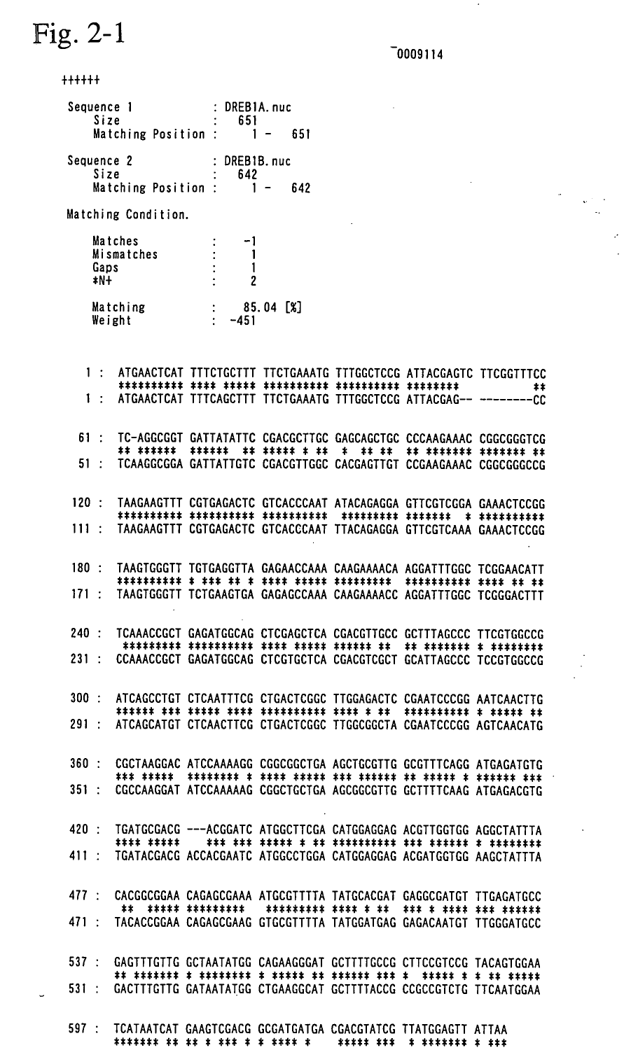

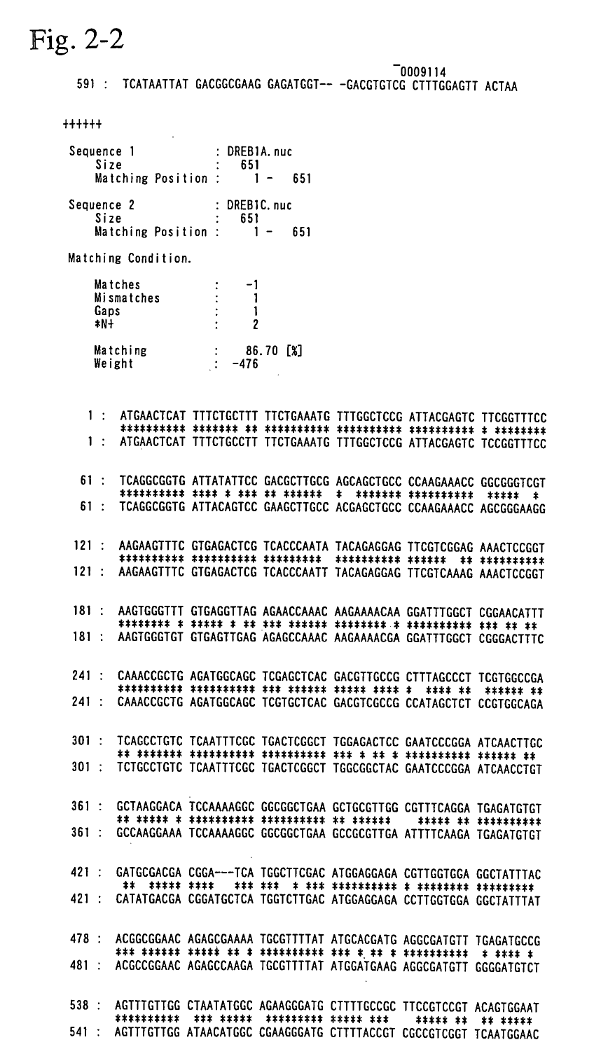

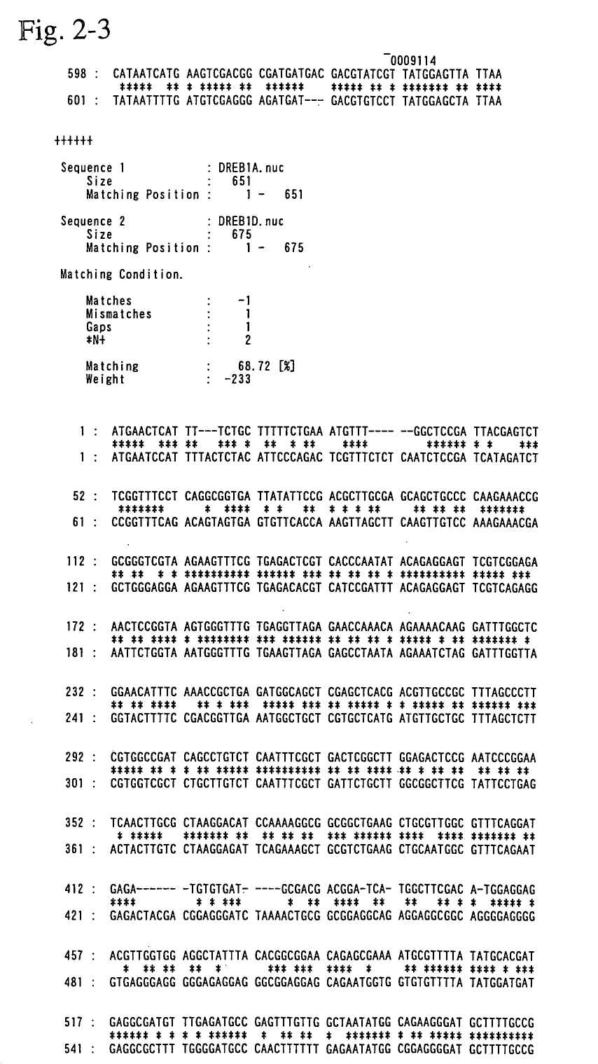

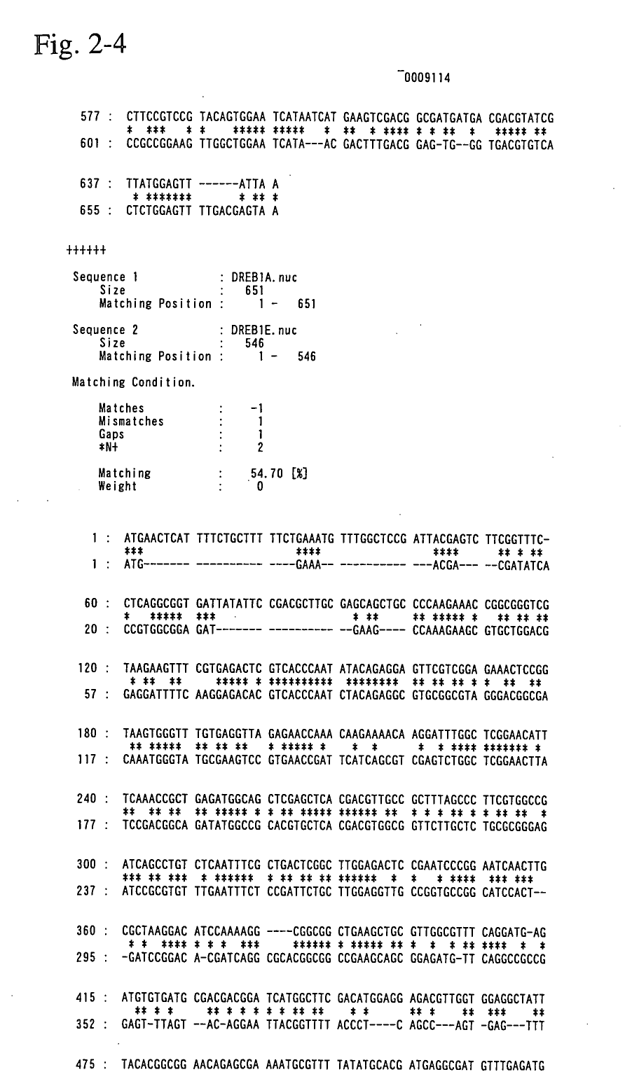

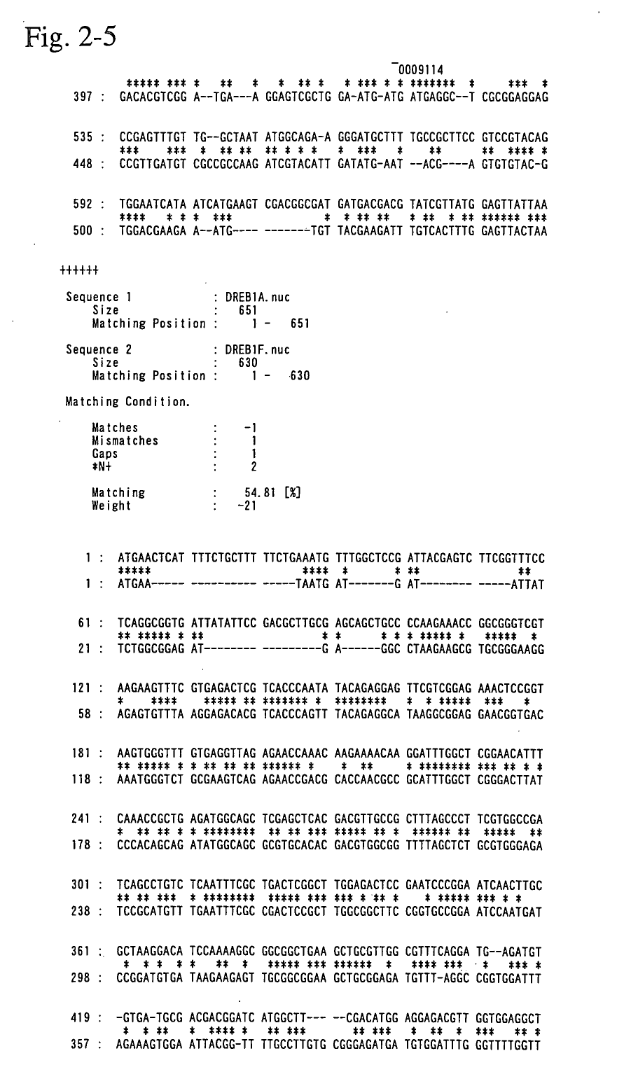

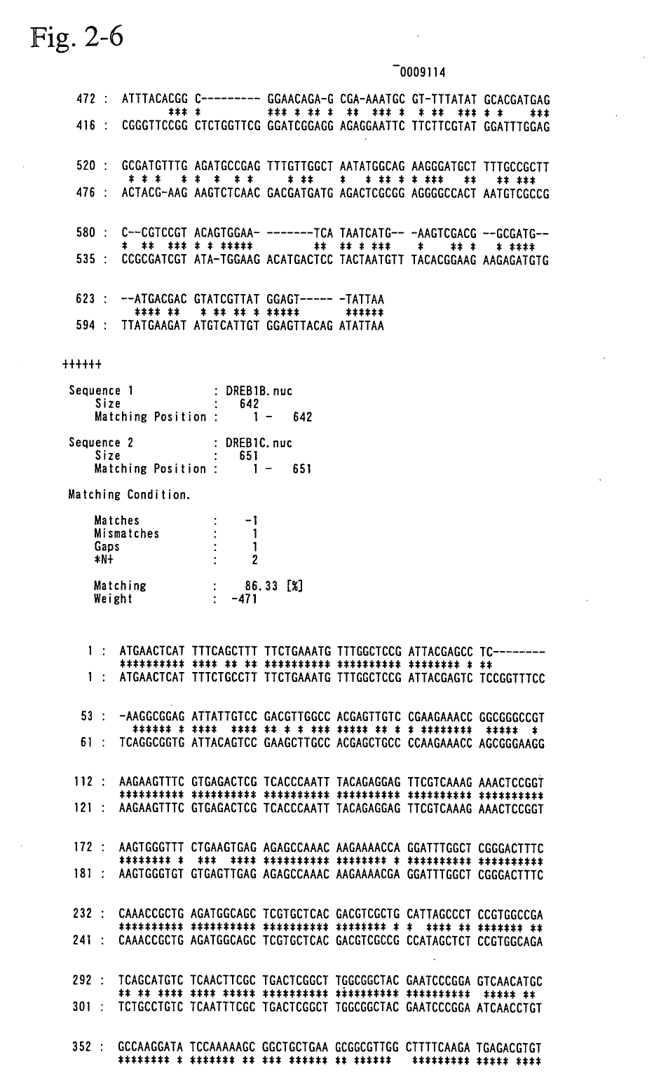

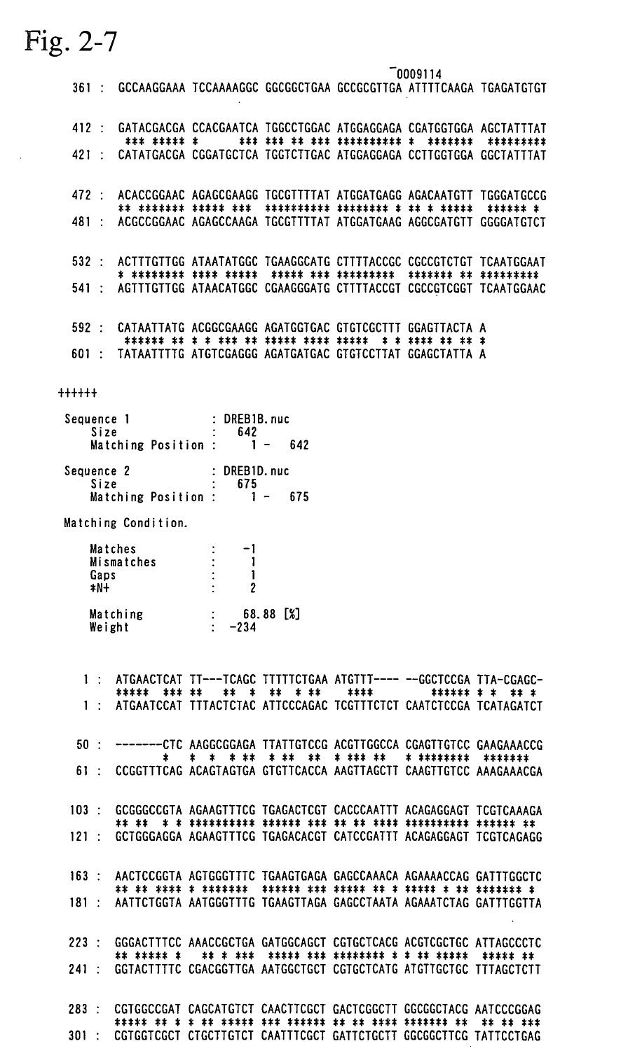

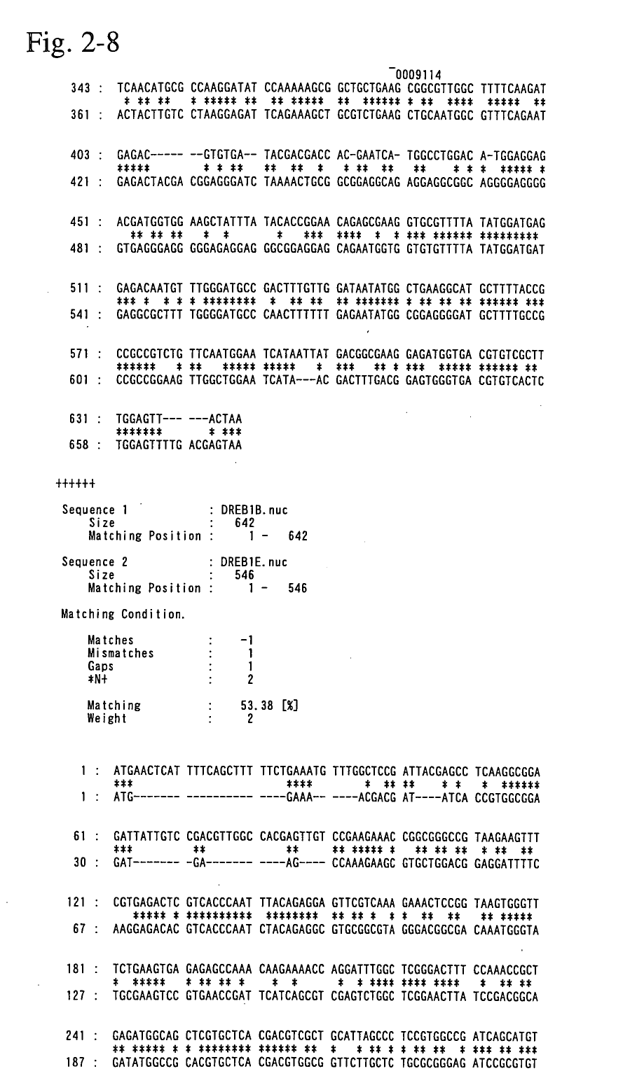

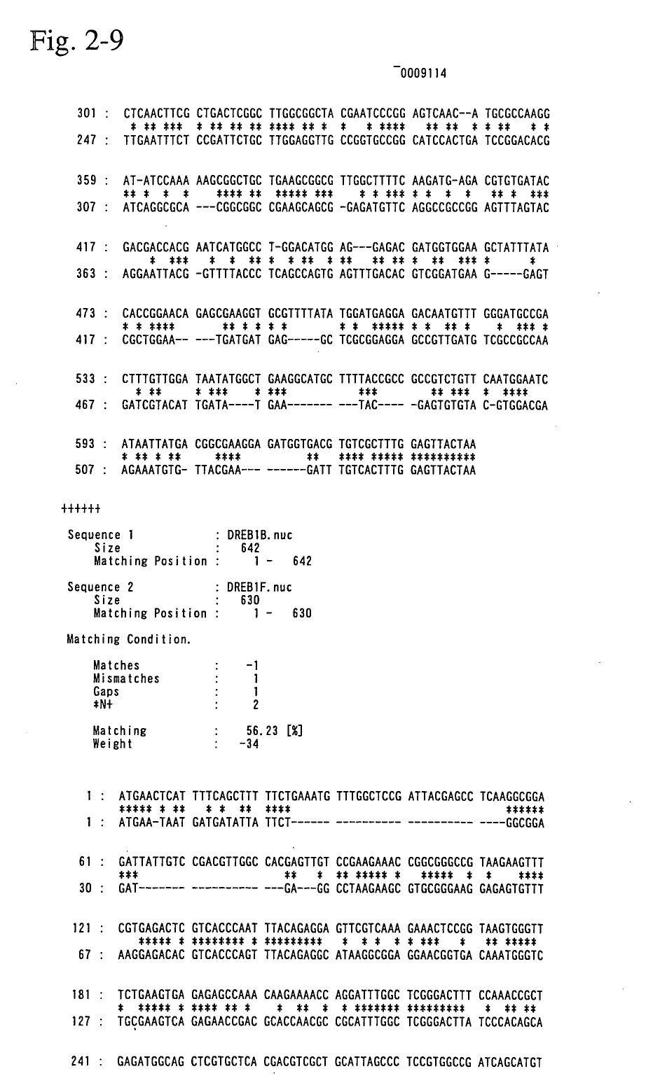

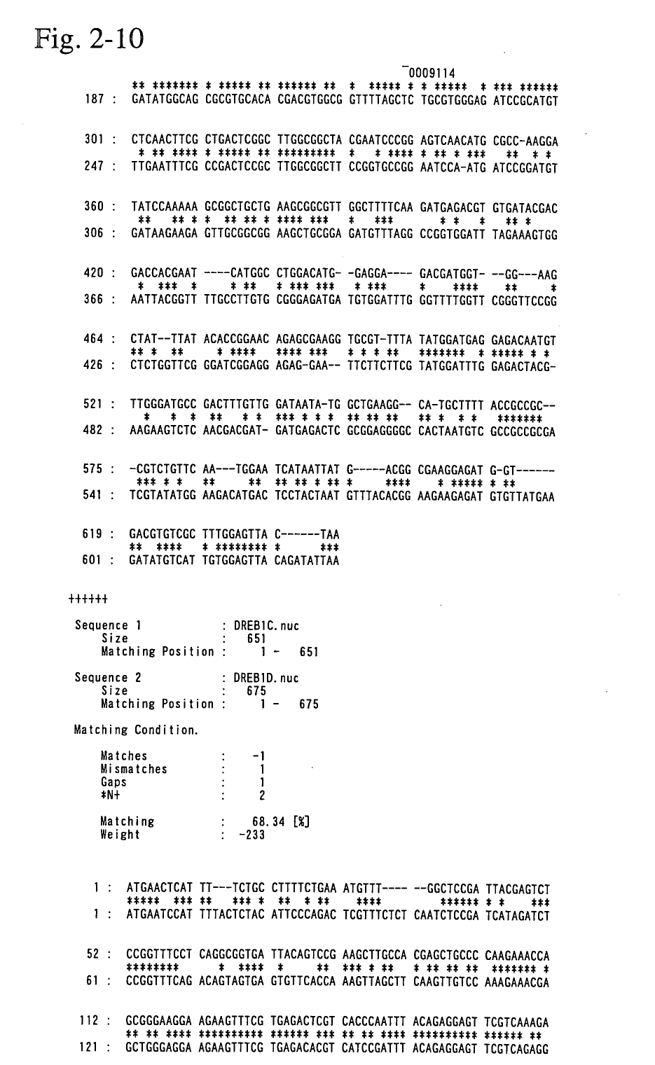

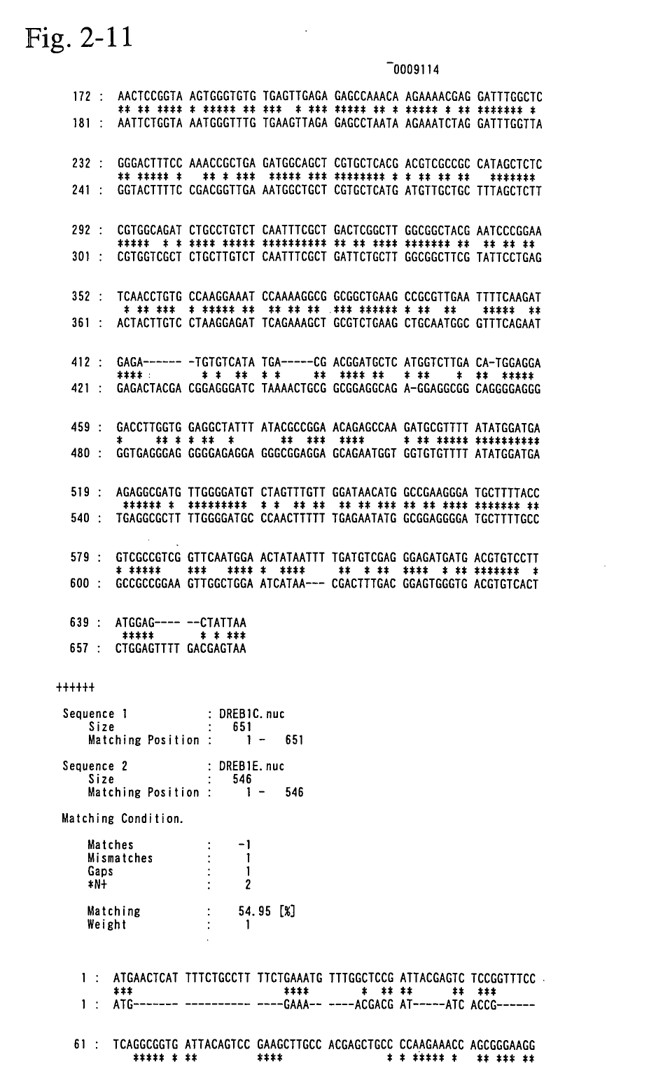

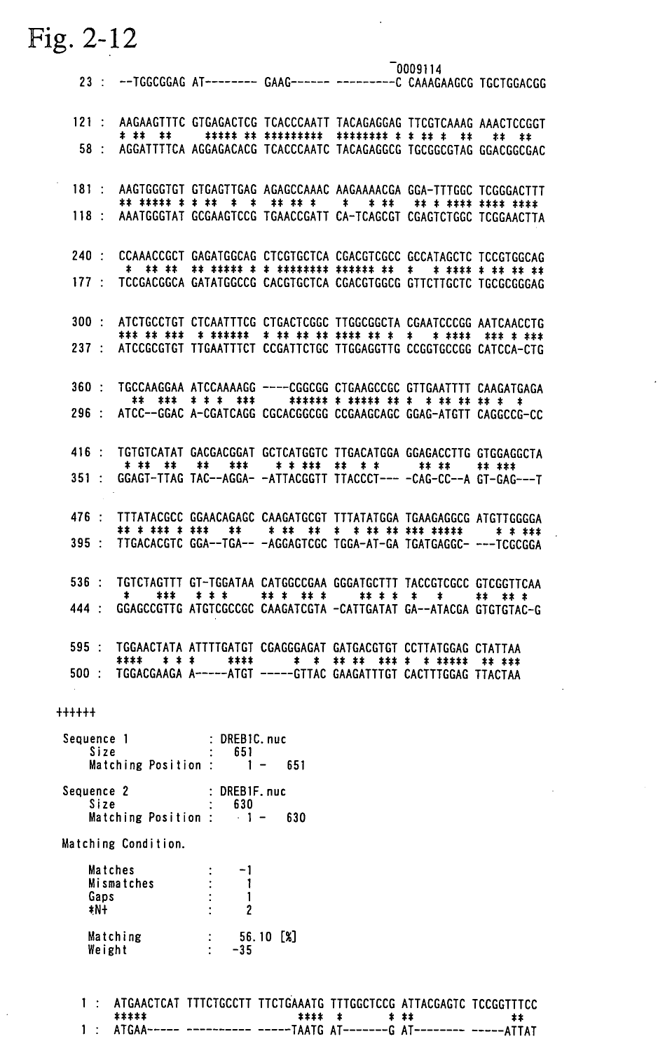

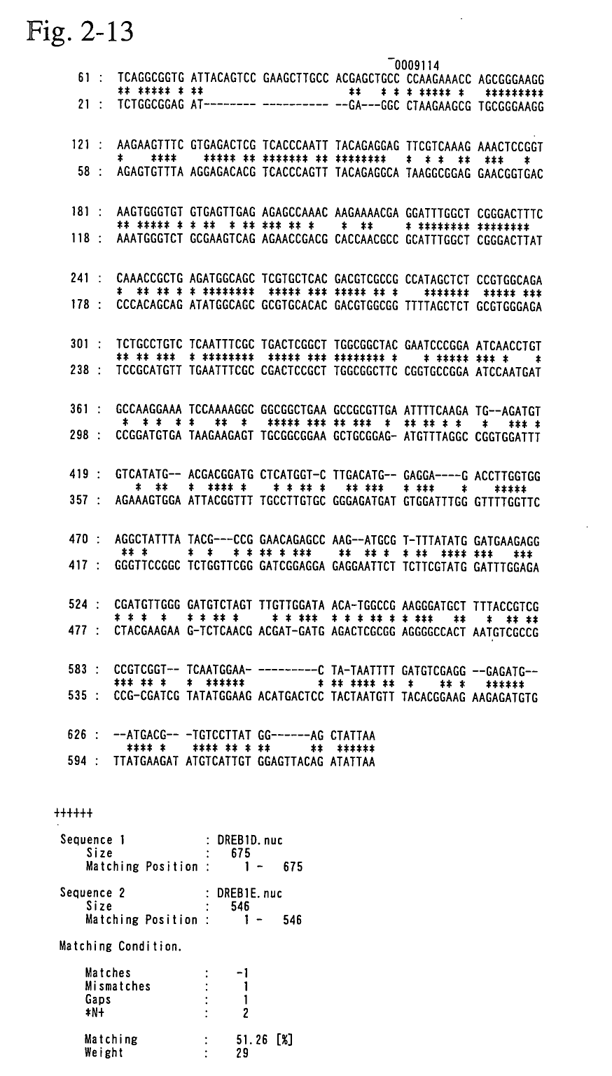

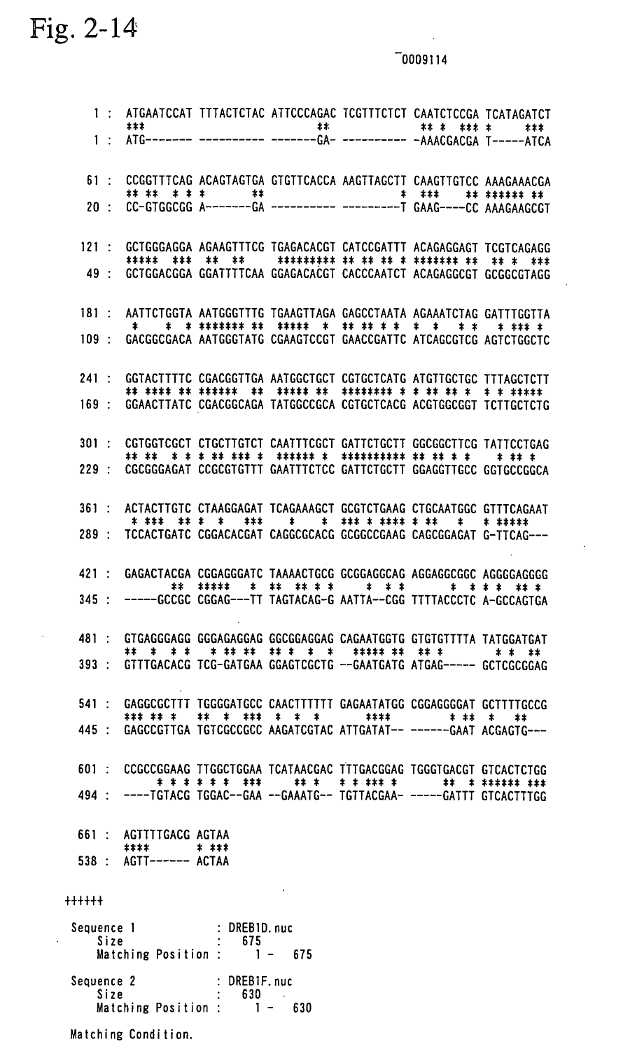

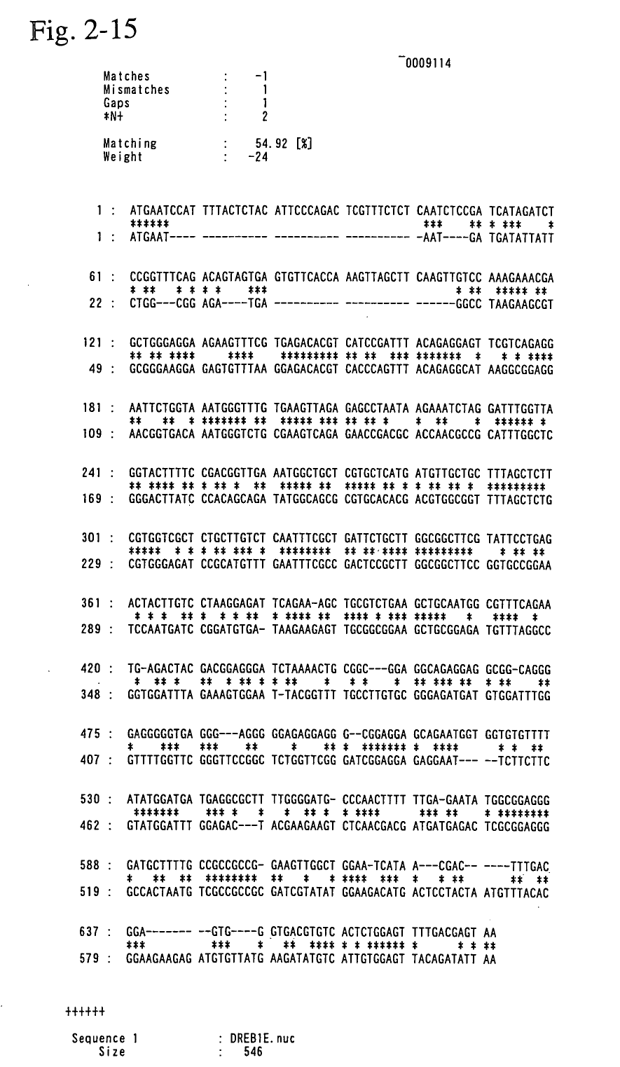

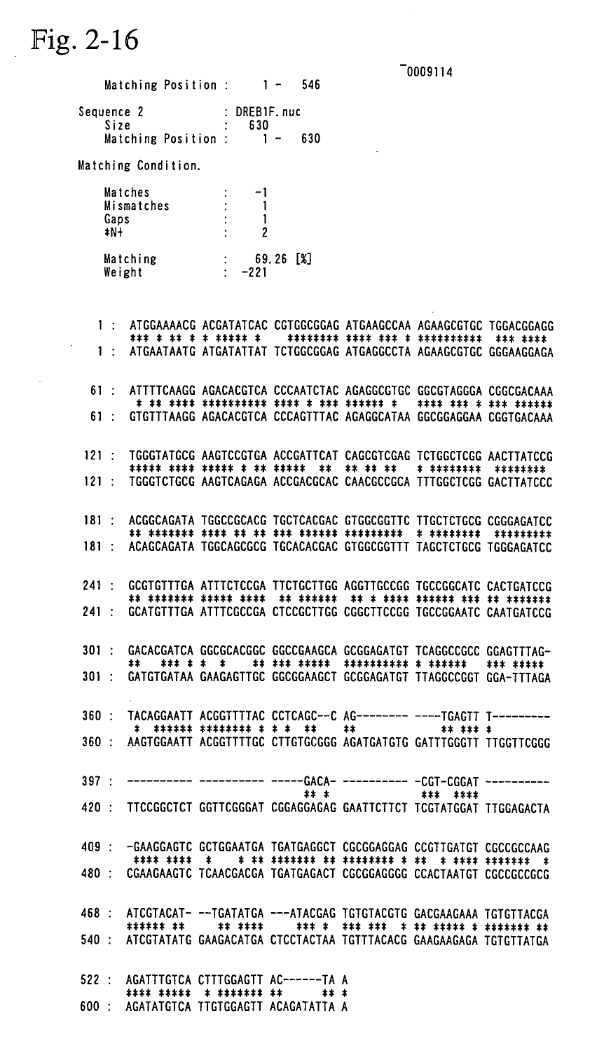

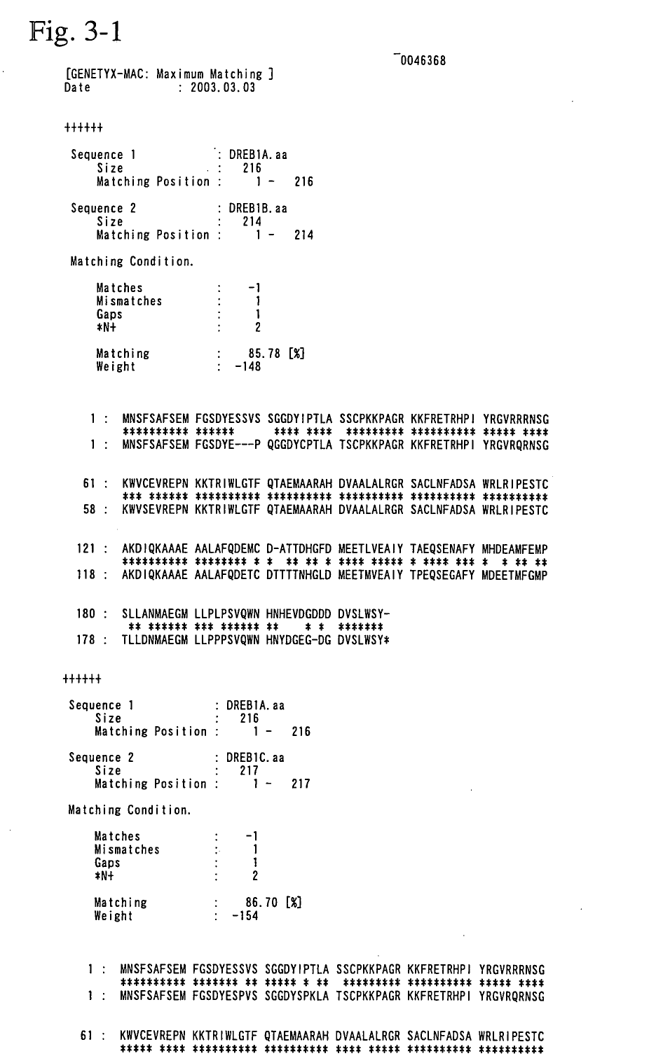

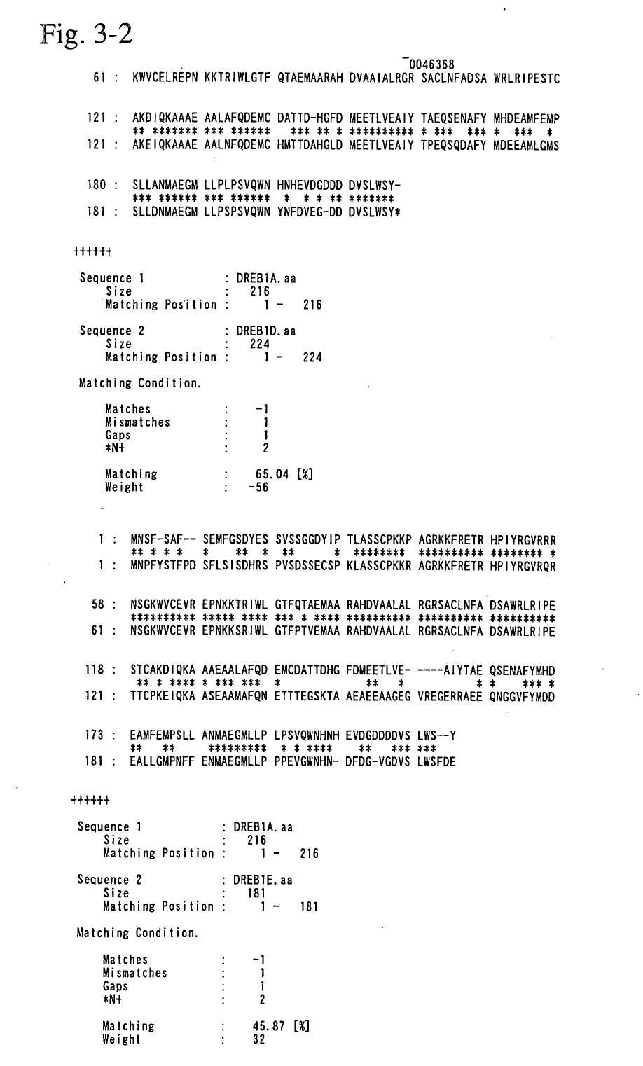

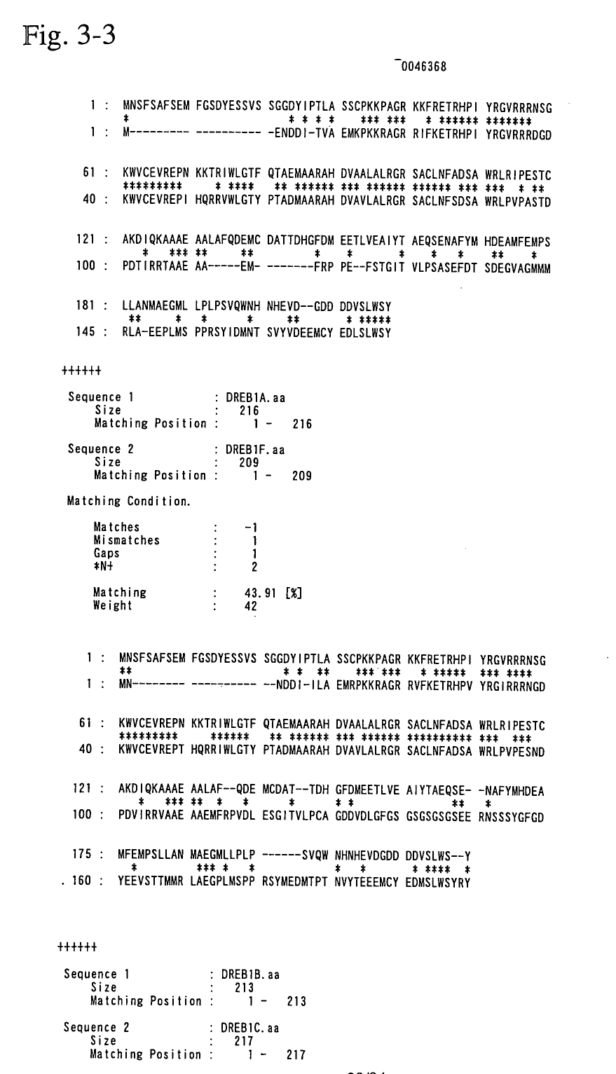

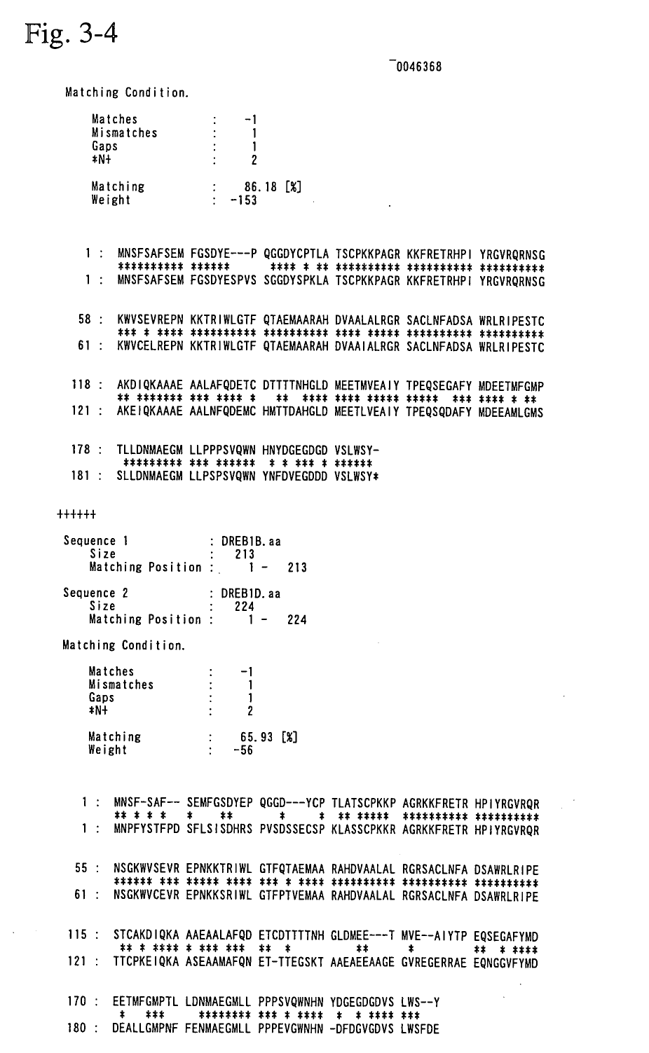

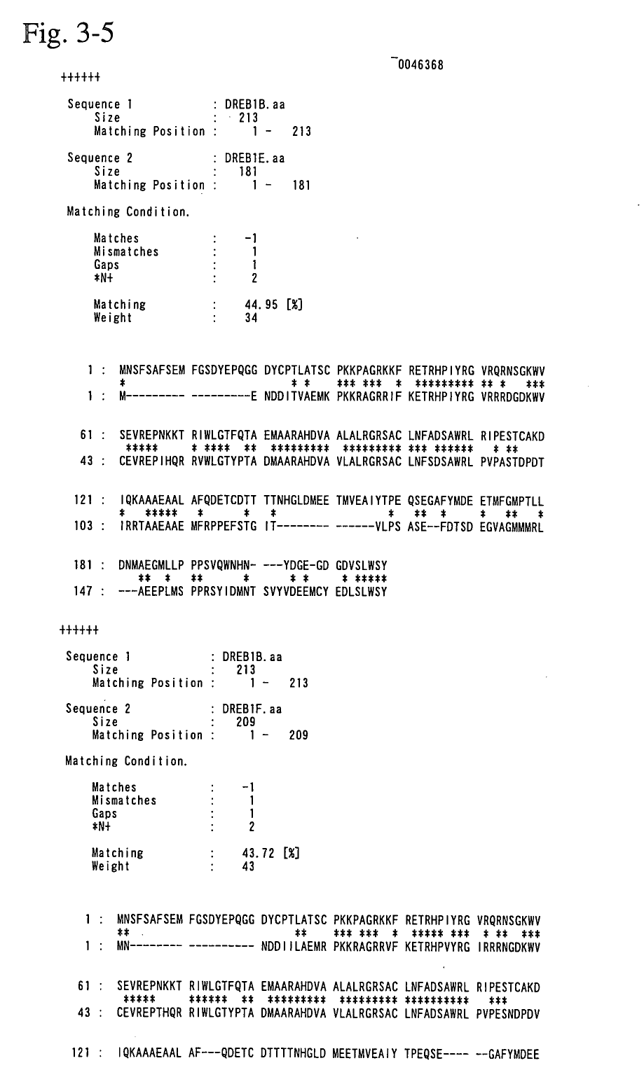

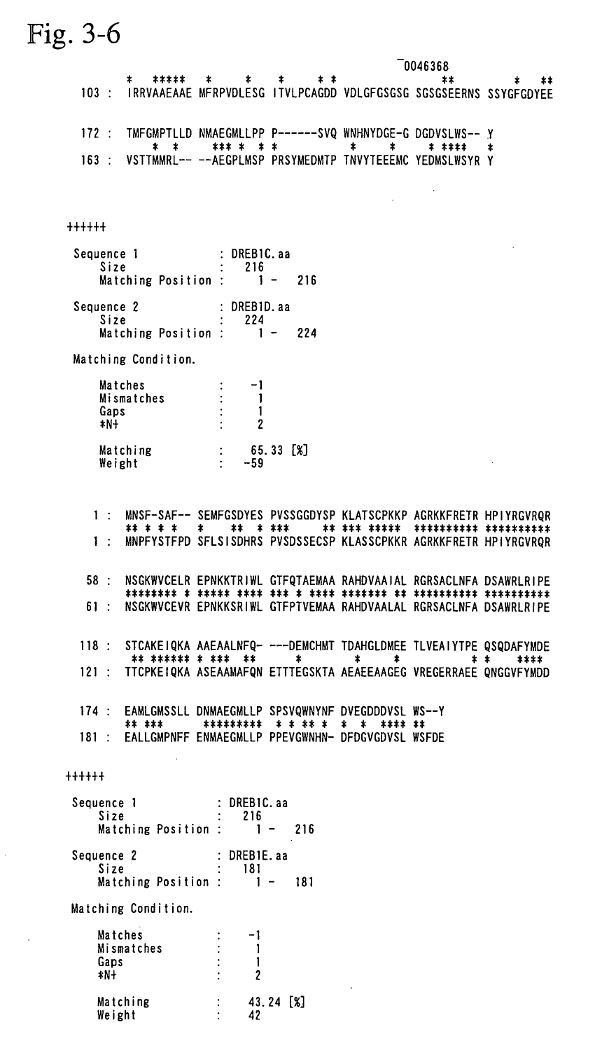

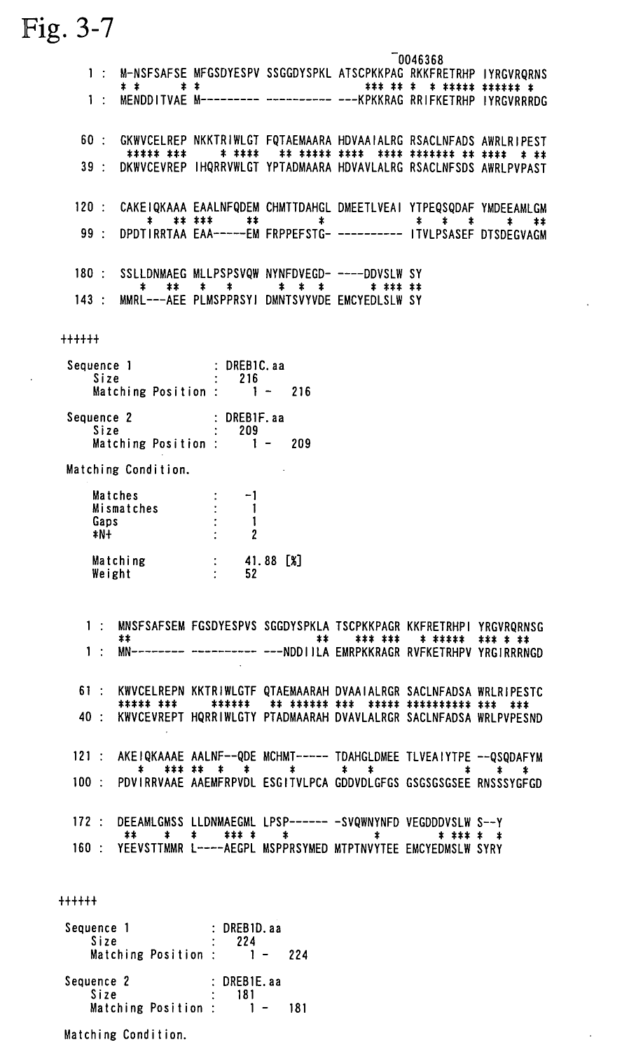

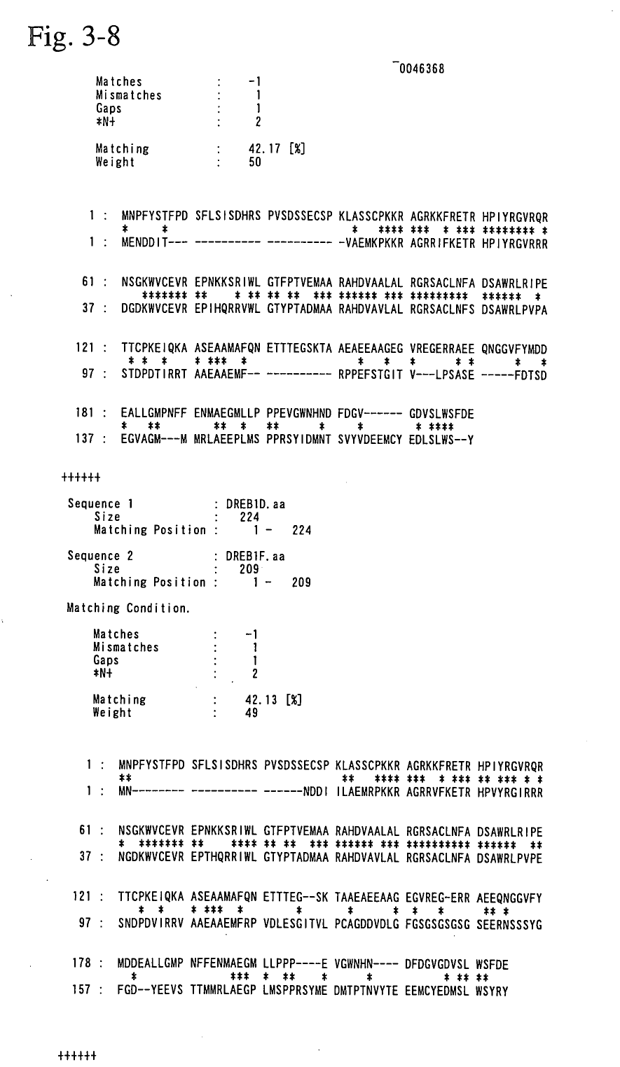

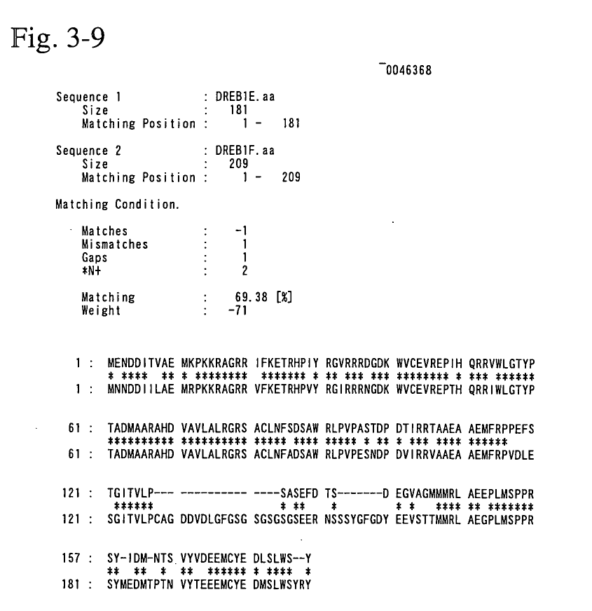

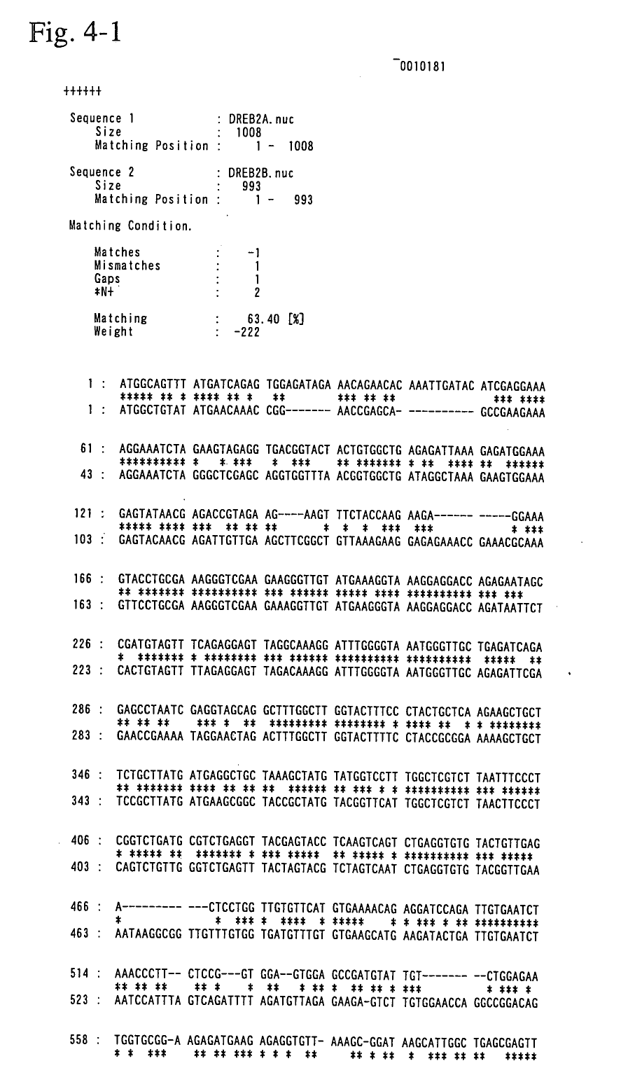

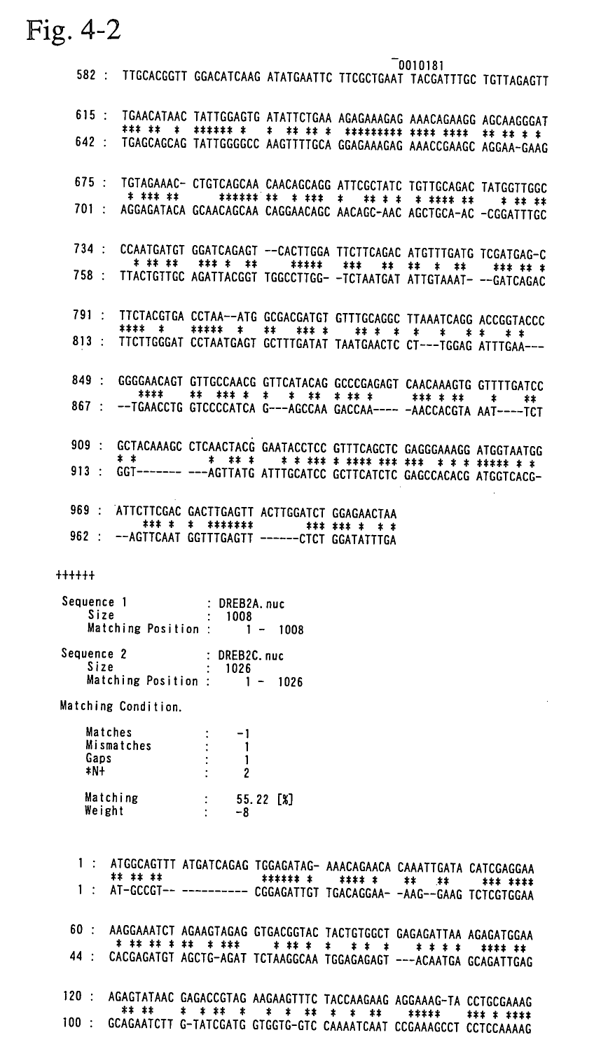

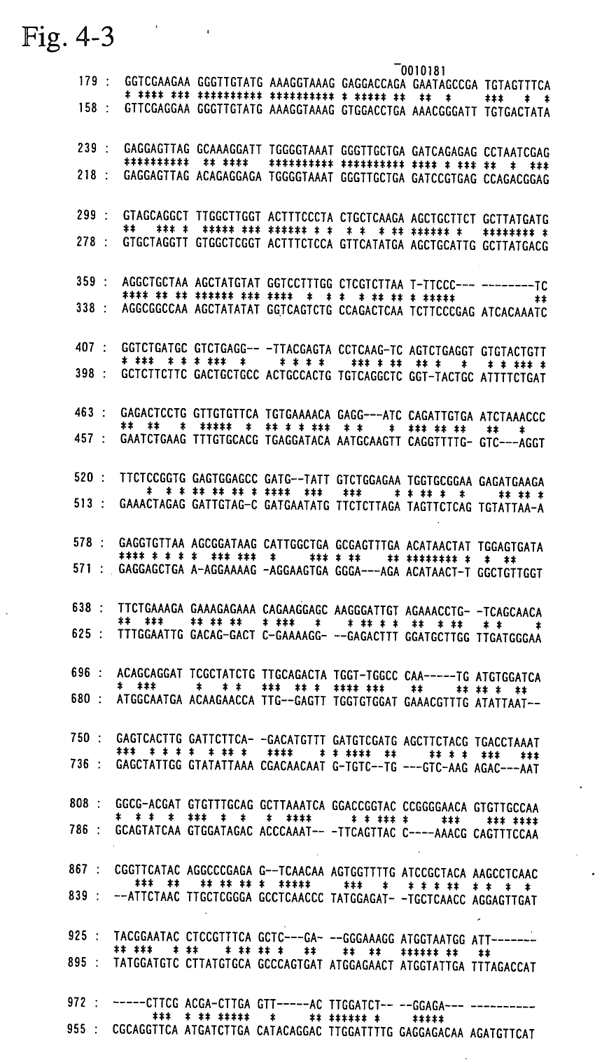

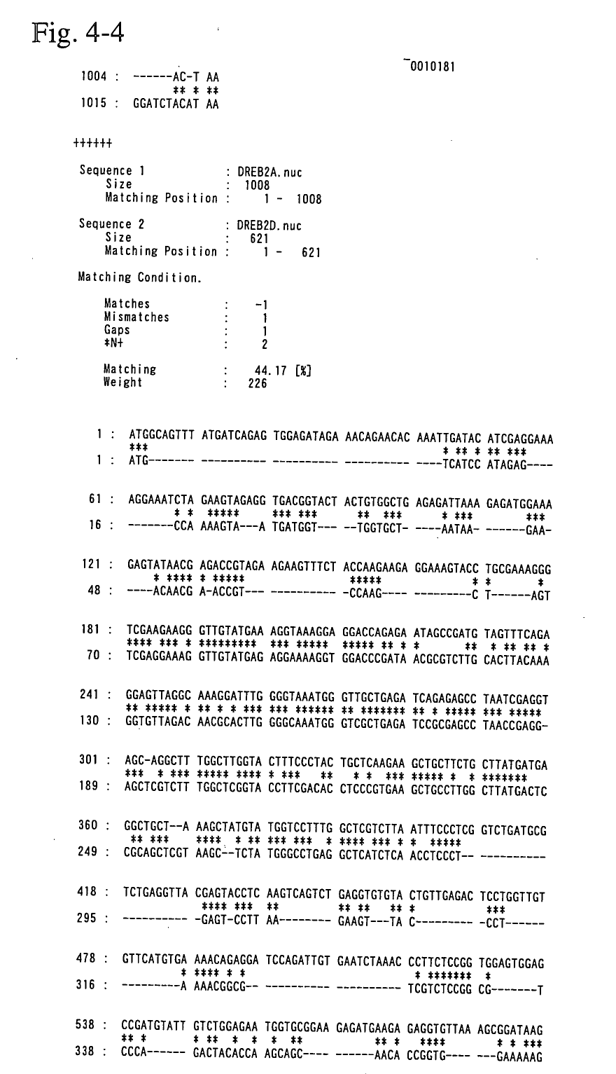

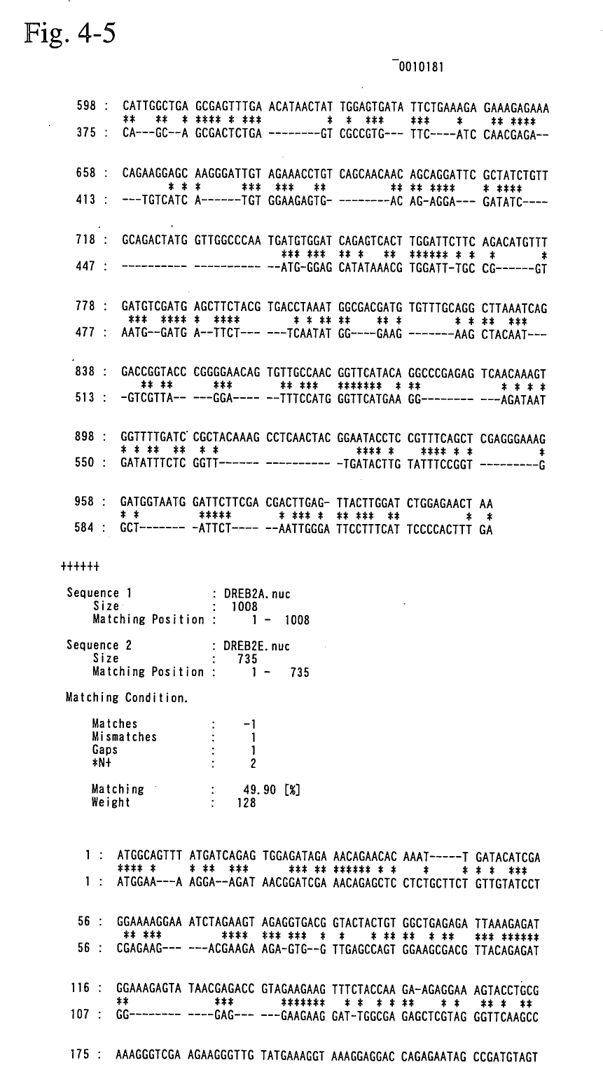

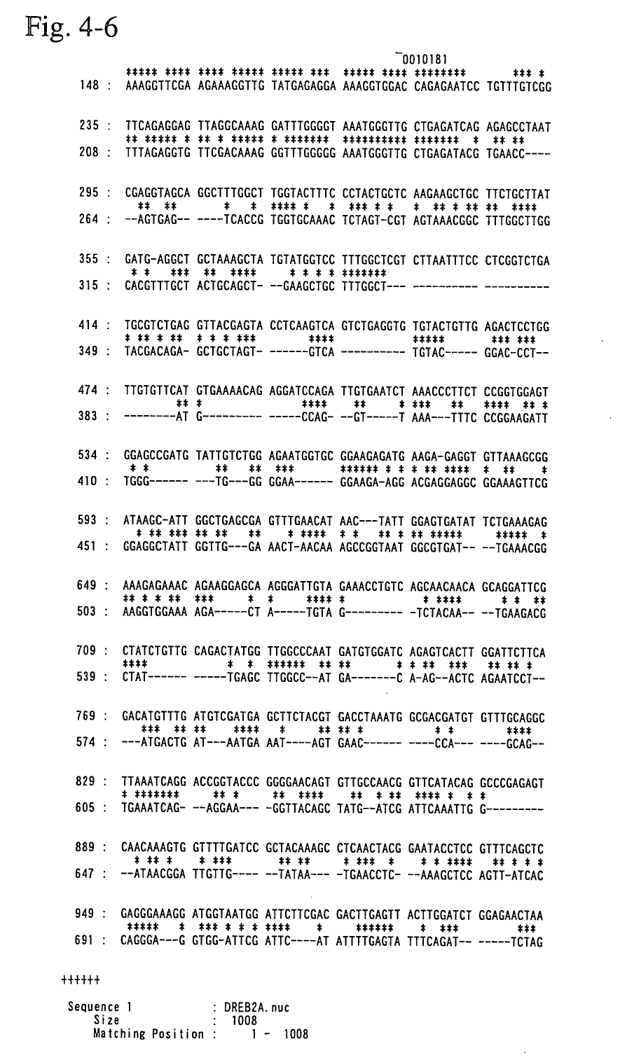

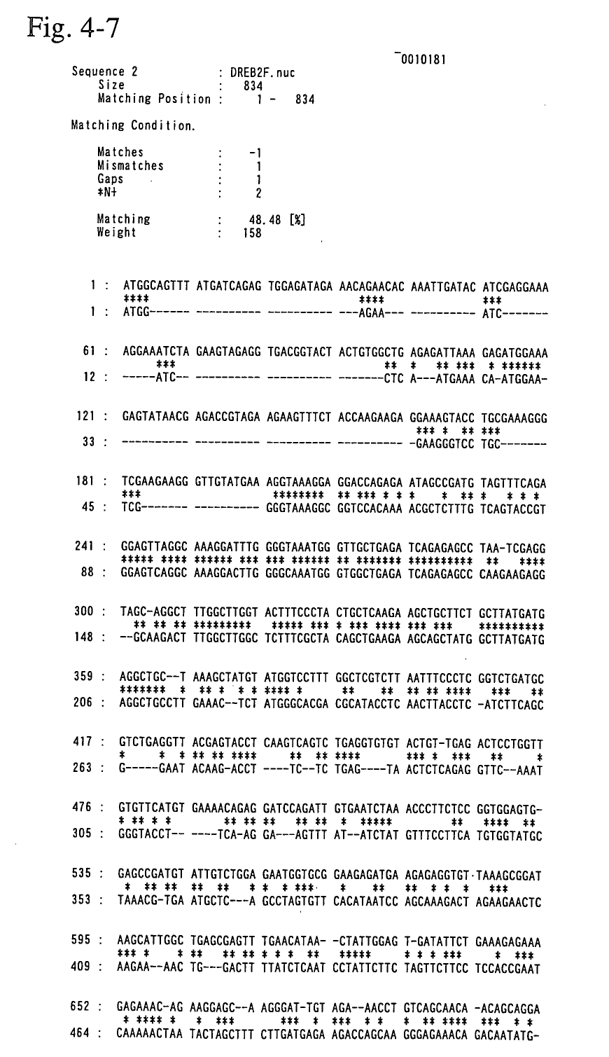

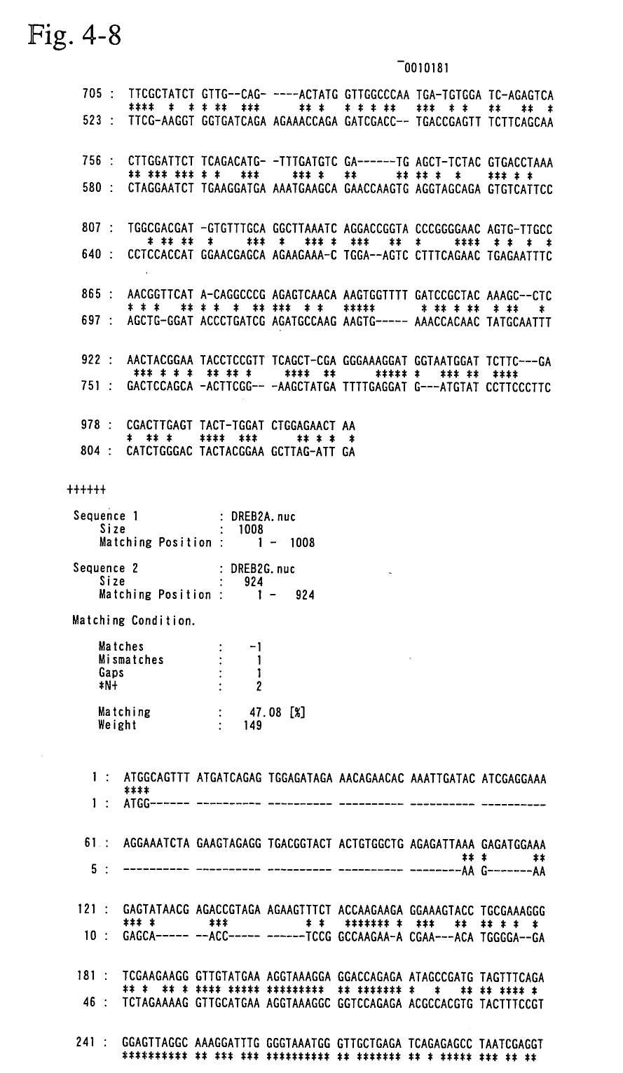

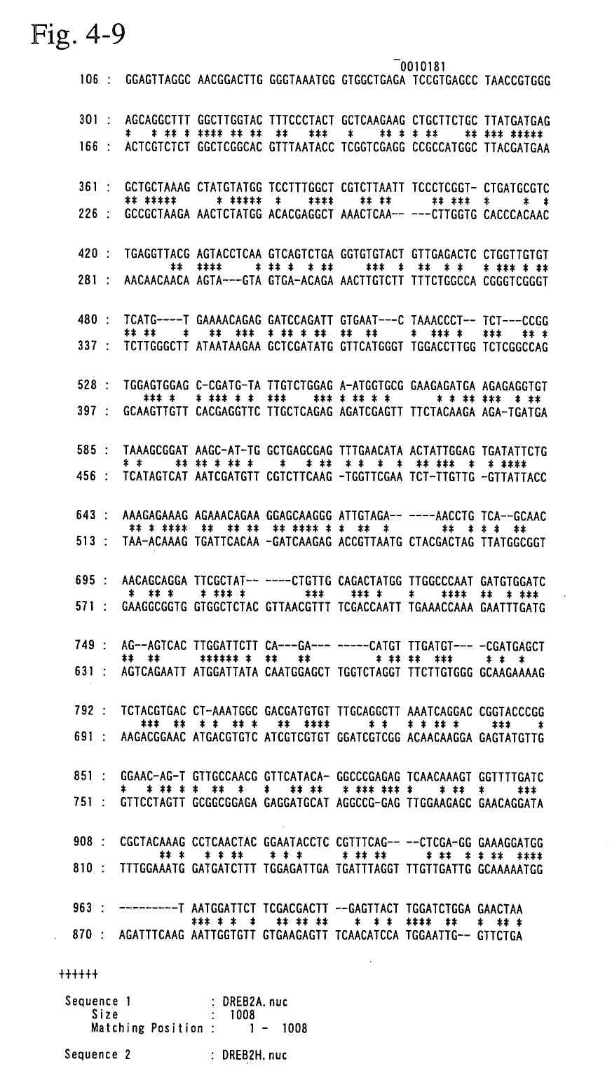

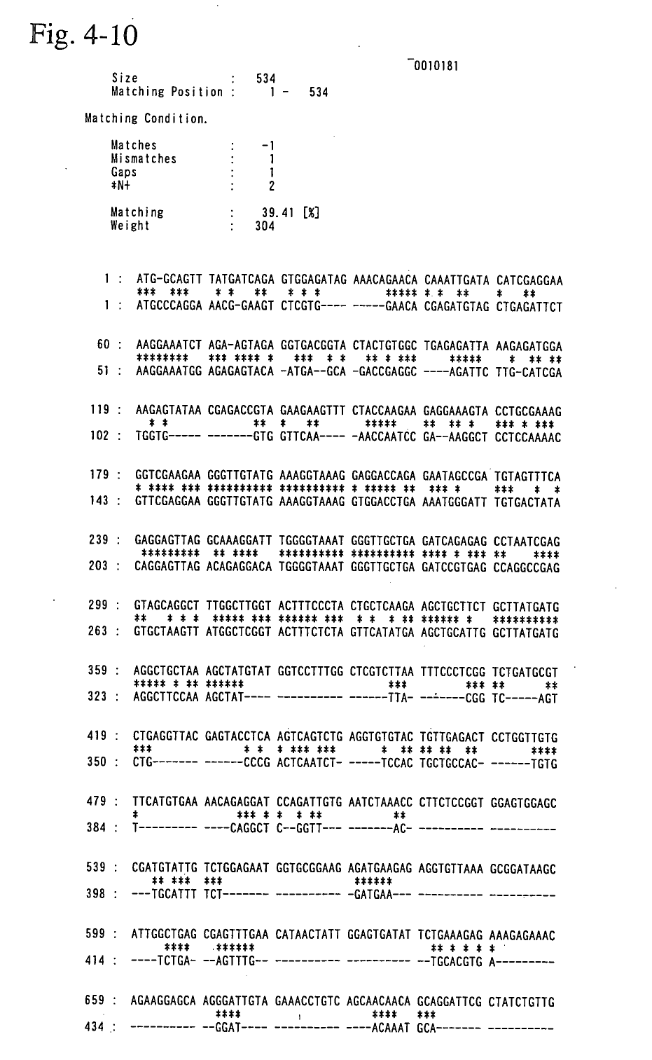

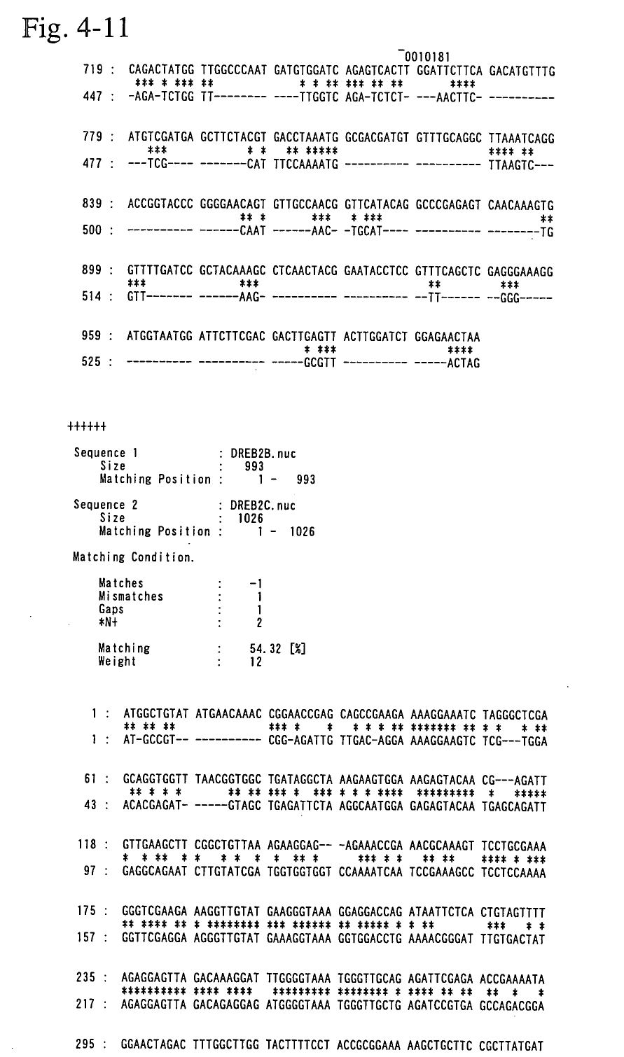

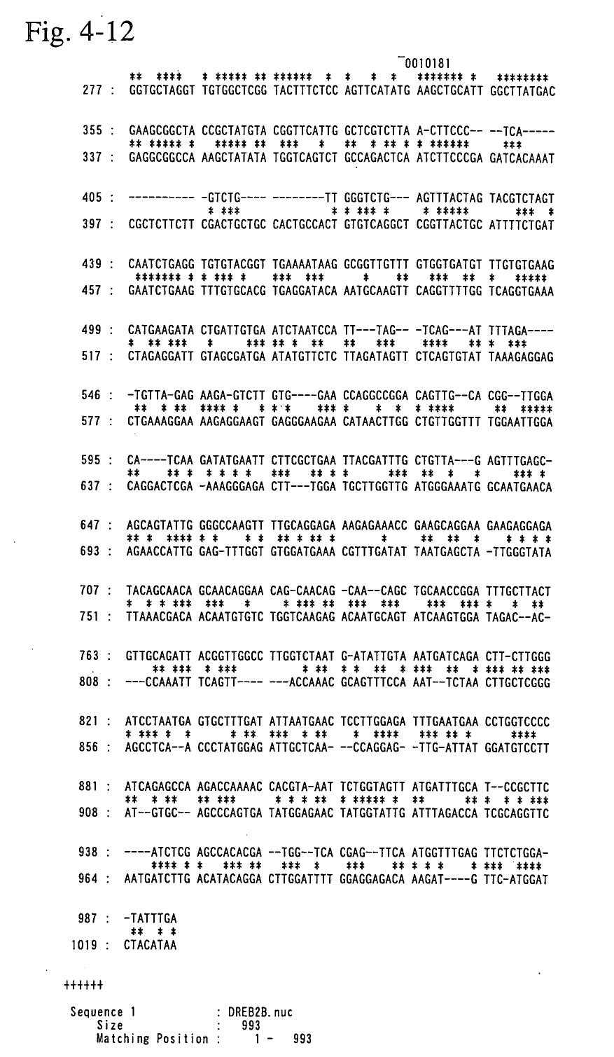

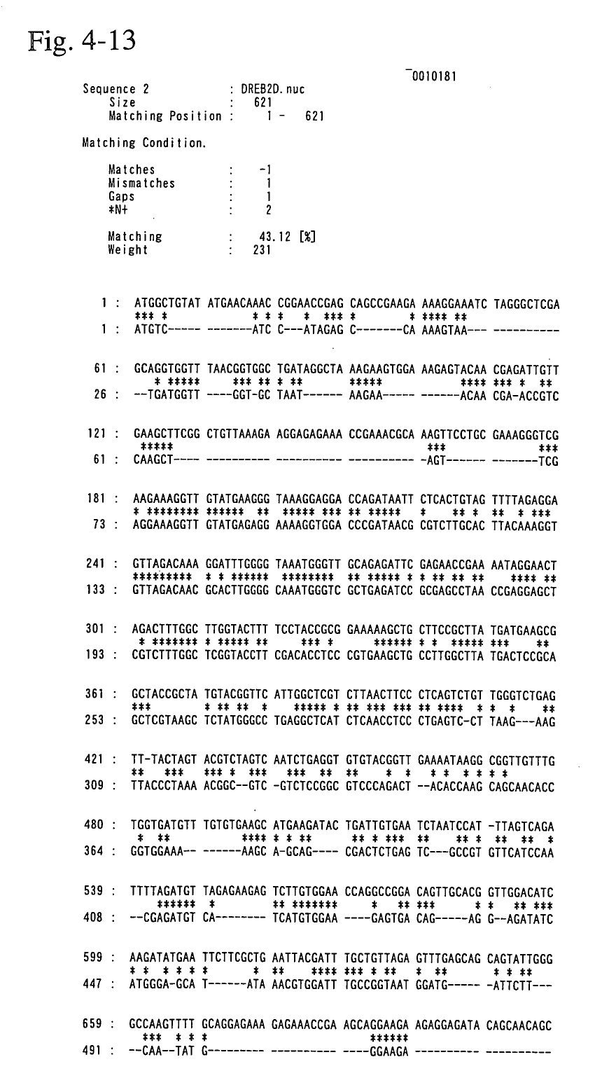

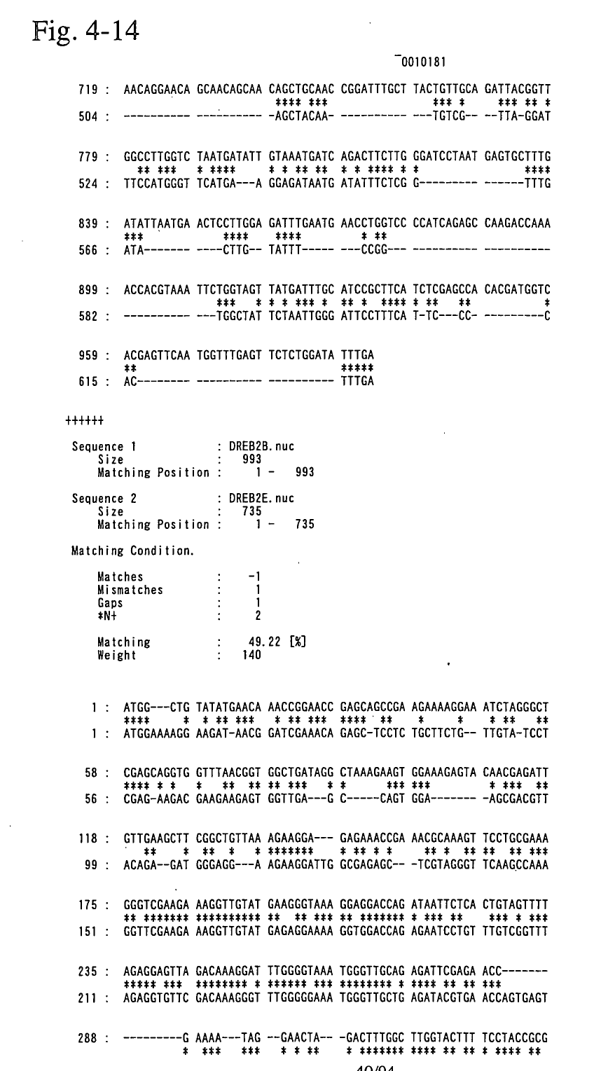

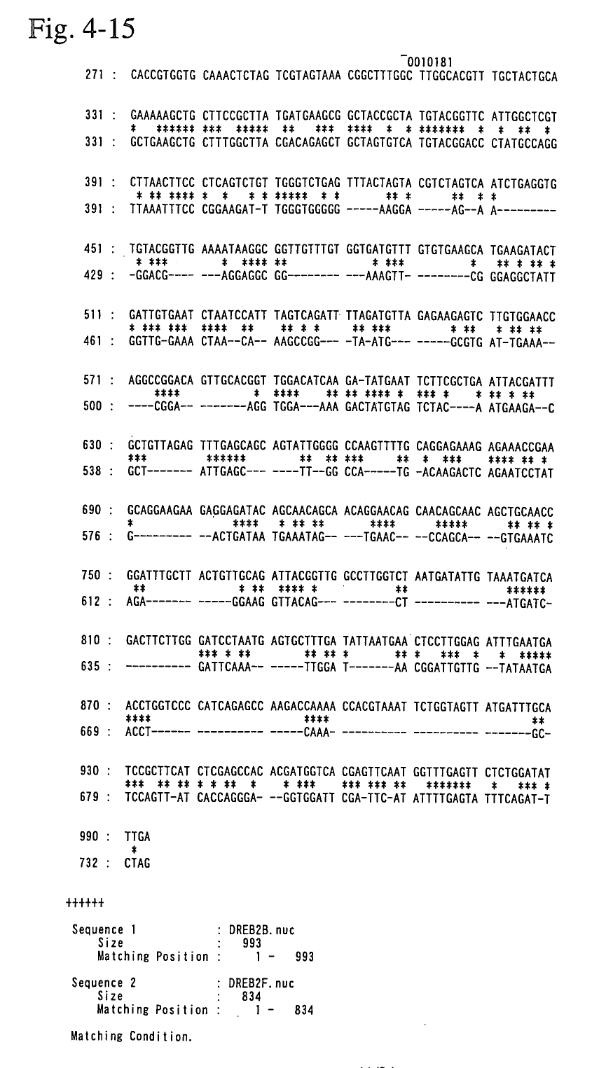

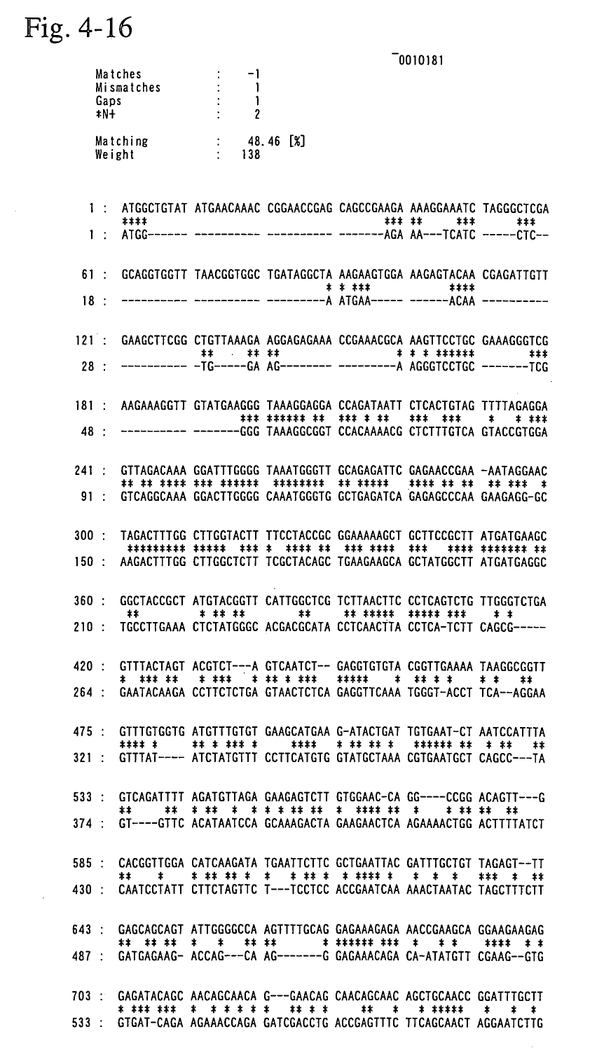

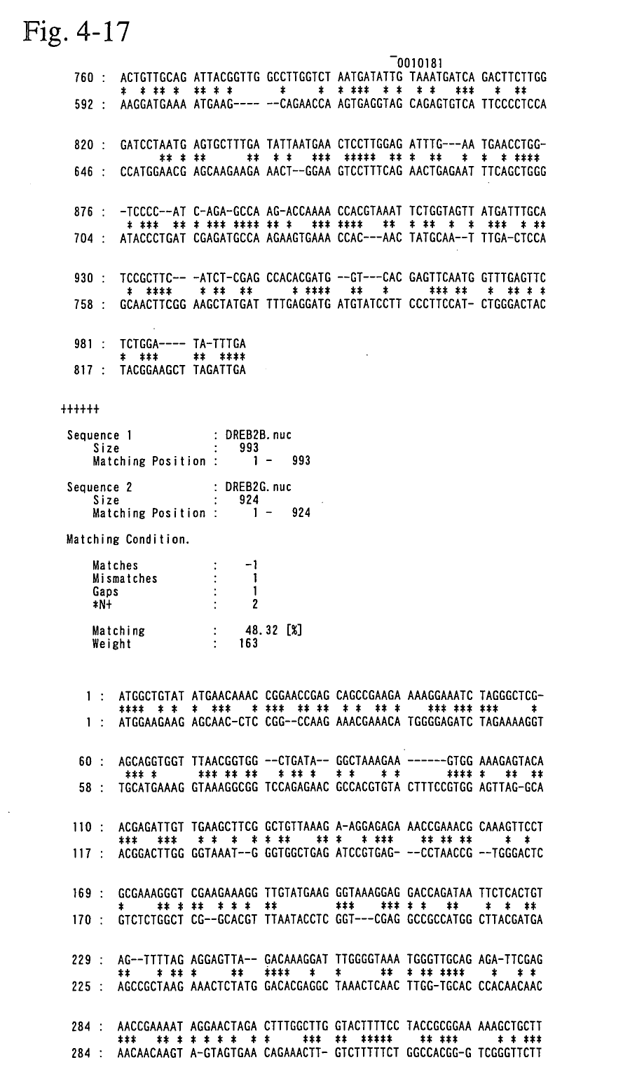

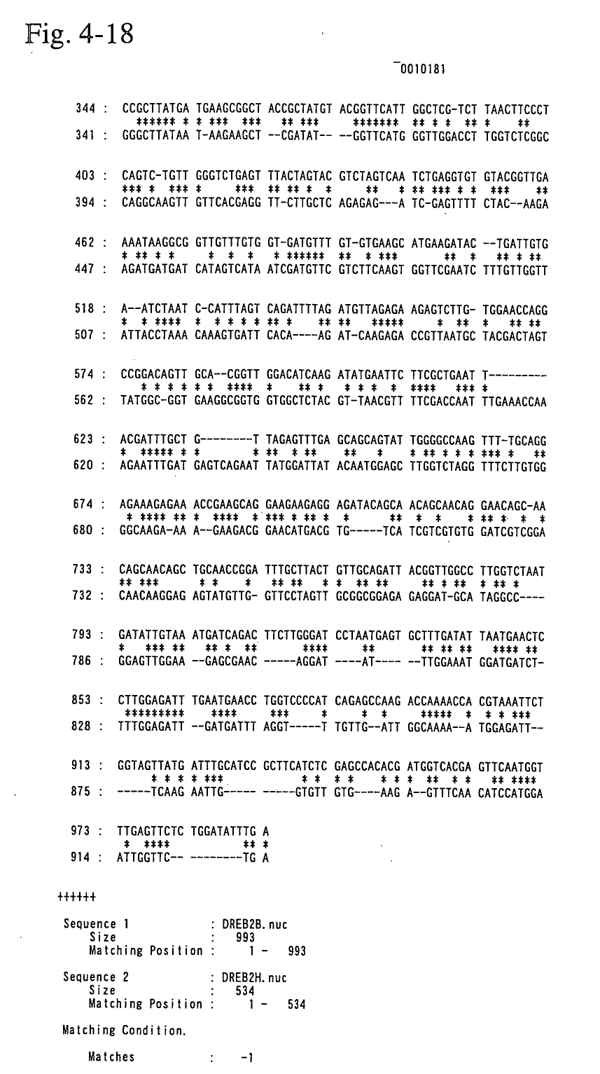

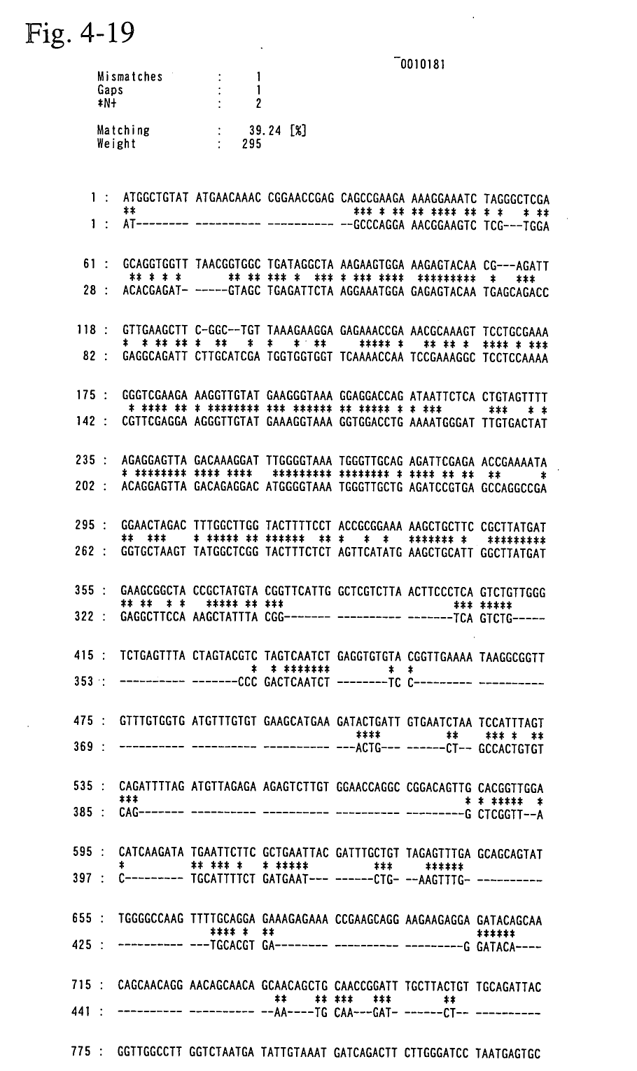

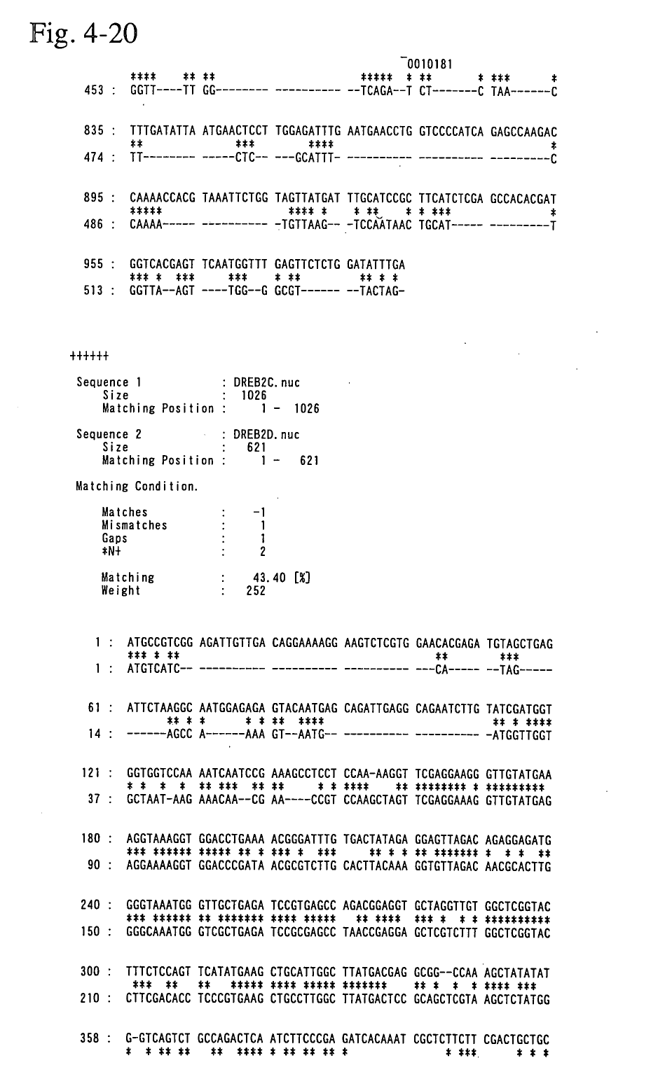

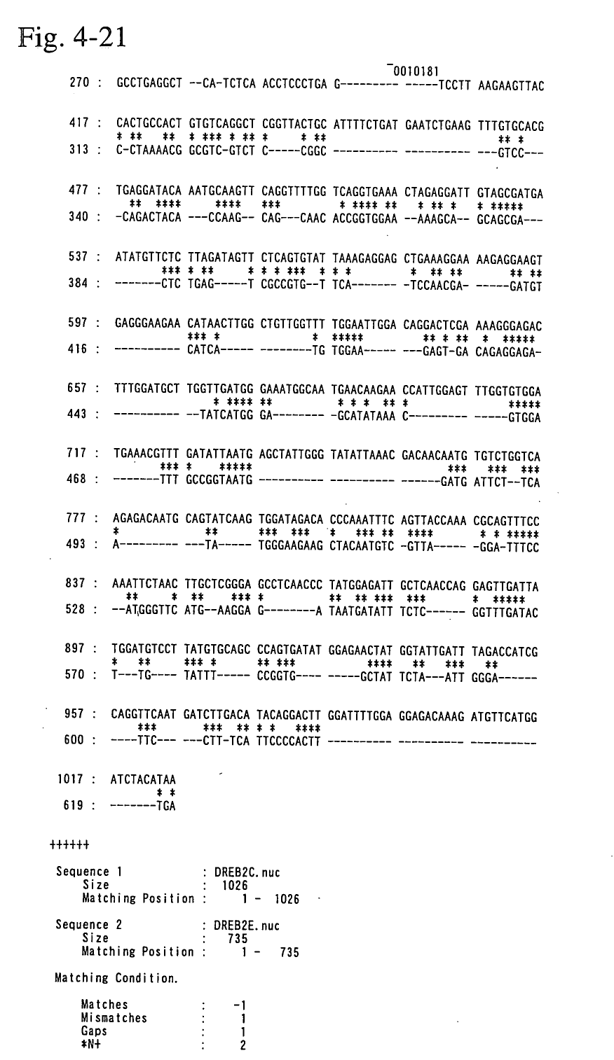

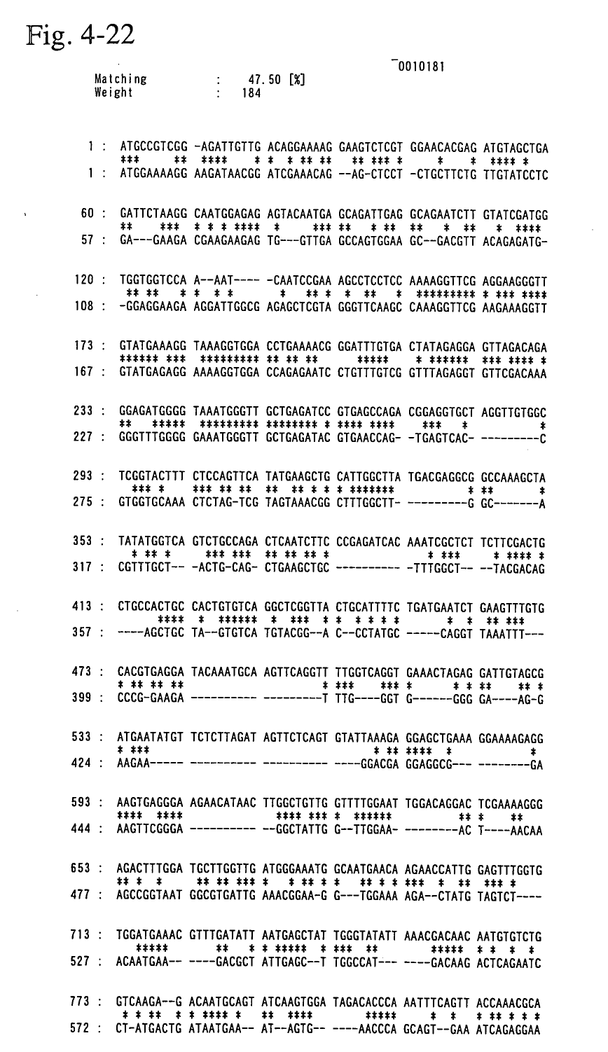

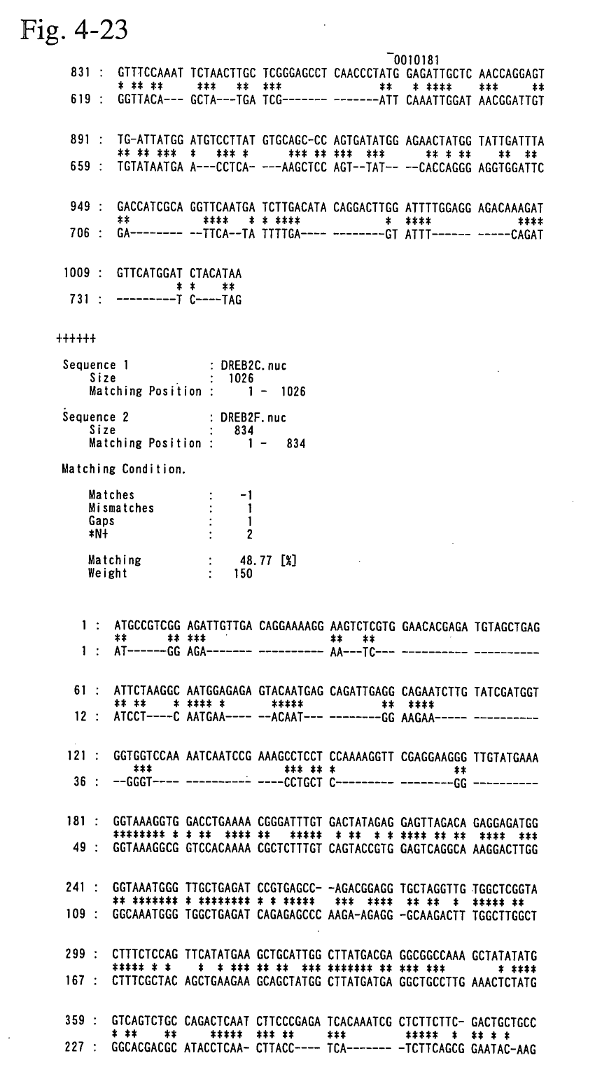

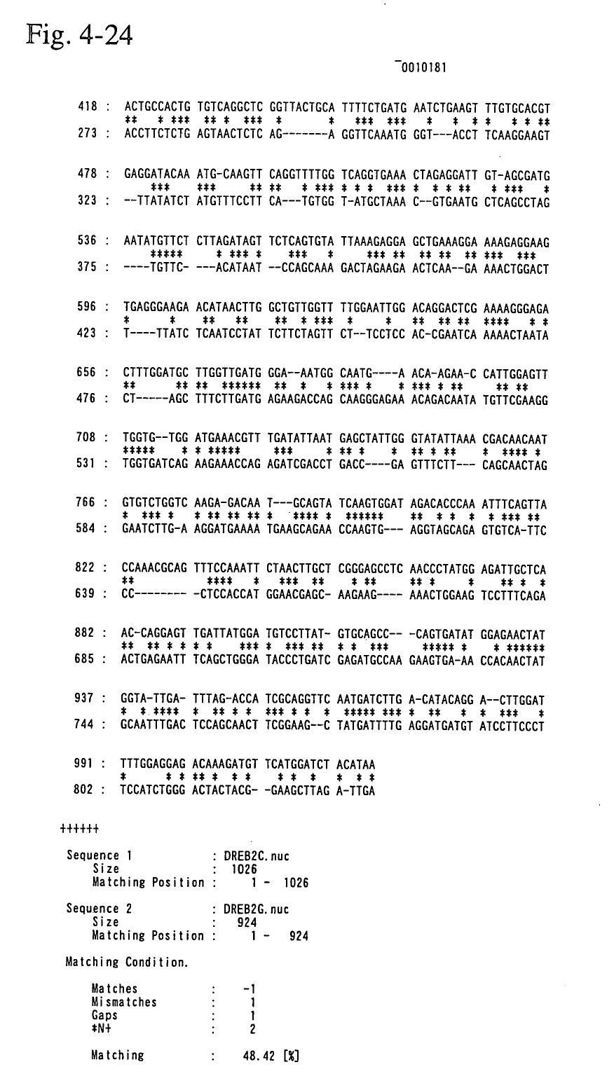

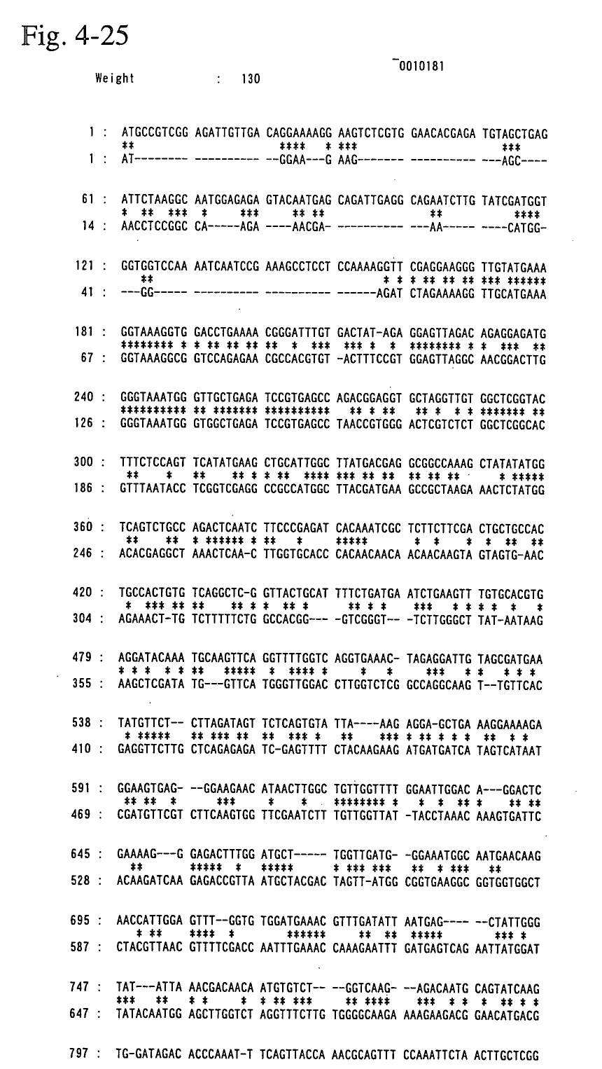

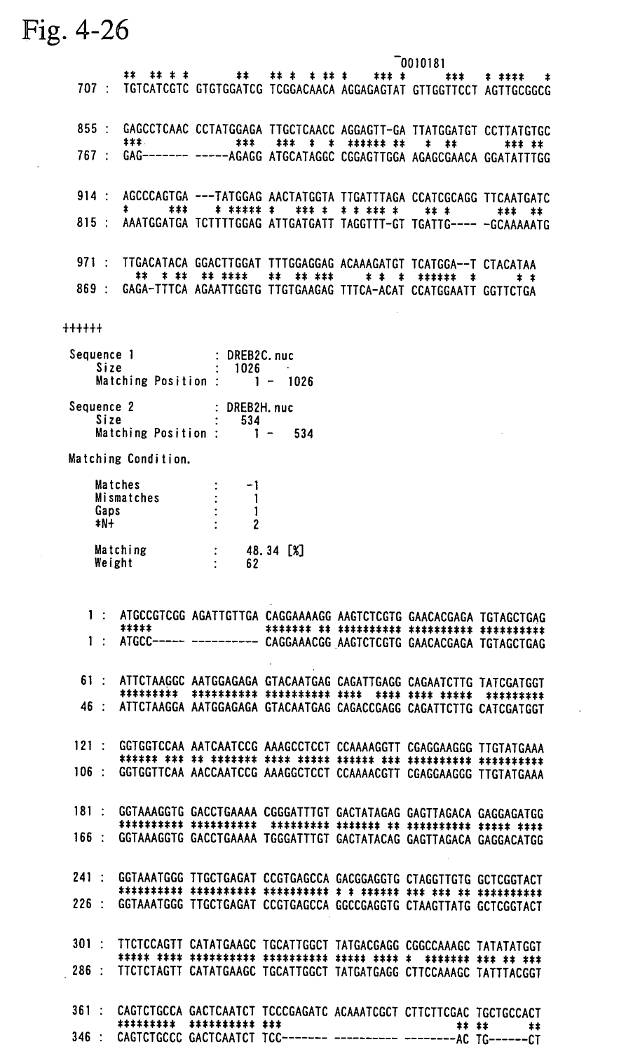

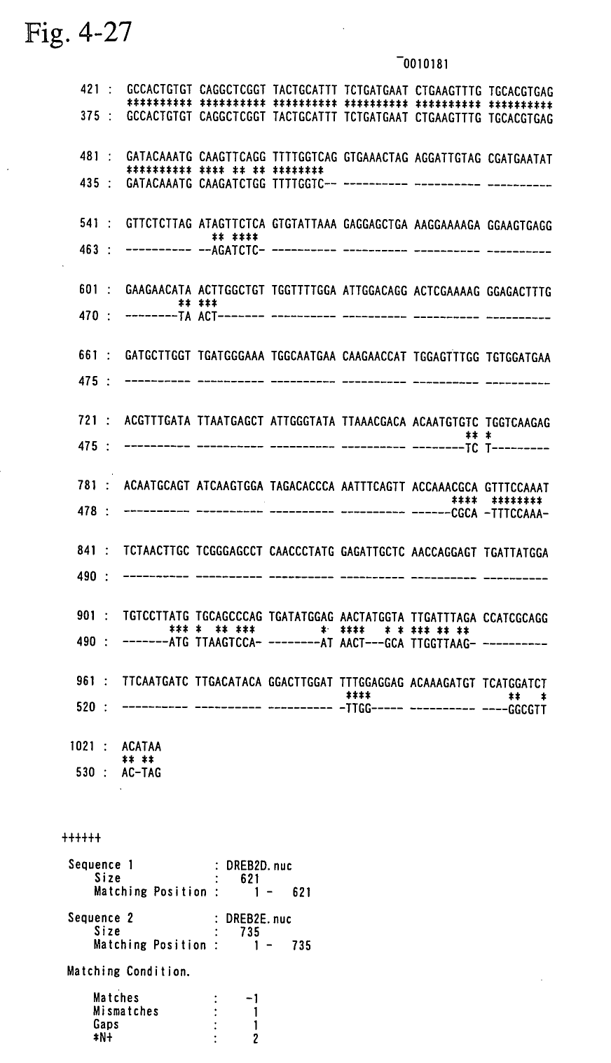

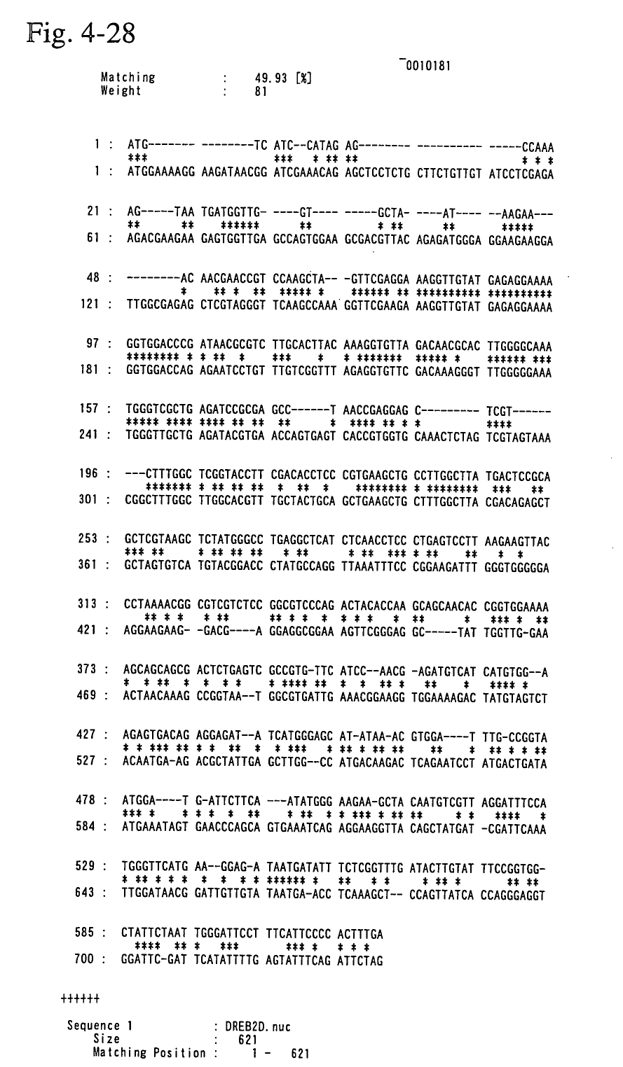

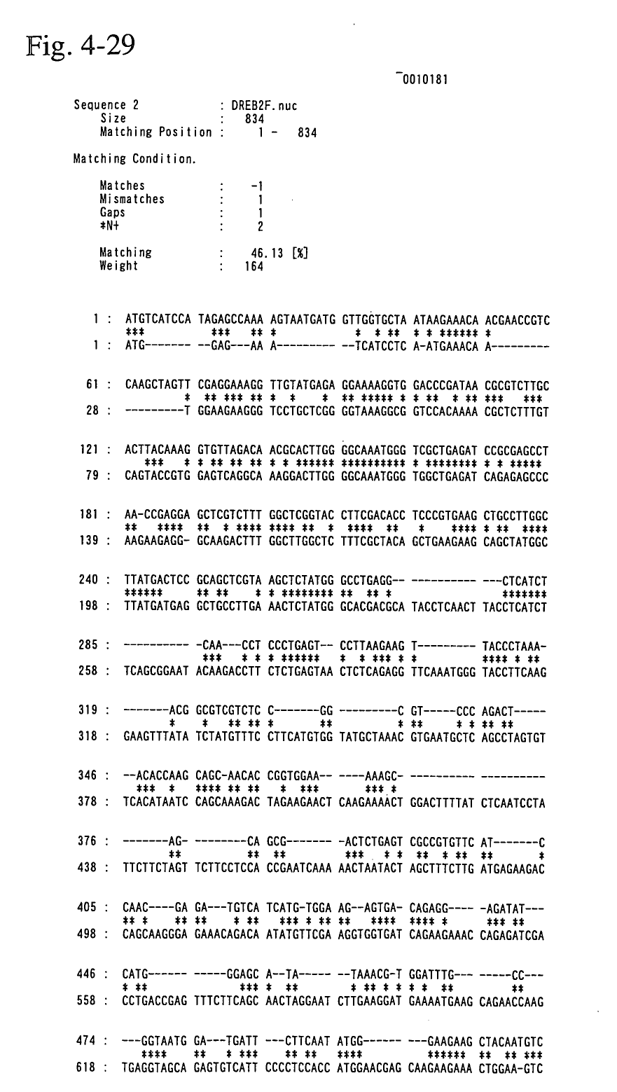

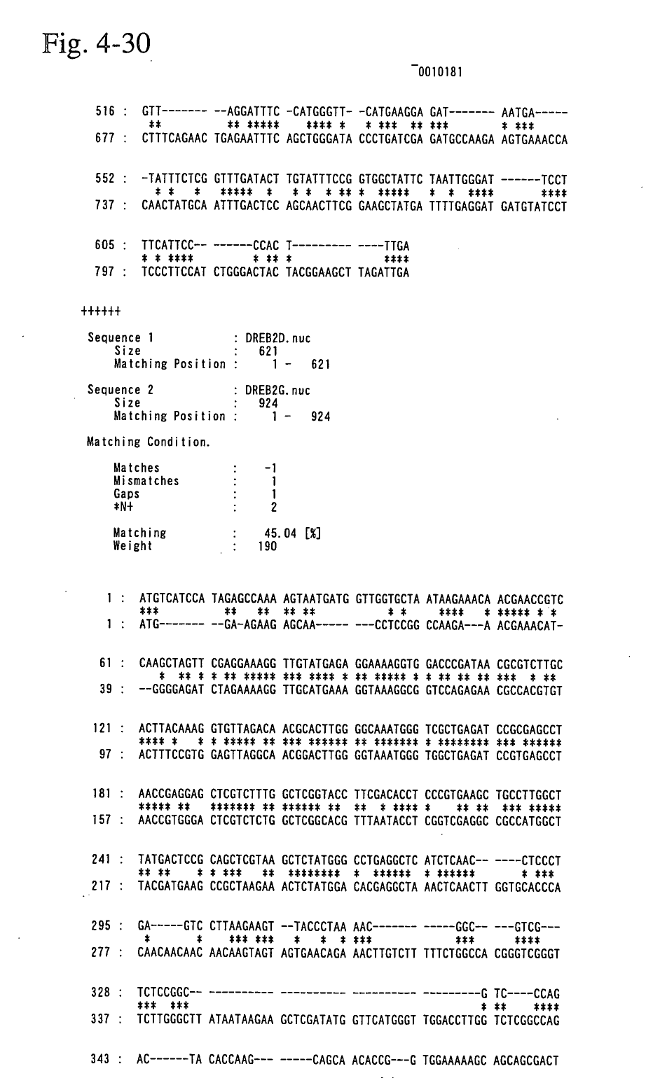

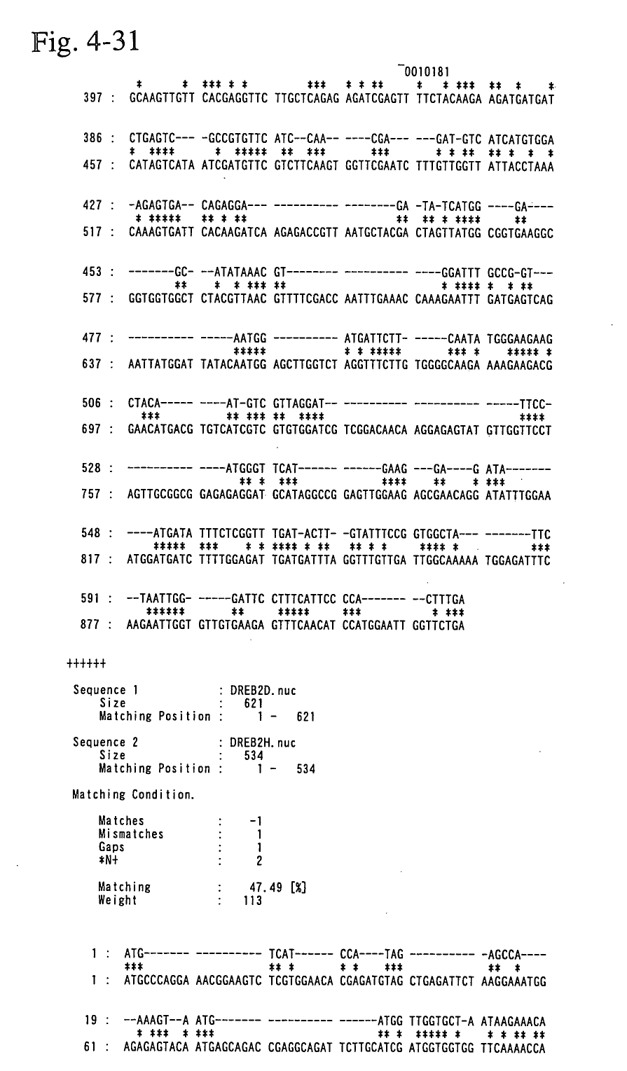

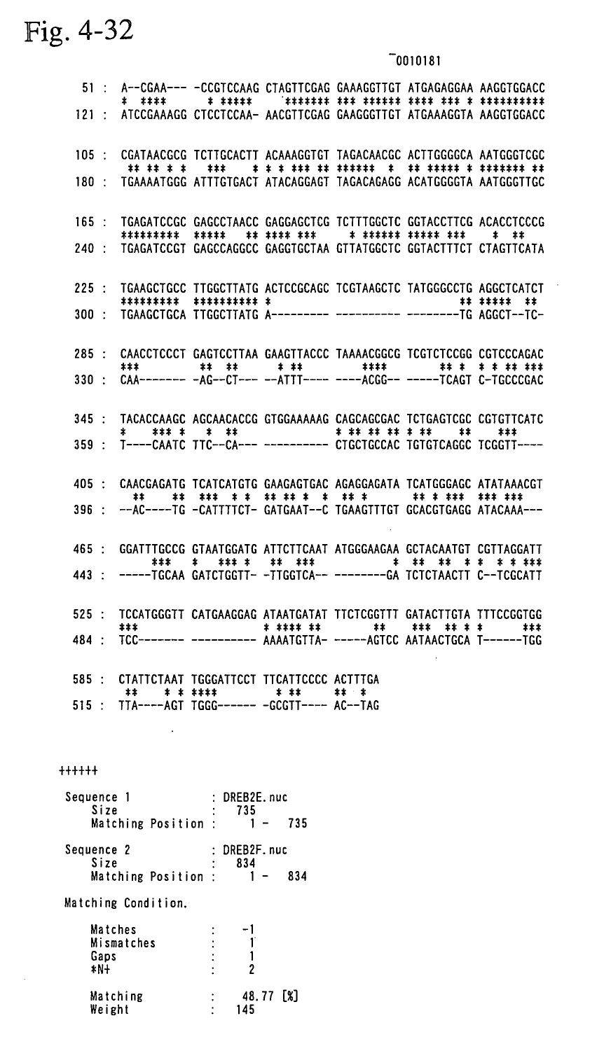

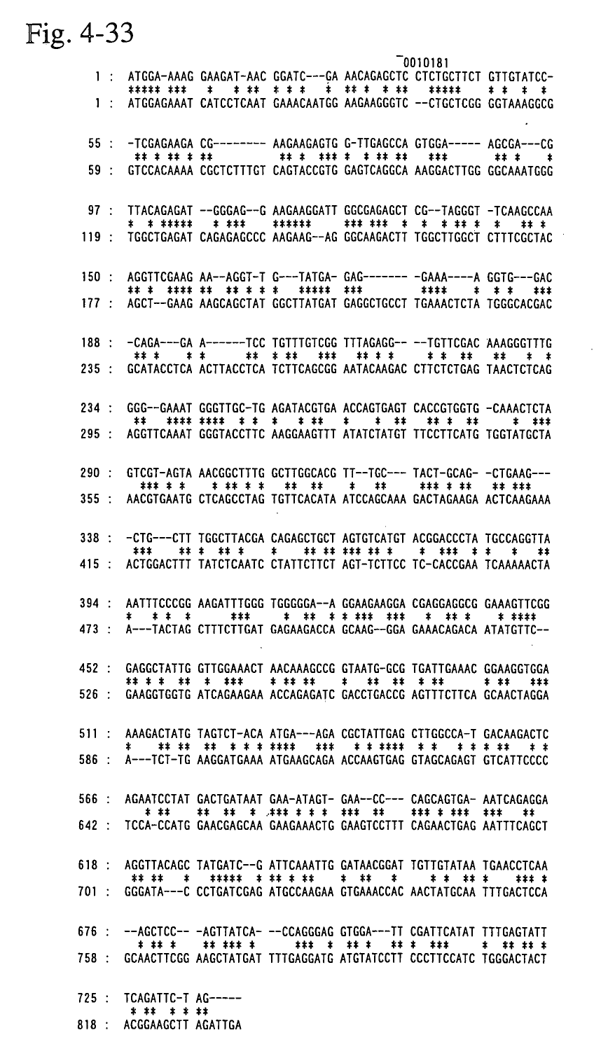

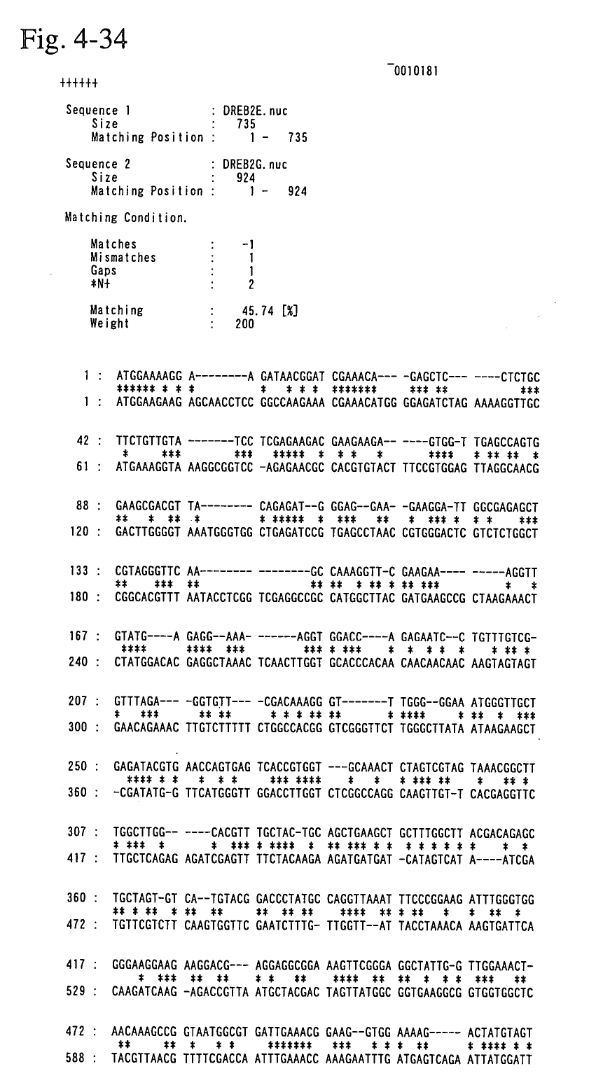

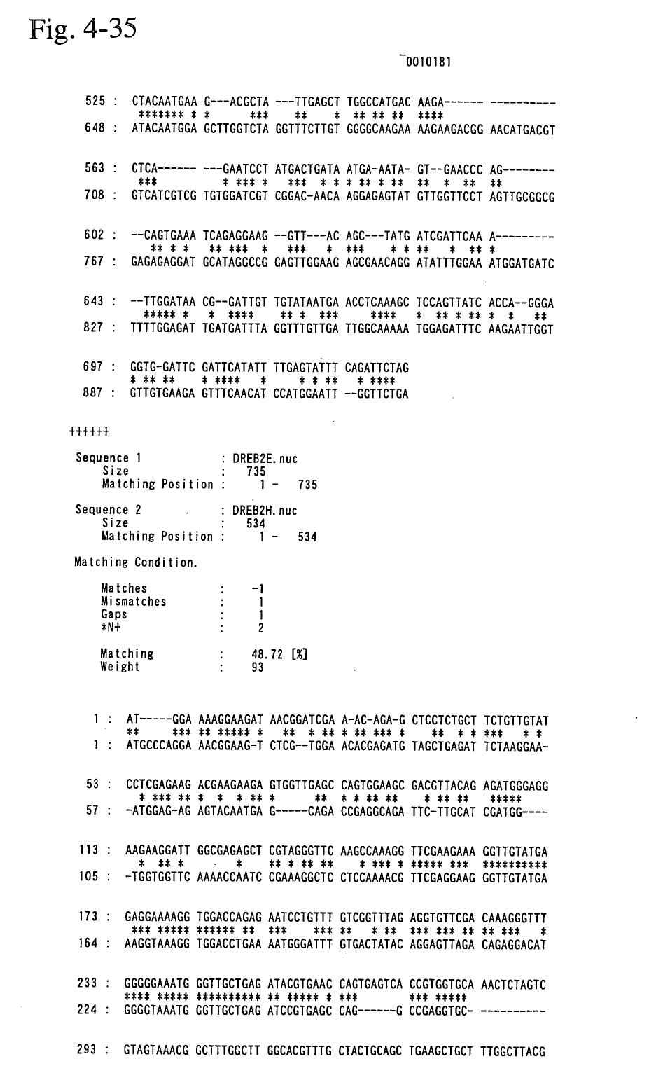

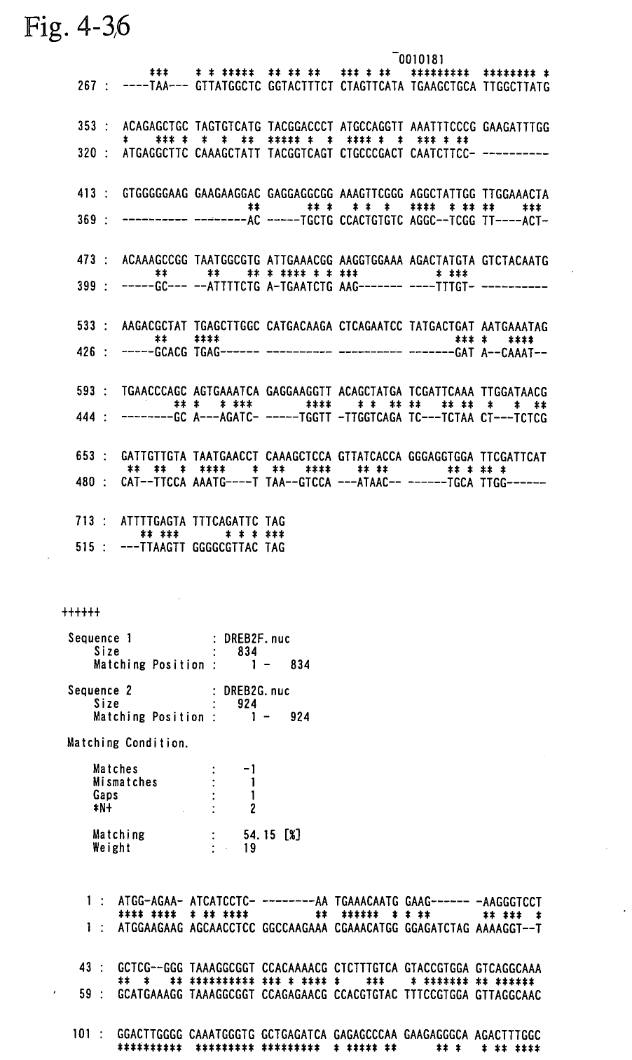

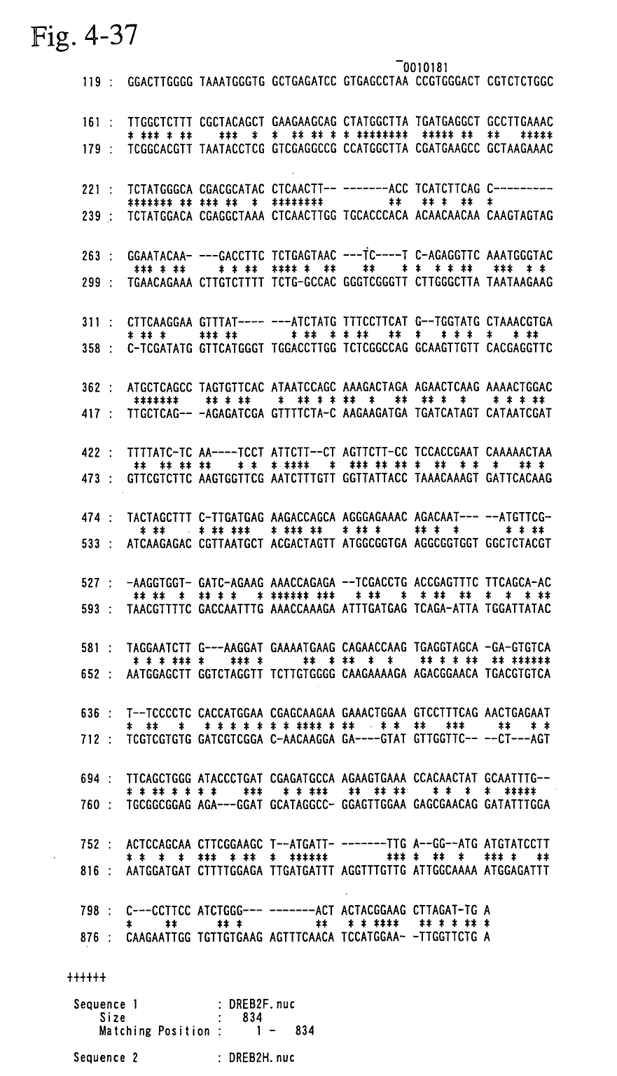

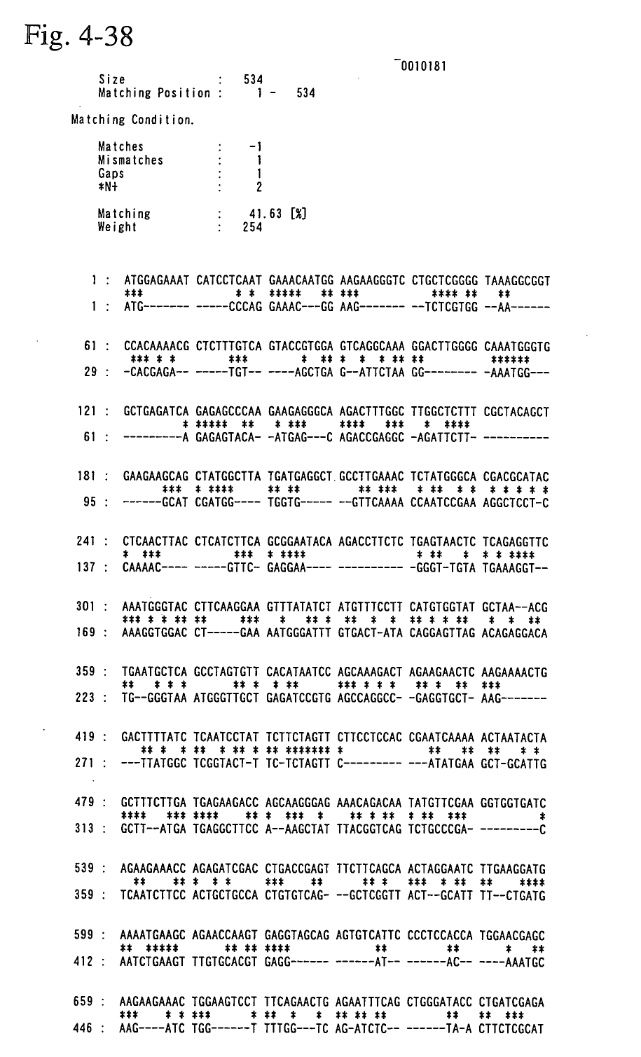

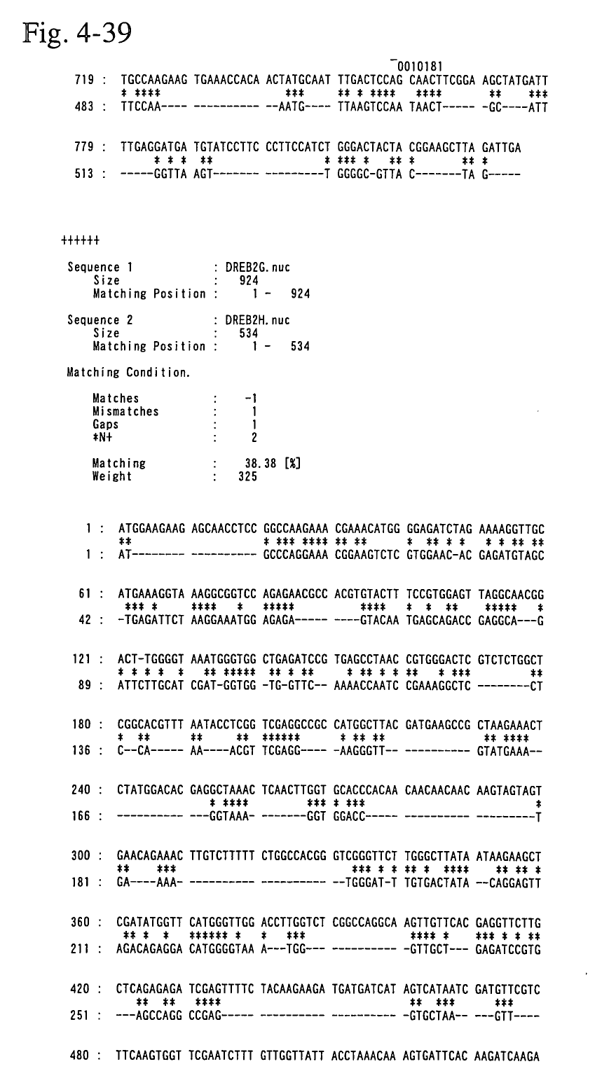

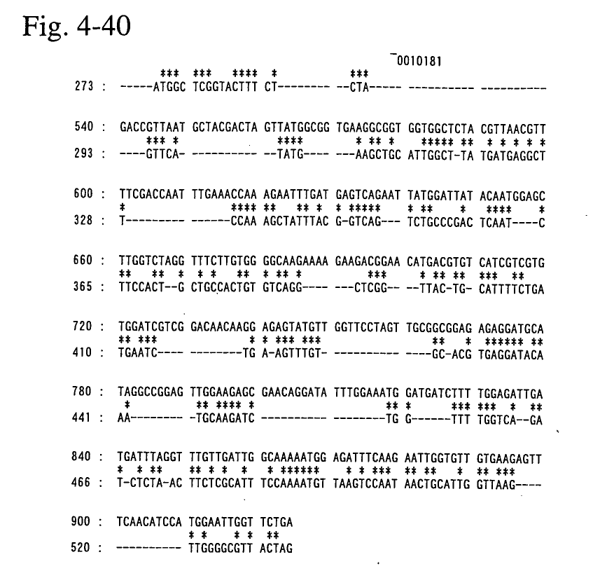

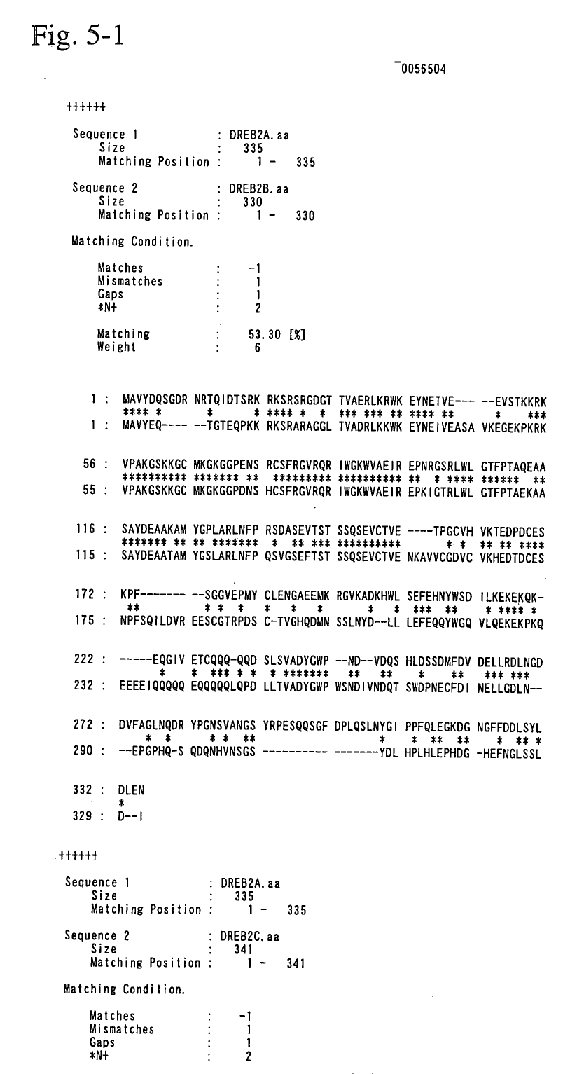

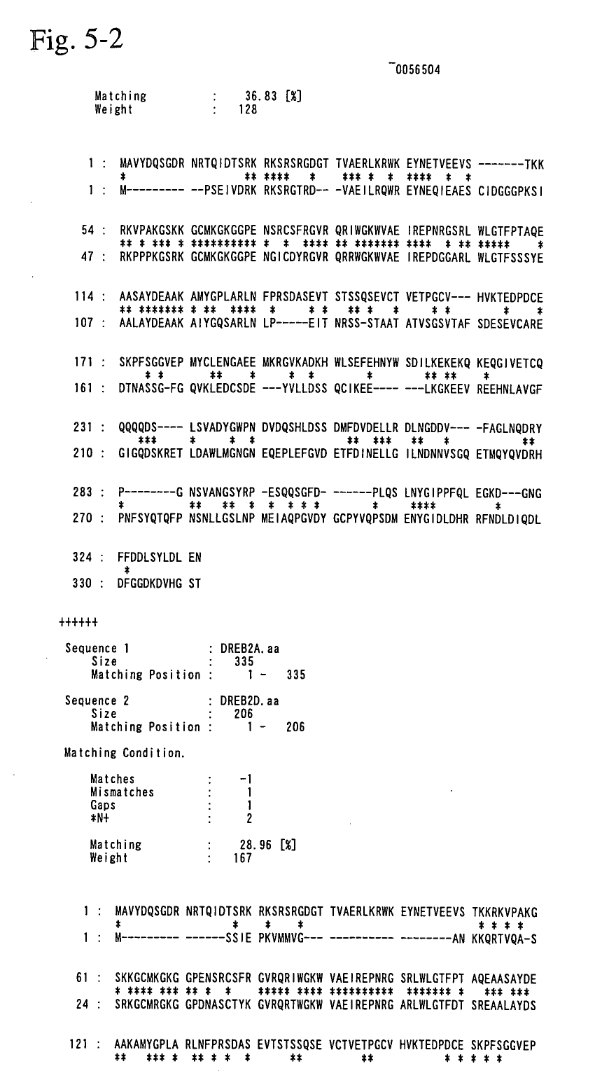

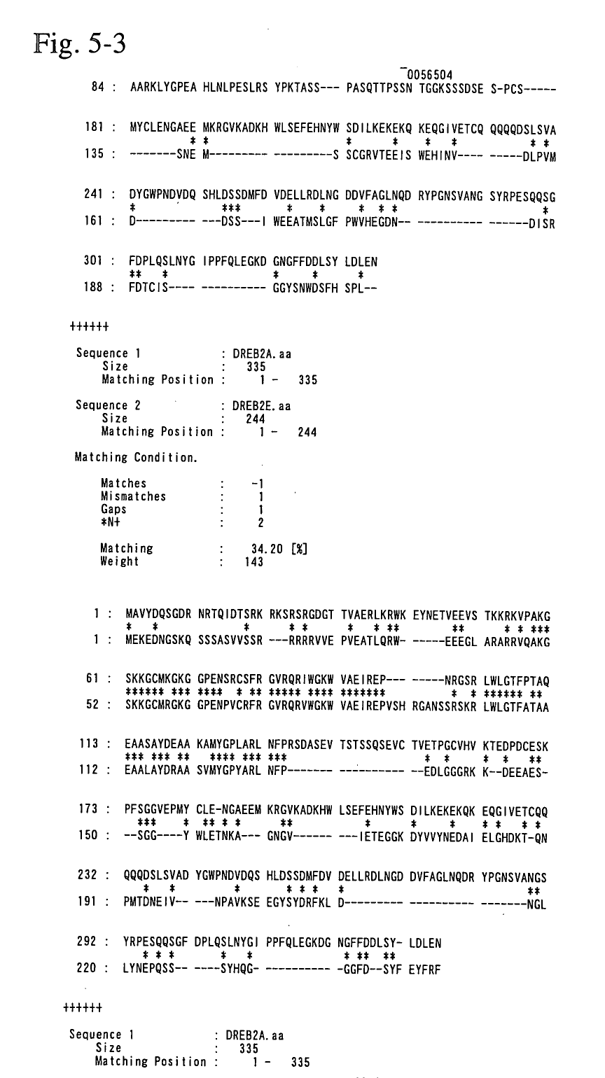

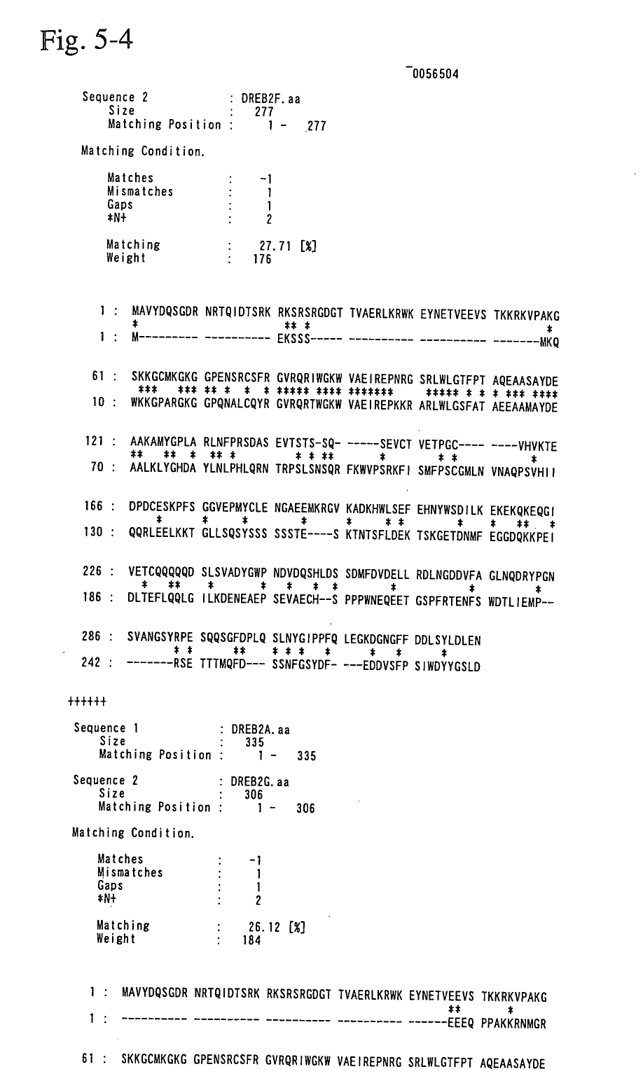

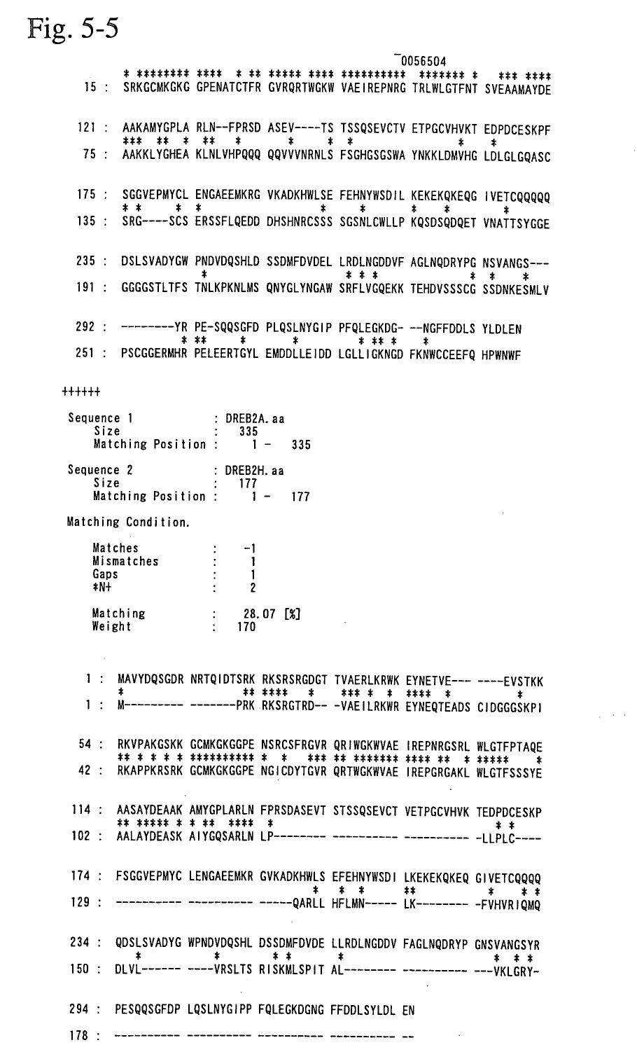

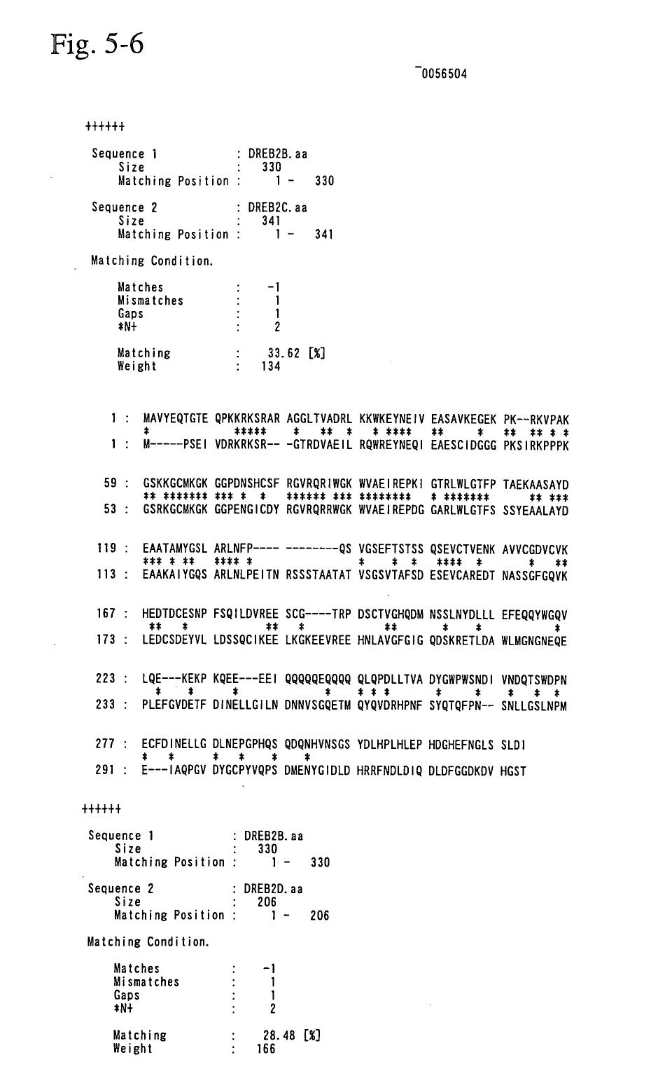

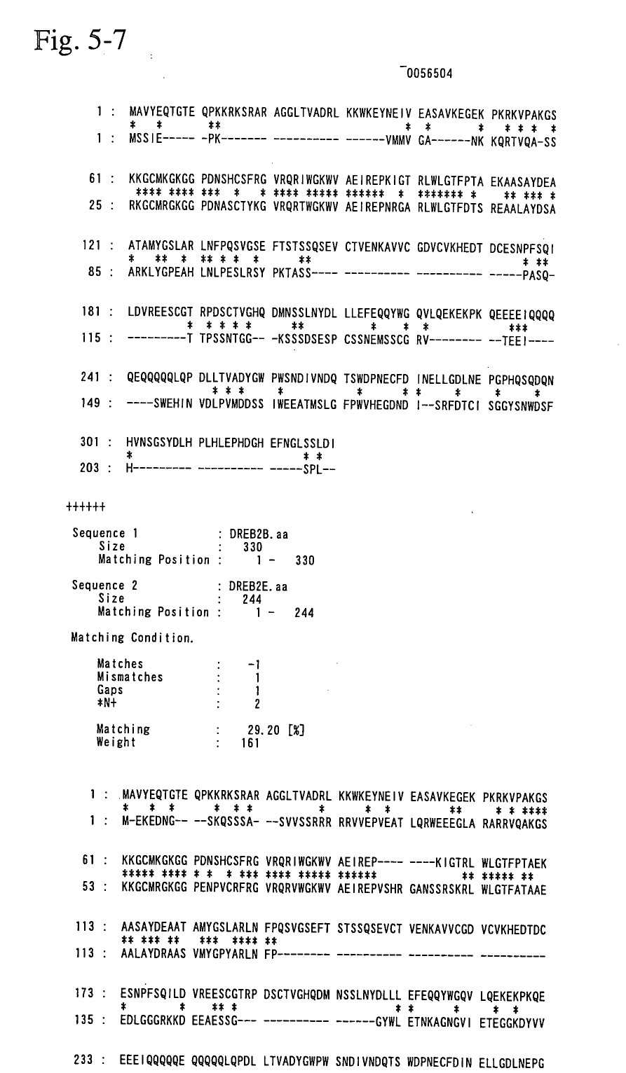

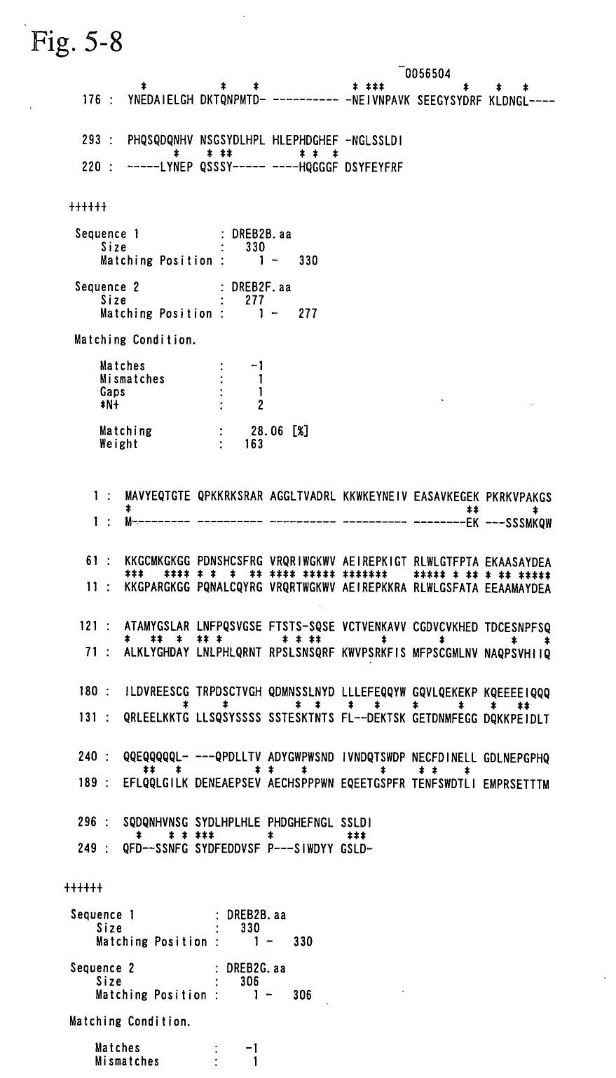

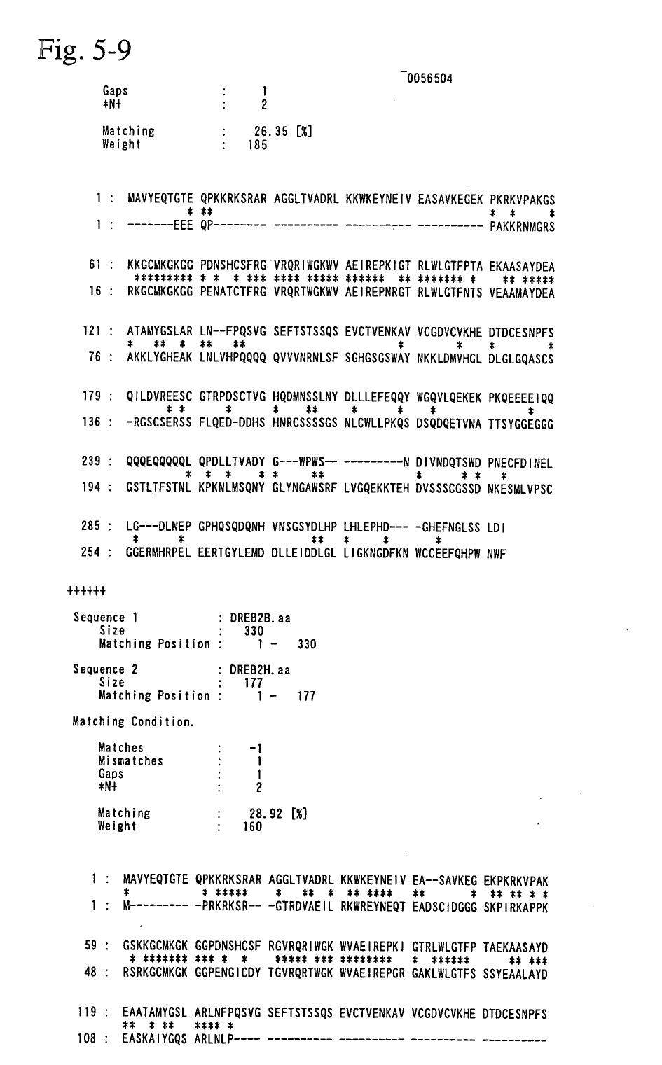

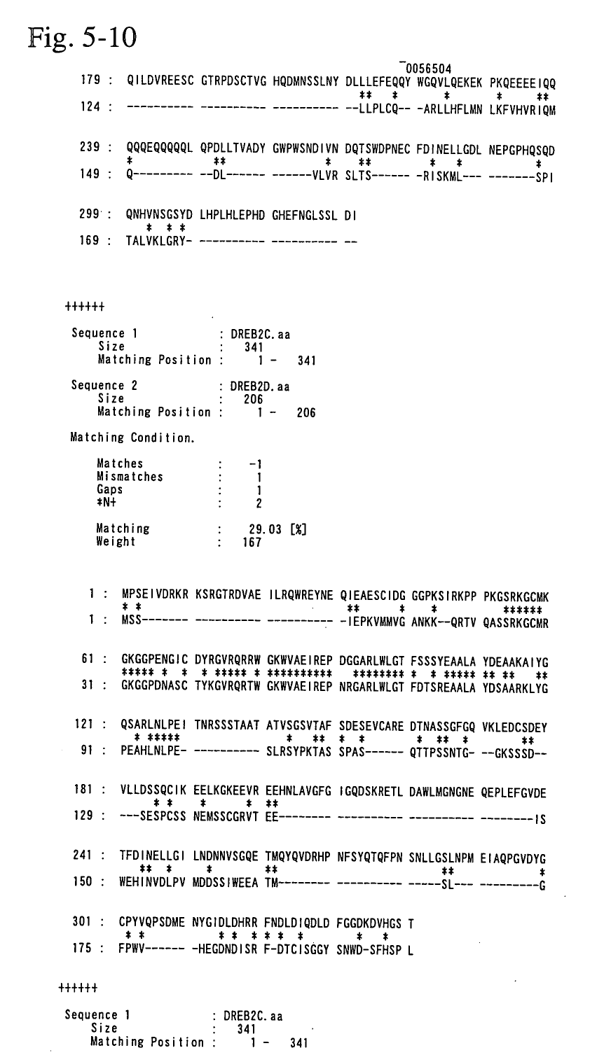

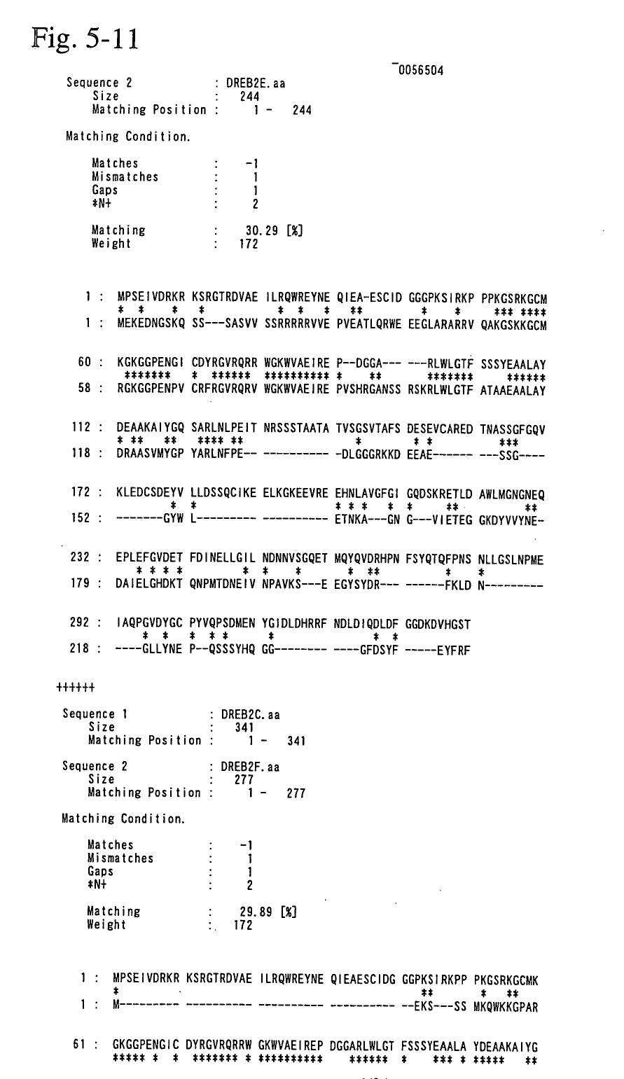

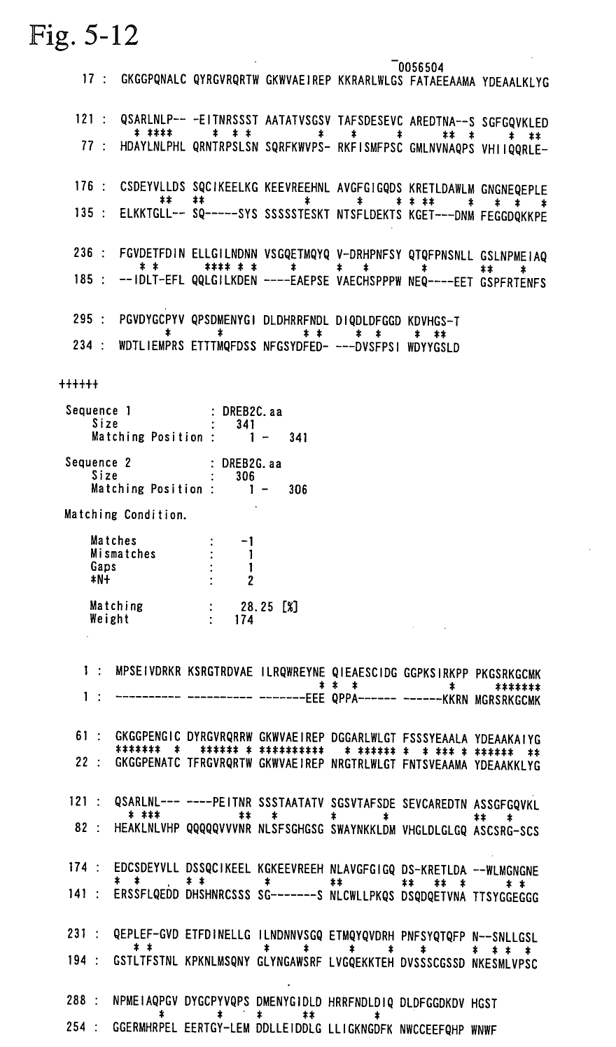

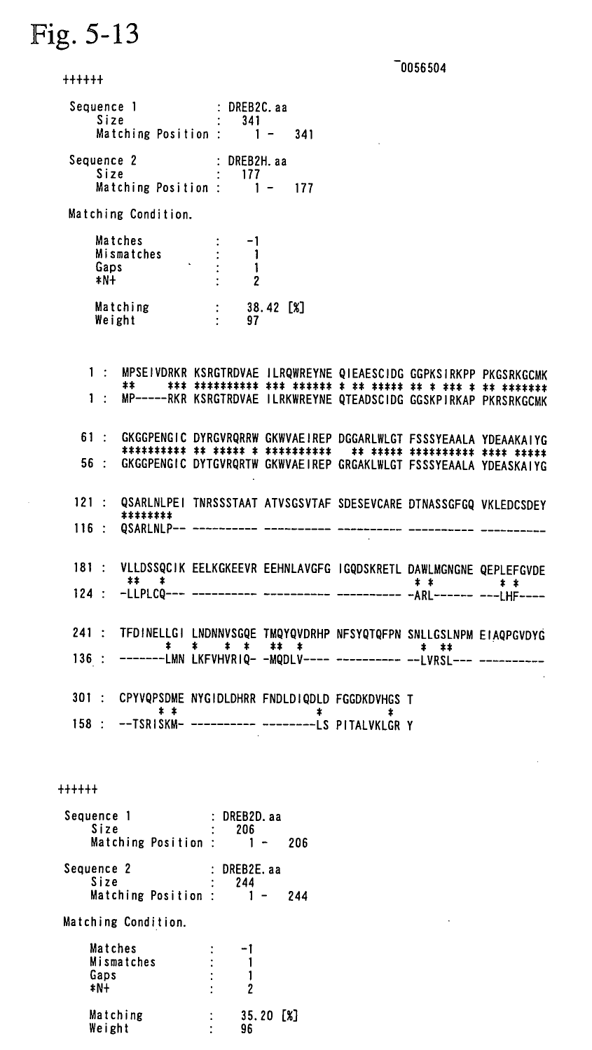

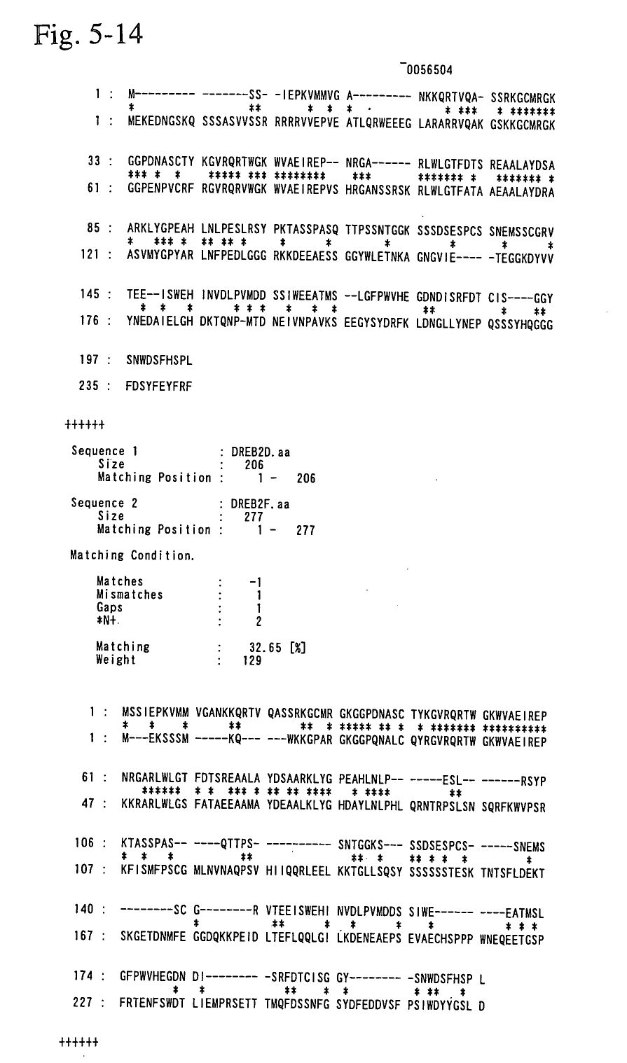

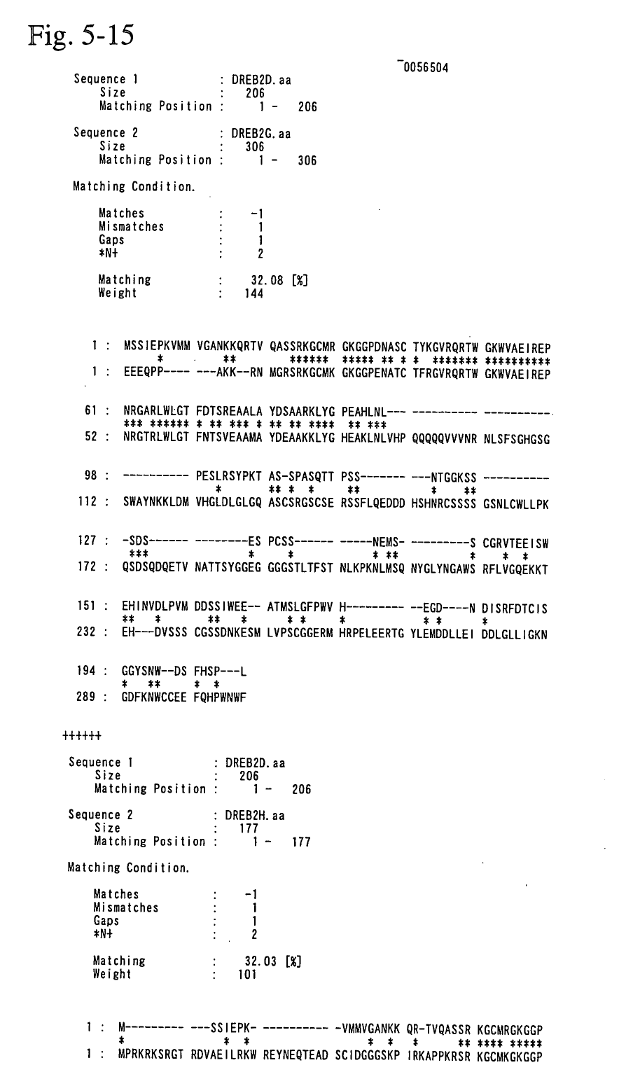

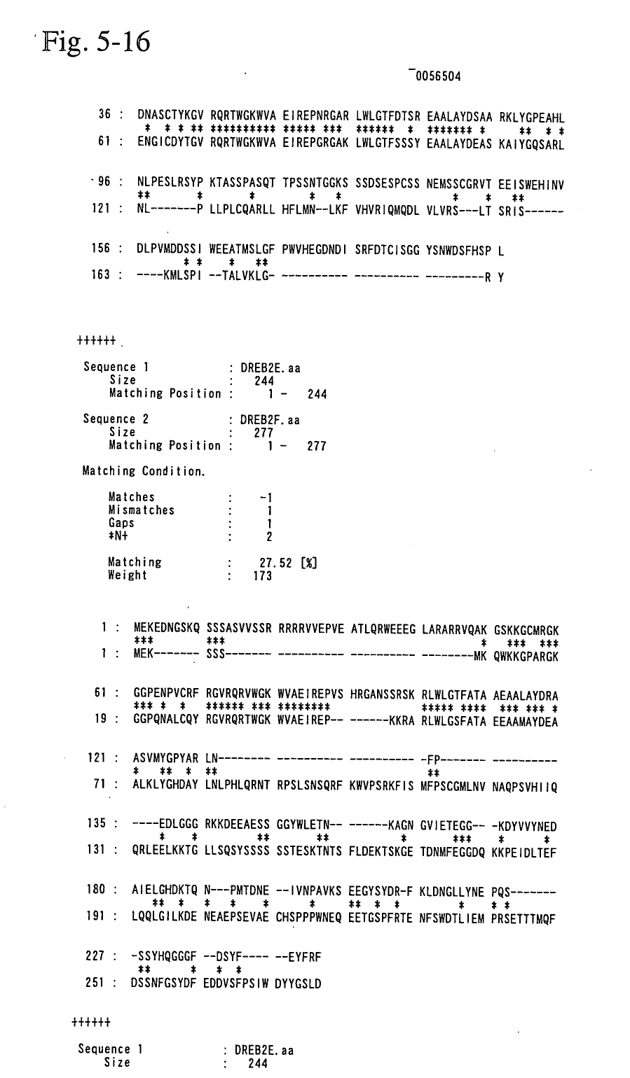

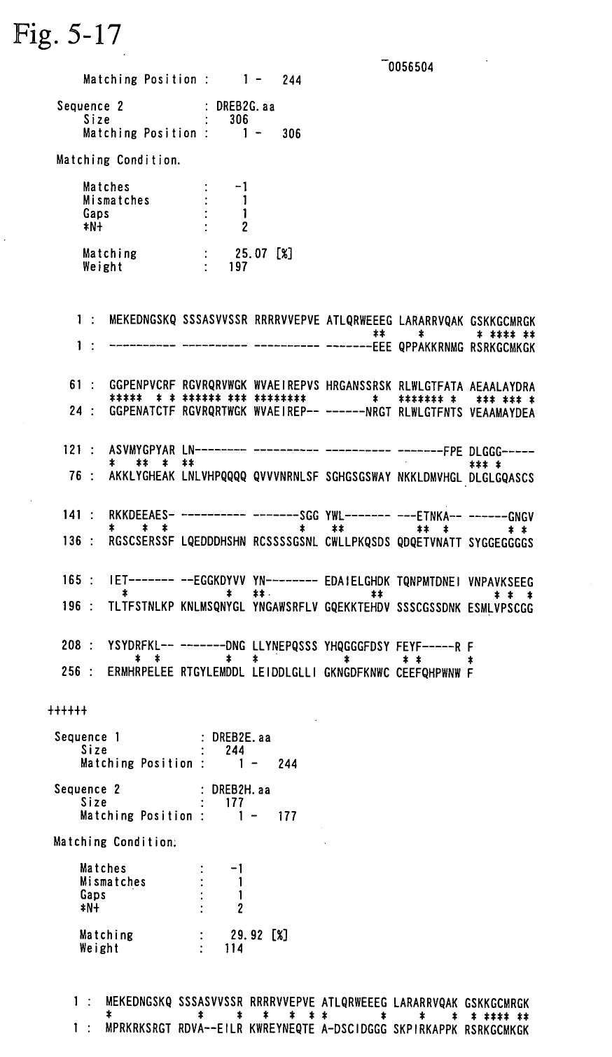

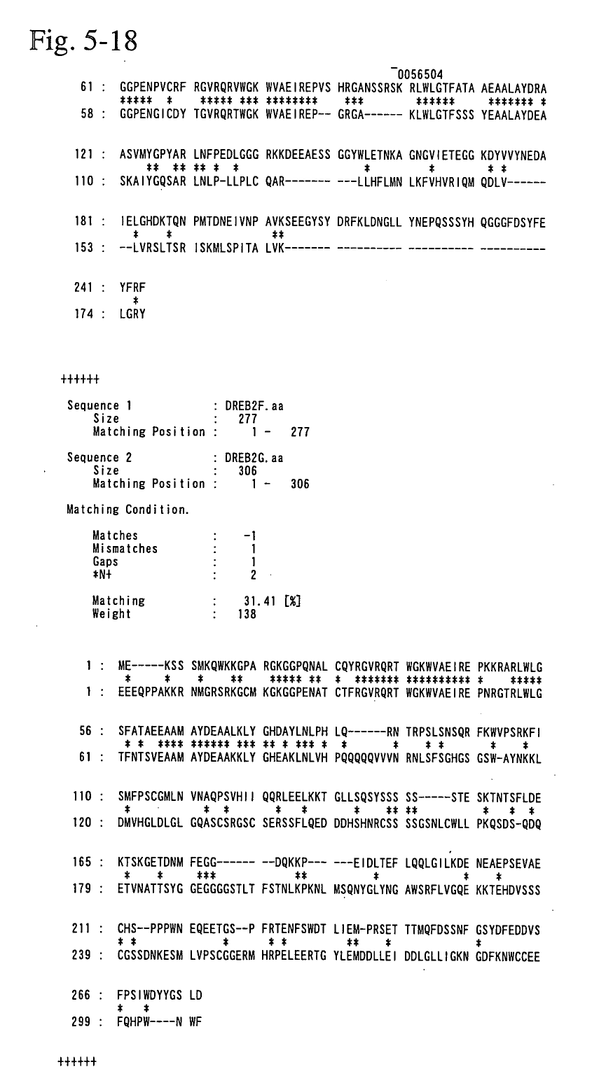

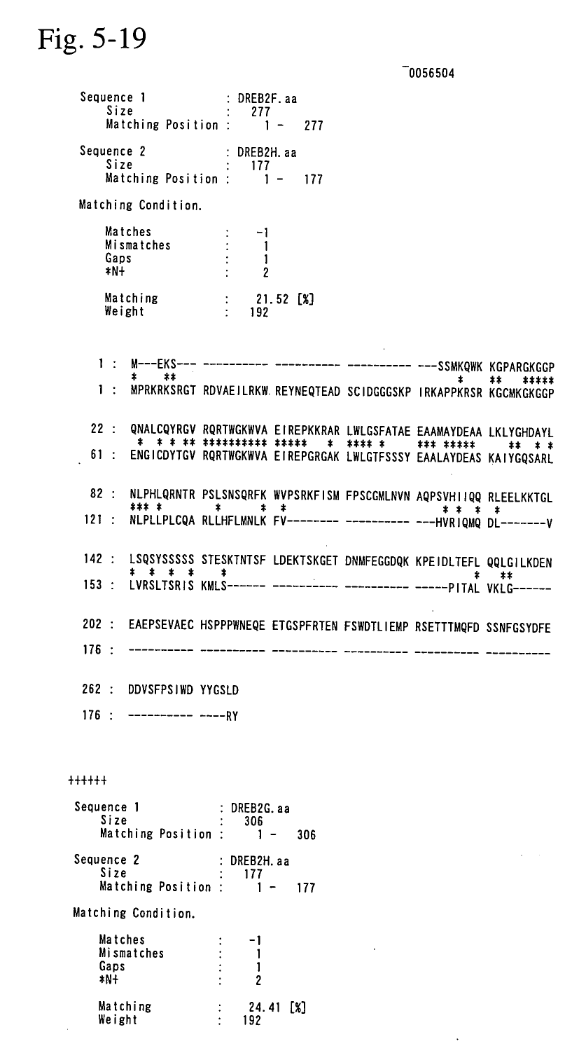

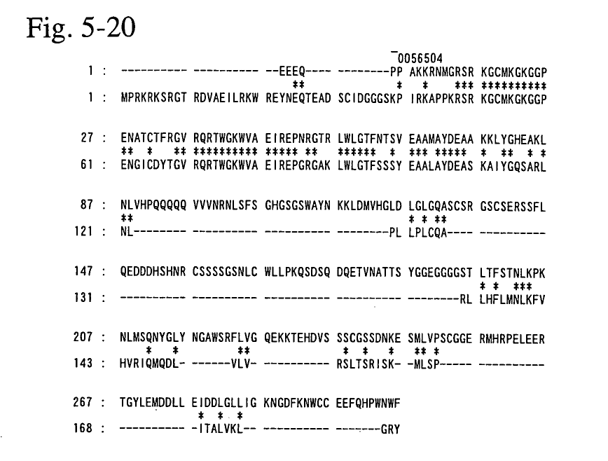

Furthermore, these genes are disclosed in JP Patent No. 3178672, and can be obtained according to the description given in this publication. In addition, the nucleotide sequences of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene are shown respectively in SEQ ID NOS: 1, 3, 5, 7, 9, 11, 13, 15, 17, 19, 21, 23, 25, and 27. In addition, the amino acid sequences of the proteins respectively encoded by DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene are respectively shown in SEQ ID NOS: 2, 4, 6, 8, 10, 12, 14, 16, 18, 20, 22, 24, 26, and 28. Furthermore, a recombinant vector containing DREB1A or DREB2A gene has been introduced into the Escherichia coli K-12 strain, and Escherichia coli containing DREB1A gene and Escherichia coli containing DREB2A gene were respectively deposited under accession nos. FERM P-16936 and FERM P-16937 with the International Patent Organism Depositary, the National Institute of Advanced Industrial Science and Technology (Tsukuba Central 6, 1-1-1 Higashi, Tsukuba, Ibaraki, Japan) on Aug. 11, 1998. Furthermore, 1 to 1 alignment, common sequences, and homology % at the nucleotide sequence level between DREB1A as a standard and each one of DREB1B to DREB1F are shown in FIG. 2; 1 to 1 alignment, common sequences and homology % at the amino acid sequence level between DREB1A as a standard and each one of DREB1B to DREB1F are shown in FIG. 3; 1 to 1 alignment, common sequences and homology % at the nucleotide sequence level between DREB2A as a standard and each one of DREB2B to DREB2H are shown in FIG. 4; 1 to 1 alignment, common sequences, and homology % at the amino acid sequence level between DREB2A as a standard and each one of DREB2B to DREB2H are shown in FIG. 5. For this alignment, GENETYX-MAC version 12.0.0 was used as analysis software. In addition, the analyses of the nucleotide sequences, amino acid sequences and the expression of DREB1D to DREB1F, and DREB2C to DREB2H are described in Biochem. Biophys. Res. Comm, 290: 998-1009 (2002). To obtain the DREB gene of the invention of this application, this literature can be referred to.

According to sequence comparison at the nucleotide sequence level among DREB1A to DREB1F genes in FIG. 2, the lowest homology between DREB1A and each one of DREB1B to DREB1F is 54.7%. In addition, among DREB1B to DREB1F, the lowest homology is 51.2% between DREB1D and DREB1E. Furthermore among DREB1A to DREB1F, many common sequences are present in a sequence region corresponding to a sequence ranging approximately from nucleotide positions 100 to 400 of DREB1A. The lowest homology of a region corresponding to a nucleotide sequence ranging from positions 100 to 400 of DREB1A is approximately 65% between DREB1D and DREB1E.

Therefore, a DNA that comprises a nucleotide sequence having 50% or more homology with the nucleotide sequence of any one of DREB1A to DREB1F and is of a gene of DREB1 family can be used as the DNA of the present invention encoding a transcription factor having functions to bind to a dehydration responsive element (DRE) and activate the transcription of a gene located downstream of the DRE. Among these DNAs, in particular, a DNA having a nucleotide sequence region that shares high homology with a nucleotide sequence region ranging from positions 100 to 400 of DREB1A, or with a nucleotide sequence region corresponding to the nucleotide sequence region ranging from positions 100 to 400 of DREB1A when the nucleotide sequence of any one of DREB1B to DREB1F is aligned with the nucleotide sequence of DREB1A by the above method can be appropriately used. Specifically, such a DNA having a region that shares at least 60% and preferably 65% or more homology with that of any one of DREB1A to DREB1F can be used as the DNA of the present invention encoding a transcription factor having functions to bind to a dehydration responsive element (DRE) and activate the transcription of a gene located downstream of the DRE. Furthermore, a DNA containing at least the above nucleotide sequence region can also be used as the DNA of the present invention encoding a transcription factor having functions to bind to a dehydration responsive element (DRE) and activate the transcription of a gene located downstream of the DRE.

According to sequence comparison at the amino acid level among DREB1A to DREB1F proteins in FIG. 2, the lowest homology between DREB1A and each one of DREB1B to DREB1F is 43.9%. In addition, among DREB1B to DREB 1F, the lowest homology is 41.9% between DREB1D and DREB1E.

Hence, a DNA encoding a protein that belongs to the DREB1 family and comprises an amino acid sequence having 40% or more homology with the amino acid sequence of any one of DREB1A to DREB1F can be used as the DNA of the present invention encoding a transcription factor having functions to bind to a dehydration responsive element (DRE) and activate the transcription of a gene located downstream of the DRE. Among these DNAs, in particular a DNA encoding a protein having an amino acid sequence region that shares high homology with an amino acid sequence region ranging approximately from amino acid positions 31 to 120 of DREB1A protein or with an amino acid sequence region corresponding to the amino acid sequence region ranging from amino acid positions 31 to 120 of DREB1A when the amino acid sequence of any one of DREB1B to DREB1F proteins is aligned with the amino acid sequence of DREB1A protein by the above method can be appropriately used. Specifically, such a DNA encoding a protein having the region that shares at least 60% and preferably 70% or more homology with that of any one of DREB1A to DREB1F can be used as the DNA of the present invention encoding a transcription factor having functions to bind to a dehydration responsive element (DRE) and activate the transcription of a gene located downstream of the DRE. Furthermore, a DNA encoding a protein containing at least the above amino acid sequence region can also be used as the DNA of the present invention encoding a transcription factor having functions to bind to a dehydration responsive element (DRE) and activate the transcription of a gene located downstream of the DRE. Furthermore, among the amino acid sequences of DREB1A to DREB1F proteins, an amino acid sequence (MAARAHDVA) ranging from positions 85 to 93 and an amino acid sequence (ALRGRSACLNF) ranging from positions 95 to 105 of DREB1A protein are common sequences of DREB1A to DREB1F proteins. A DNA encoding a protein having the entirety of both common sequences, or a sequence derived from the common sequences by substitution, deletion, or addition of 1 or several amino acids can also be used as the DNA of the present invention encoding a transcription factor having functions to bind to a dehydration responsive element (DRE) and activate the transcription of a gene located downstream of the DRE.

According to sequence comparison at the nucleotide sequence level among DREB2A to DREB2H genes in FIG. 4, the lowest homology between DREB2A and each one of DREB2B to DREB2H is 39.4%. In addition, among DREB2B to DREB2H, the lowest homology is 38.4% between DREB2G and DREB2H. Furthermore, among DREB2A to DREB2H, many common sequences are present in a sequence region ranging approximately from nucleotide positions 180 to 400.

Hence, a DNA that comprises a nucleotide sequence having 50% or more homology with the nucleotide sequence of any one of DREB2A to DREB2H and is of a gene of DREB2 family can be used as the DNA of the present invention encoding a transcription factor having functions to bind to a dehydration responsive element (DRE) and activate the transcription of a gene located downstream of the DRE. Among these DNAs, in particular a DNA having a nucleotide sequence region that shares high homology with a nucleotide sequence region ranging from positions 180 to 400 of DREB2A or with a nucleotide sequence region corresponding to the nucleotide sequence region ranging from positions 180 to 400 of DREB2A when the nucleotide sequence of any one of DREB2B to DREB2H is aligned with the nucleotide sequence of DREB2A by the above method can be appropriately used. Specifically, such a DNA having a region that shares at least 40% and preferably 50% or more homology with that of any one of DREB2A to DREB2H can be used as the DNA of the present invention encoding a transcription factor having functions to bind to a dehydration responsive element (DRE) and activate the transcription of a gene located downstream of the DRE. Furthermore, a DNA containing at least the above nucleotide sequence region can also be used as the DNA of the present invention encoding a transcription factor having functions to bind to a dehydration responsive element (DRE) and activate the transcription of a gene located downstream of the DRE.

According to sequence comparison at the amino acid sequence level among DREB2A to DREB2H proteins in FIG. 5, the lowest homology between DREB2A and each one of DREB2B to DREB2H is 26.1%. In addition, among DREB2B to DREB2H, the lowest homology is 21.5% between DREB2F and DREB2G.

Hence, a DNA encoding a protein belonging to the DREB2 family comprising an amino acid sequence having 20% or more homology with the amino acid sequence of any one of DREB2A to DREB2H can be used as the DNA of the present invention encoding a transcription factor having functions to bind to a dehydration responsive element (DRE) and activate the transcription of a gene located downstream of the DRE. Among these DNAs, in particular a DNA encoding a protein having an amino acid sequence region that shares high homology with an amino acid sequence region ranging approximately from amino acid positions 61 to 130 of DREB2A protein, or with an amino acid sequence region corresponding to the amino acid sequence region ranging from amino acid positions 61 to 130 of DREB2A when the amino acid sequence of any one of DREB2B to DREB2H proteins is aligned with the amino acid sequence of DREB2A protein by the above method can be appropriately used. Specifically, such a DNA encoding a protein having a region that shares at least 20% and preferably 30% or more homology with that of any one of DREB2A to DREB2H can be used as the DNA of the present invention encoding a transcription factor having functions to bind to a dehydration responsive element (DRE) and activate the transcription of a gene located downstream of the DRE. Furthermore, a DNA encoding a protein containing at least the above amino acid sequence region can also be used as the DNA of the present invention encoding a transcription factor having functions to bind to a dehydration responsive element (DRE) and activate the transcription of a gene located downstream of the DRE. Furthermore, among the amino acid sequences of DREB2A to DREB2H proteins, an amino acid sequence (WGKWVAEIREP) ranging from positions 88 to 98 of DREB2A protein is a common sequence of DREB2A to DREB2H proteins. A DNA encoding a protein having the entire common sequence region or a sequence derived from the common sequence by substitution, deletion, or addition of 1 or several amino acids can also be used as the DNA of the present invention encoding a transcription factor having functions to bind to a dehydration responsive element (DRE) and activate the transcription of a gene located downstream of the DRE.

In addition, “family” means molecules belonging to a group of molecules relating to DREB1A to F and DREB2A to H molecular-systematically, and having a specific homology at the amino acid sequence level therewith, and includes those other than DREB1A to F and DREB2A to H.

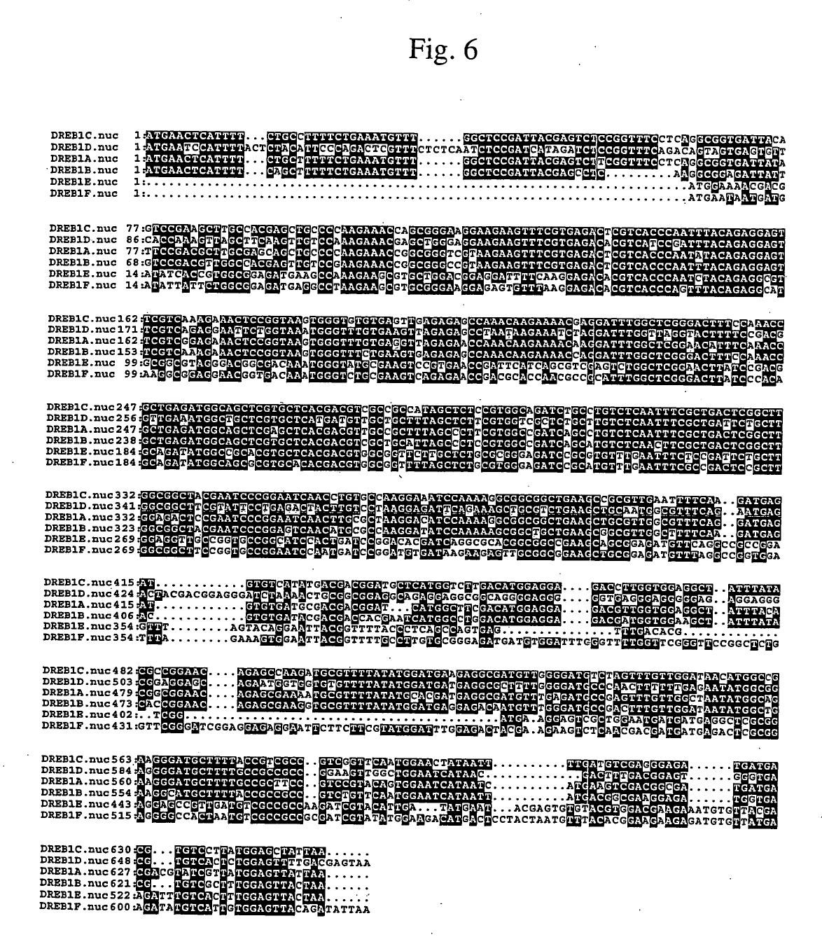

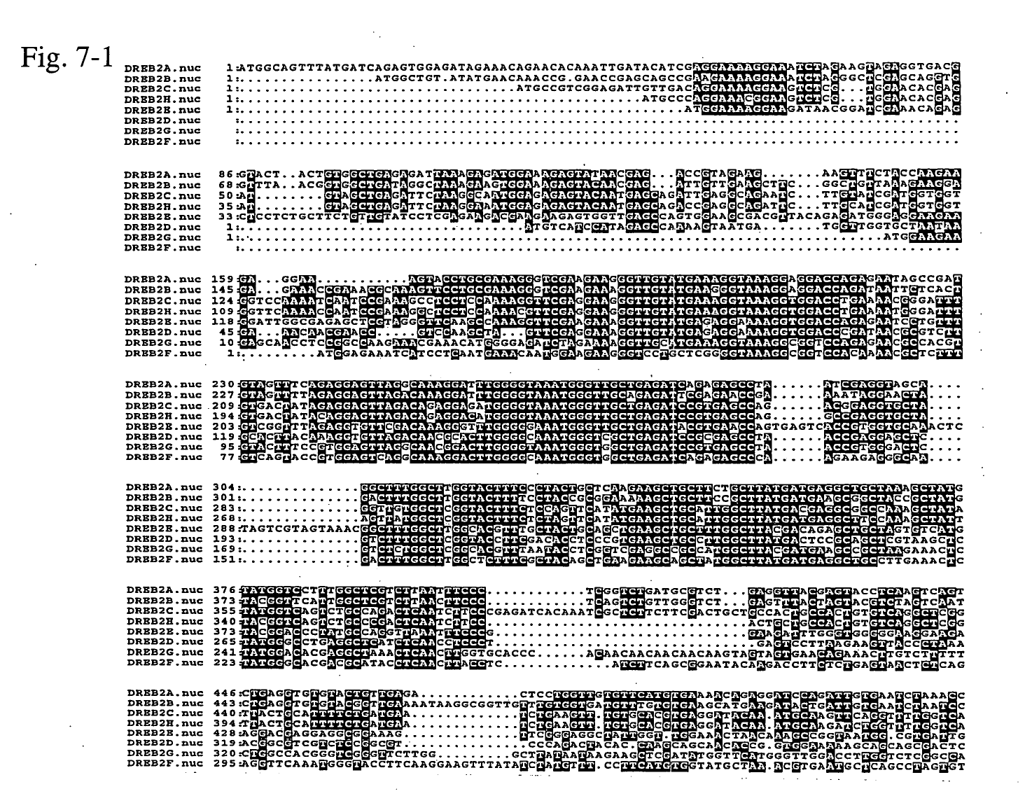

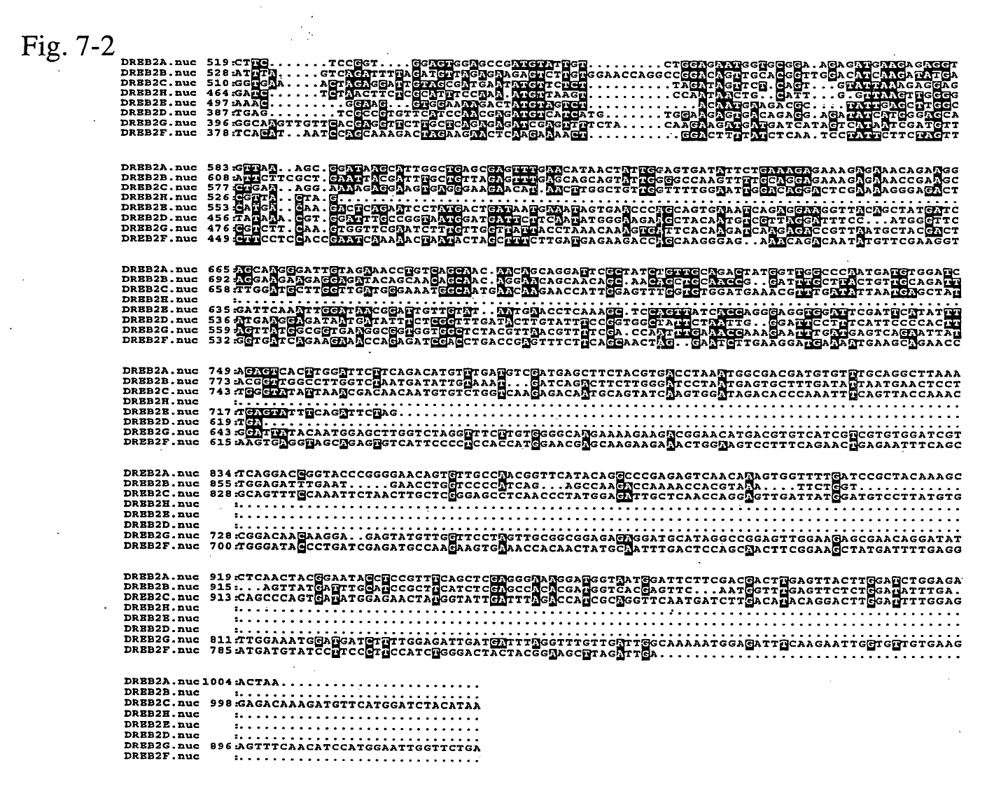

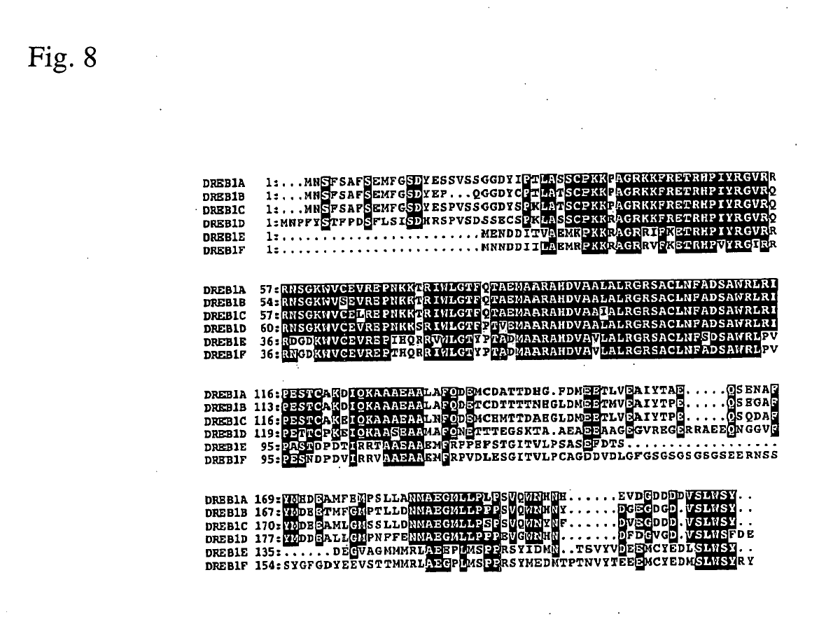

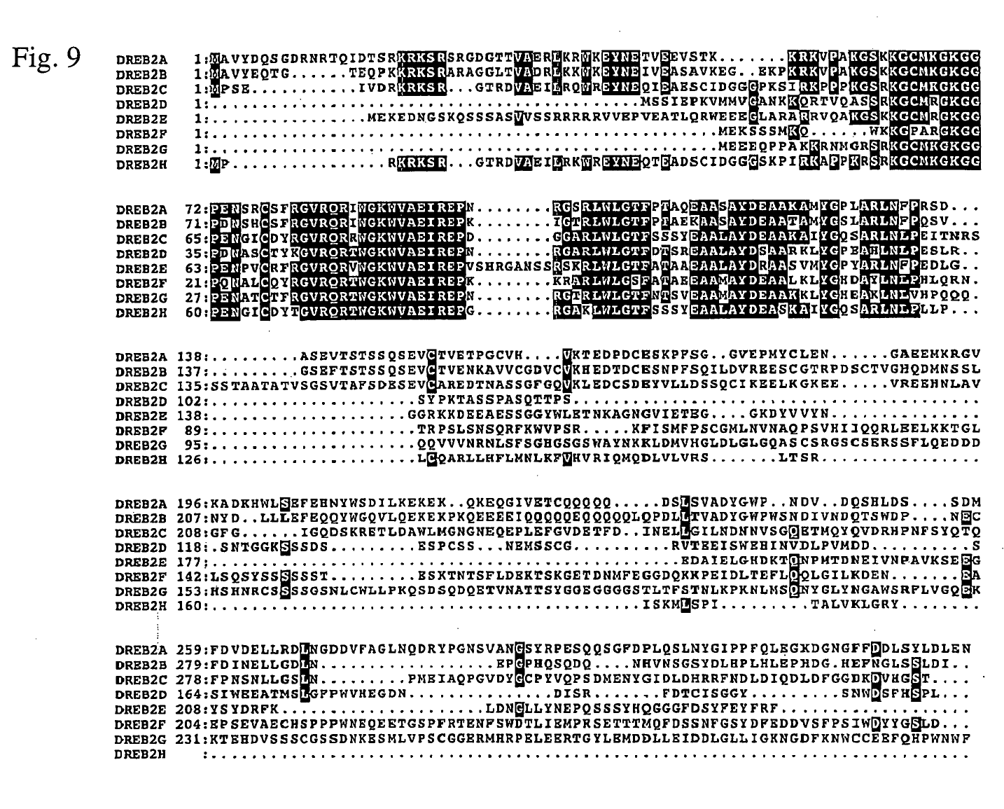

Furthermore, FIG. 6 shows alignment at the nucleotide sequence level between DREB1A as a standard and each one of DREB1B to DREB1F, FIG. 7 shows alignment at the nucleotide sequence level between DREB2A as a standard and each one of DREB2B to DREB2H, FIG. 8 shows alignment at the amino acid sequence level between DREB1A as a standard and each one of DREB1B to DREB1F, and FIG. 9 shows alignment at the amino acid sequence level between DREB2A as a standard and each one of DREB2B to DREB2H. A DNA comprising any one of the DNAs hybridizing under stringent conditions to DNAs that consist of each common nucleotide sequence when the above DREB1A or DREB2A is used as a standard, a degenerate mutant of the sequence, a sequence having 80% or more homology with such sequence, and a DNA complementary to the sequence can be used as the DNA of the present invention encoding a transcription factor having functions to bind to a dehydration responsive element (DRE) and activate the transcription of a gene located downstream of the DRE. Furthermore, a DNA encoding a protein having an amino acid sequence of any one of the common amino acid sequences when the above DREB1A or DREB2A is used as a standard, or an amino acid sequence derived from the common amino acid sequence by substitution, deletion, addition, or insertion of one or several amino acids, can also be used as the DNA of the present invention encoding a transcription factor having functions to bind to a dehydration responsive element (DRE) and activate the transcription of a gene located downstream of the DRE.

Common sequences at the amino acid level among DREB1A to 1F, common sequences at the amino acid level among DREB2A to 2H, common sequences at the nucleotide level among DREB1A to 1F, and common sequences at the nucleotide sequence level among DREB2A to 2H are shown below.

*DREB1A to 1F Amino Acid Level:

In DREB1A, an amino acid at position 30 is A, amino acids at positions 34 to 36 are P, K, and K, respectively, amino acids at positions 38 to 40 are A, G and R, respectively, an amino acid at position 43 is F, amino acids at positions 45 to 49 are E, T, R, H, and P, respectively, amino acids at positions 51 to 53 are V, R and G, respectively, an amino acid at position 55 is R, an amino acid at position 57 is R, amino acids at positions 61 to 63 are K, W, and V, respectively, an amino acid at position 65 is E, amino acids at positions 67 to 69 are R, E, and P, respectively, an amino acid at position 74 is R, amino acids at positions 76 to 79 are W, L, G and T, respectively, an amino acid at position 82 is T, amino acids at positions 85 to 93 are M, A, A, R, A, H, D, V, and A, respectively, amino acids at positions 96 to 106 are A, L, R, G, R, S, A, C, L, N, and F, respectively, amino acids at positions 108 to 113 are D, S, A, W, R, and L, respectively, an amino acid at position 116 is P, an amino acid at position 124 is I, an amino acid at position 128 is A, amino acids at positions 130 to 132 are E, A, and A, respectively, an amino acid at position 135 is F, amino acids at positions 186 and 187 are A and E, respectively, an amino acid at position 190 is L, an amino acid at position 194 is P, and amino acids at positions 212 to 215 are S, L, W, and S, respectively.

*DREB2A to 2H Amino Acid Level:

In DREB2A, amino acids at positions 63 and 64 are K and G, respectively, amino acids at positions 68 to 71 are G, K, G, and G, respectively, an amino acid at position 72 is P, an amino acid at position 74 is N, amino acid at position 77 is C, amino acids at positions 81 to 85 are G, V, R, O, and R, respectively, amino acids at positions 87 to 97 are W, G, K, W, V, A, E, I, R, E, and P, respectively, amino acids at positions 103 to 106 are L, W, L, and G, respectively, an amino acid at position 108 is F, amino acids at positions 114 and 115 are A and A, respectively, amino acids at positions 117 to 119 are A, Y, and D, respectively, an amino acid at position 121 is A, amino acids at positions 126 and 127 are Y and G, respectively, an amino acid at position 130 is A, and amino acids at positions 132 and 133 are L and N, respectively.

*DREB1A to 1F Nucleotide Level:

In DREB1A, a nucleotide at position 71 is A, a nucleotide at position 82 is A, a nucleotide at position 86 is T, nucleotides at positions 88 and 89 are G and C, respectively, a nucleotide at position 94 is A, both nucleotides at positions 100 and 101 are C, nucleotides at positions 103 to 107 are A, A, G, A, and A, respectively, a nucleotide at position 109 is C, nucleotides at positions 112 and 113 are G and C, respectively, both nucleotides at positions 115 and 116 are G, a nucleotide at position 119 is G, a nucleotide at position 121 is A, both nucleotides at positions 127 and 128 are T, nucleotides at positions 133 to 137 are G, A, G, A, and C, respectively, nucleotides at positions 139 to 143 are C, G, T, C, and A, respectively, both nucleotides at positions 145 and 146 are C, a nucleotide at position 149 is T, nucleotides at positions 151 to 158 are T, A, C, A, G, A, G, and G, respectively, a nucleotide at position 161 is T, a nucleotide at position 164 is G, a nucleotide at position 166 is C, nucleotides at positions 169 and 170 are A and G, respectively, a nucleotide at position 173 is A, a nucleotide at position 178 is G, both nucleotides at positions 181 and 182 are A, nucleotides at positions 184 to 188 are T, G, G, G, and T, respectively, a nucleotide at position 190 is T, nucleotides at positions 193 and 194 are G and A, respectively, a nucleotide at position 197 is T, a nucleotide at position 200 is G, nucleotides at positions 202 and 203 are G and A, respectively, both nucleotides at positions 205 and 206 are C, a nucleotide at position 208 is A, a nucleotide at position 212 is A, a nucleotide at position 215 is A, a nucleotide at position 221 is G, a nucleotide at position 224 is T, nucleotides at positions 226 to 228 are T, G, and G, respectively, a nucleotide at position 230 is T, both nucleotides at positions 232 and 233 are G, nucleotides at positions 235 and 236 are A and C, respectively, a nucleotide at position 238 is T, a nucleotide at position 241 is C, nucleotides at positions 244 and 245 are A and C, respectively, a nucleotide at position 247 is G, nucleotides at positions 250 and 251 are G and A, respectively, nucleotides at positions 253 to 257 are A, T, G, G, and C, respectively, nucleotides at positions 259 and 260 are G and C, respectively, nucleotides at positions 262 and 263 are C and G, respectively, nucleotides at positions 265 and 266 are G and C, respectively, nucleotides at positions 268 and 269 are C and A, respectively, nucleotides at positions 271 and 272 are G and A, respectively, nucleotides at positions 274 and 275 are G and T, respectively, nucleotides at positions 277 and 278 are G and C, respectively, a nucleotide at position 280 is G, a nucleotide at position 284 is T, nucleotides at positions 286 and 287 are G and C, respectively, nucleotides at positions 289 and 290 are C and T, respectively, nucleotides at positions 292 and 293 are C and G, respectively, both nucleotides at positions 295 and 296 are G, a nucleotide at position 299 is G, nucleotides at positions 301 and 302 are T and C, respectively, nucleotides at positions 304 and 305 are G and C, respectively, nucleotides at positions 307 to 309 are T, G, and T, respectively, a nucleotide at position 311 is T, both nucleotides at positions 313 and 314 are A, nucleotides at positions 316 to 318 are T, T, and C, respectively, a nucleotide at position 320 is C, nucleotides at positions 322 and 323 are G and A, respectively, nucleotides at positions 325 and 326 are T and C, respectively, nucleotides at positions 328 to 333 are G, C, T, T, G, and G, respectively, a nucleotide at position 335 is G, a nucleotide at position 338 is T, a nucleotide at position 340 is C, a nucleotide at position 344 is T, both nucleotides at positions 346 and 347 are C, a nucleotide at position 349 is G, a nucleotide at position 353 is C, a nucleotide at position 355 is A, a nucleotide at position 362 is C, a nucleotide at position 365 is A, nucleotides at positions 370 and 371 are A and T, respectively, nucleotides at positions 382 and 383 are G and C, respectively, a nucleotide at position 386 is C, nucleotides at positions 388 to 392 are G, A, A, G, and C, respectively, nucleotides at positions 394 and 395 are G and C, respectively, a nucleotide at position 399 is G, both nucleotides at positions 403 and 404 are T, a nucleotide at position 412 is G, nucleotides at positions 428 and 429 are C and G, respectively, a nucleotide at position 439 is G, a nucleotide at position 445 is G, a nucleotide at position 462 is G, both nucleotides at positions 483 and 484 are G, a nucleotide at position 529 is G, a nucleotide at position 533 is T, a nucleotide at position 536 is C, a nucleotide at position 545 is T, a nucleotide at position 550 is A, a nucleotide at position 554 is T, nucleotides at positions 556 and 557 are G and C, respectively, nucleotides at positions 559 and 560 are G and A, respectively, a nucleotide at position 562 is G, a nucleotide at position 569 is T, a nucleotide at position 572 is T, nucleotides at positions 575 and 576 are C and G, respectively, both nucleotides at positions 580 and 581 are C, a nucleotide at position 582 is G, nucleotides at positions 586 and 587 are G and T, respectively, a nucleotide at position 593 is T, nucleotides at positions 599 and 600 are G and A, respectively, a nucleotide at position 602 is A, a nucleotide at position 608 is A, nucleotides at positions 613 and 614 are G and A, respectively, a nucleotide at position 616 is G, a nucleotide at position 619 is G, nucleotides at positions 625 and 626 are G and A, respectively, a nucleotide at position 628 is G, a nucleotide at position 632 is T, nucleotides at positions 634 and 635 are T and C, respectively, a nucleotide at position 638 is T, nucleotides at positions 640 to 644 are T, G, G, A, and G, respectively, and a nucleotide at position 646 is T.

*DREB2A to 2H Nucleotide Level:

In DREB2A, a nucleotide at position 181 is T, a nucleotide at position 184 is A, both nucleotides at positions 187 and 188 are A, nucleotides at positions 190 to 192 are G, G, and T, respectively, both nucleotides at positions 202 and 203 are G, nucleotides at positions 205 to 209 are A, A, A, G, and G, respectively, both nucleotides at positions 211 and 212 are G, both nucleotides at positions 214 and 215 are C, a nucleotide at position 218 is A, both nucleotides at positions 220 and 221 are A, a nucleotide at position 229 is T, a nucleotide at position 230 is G, a nucleotide at position 235 is T, both nucleotides at positions 241 and 242 are G, nucleotides at positions 244 and 245 are G and T, respectively, a nucleotide at position 248 is G, nucleotides at positions 250 and 251 are C and A, respectively, a nucleotide at position 254 is G, nucleotides at positions 259 to 263 are T, G, G, G, and G, respectively, nucleotides at positions 265 to 272 are A, A, A, T, G, G, G, and T, respectively, nucleotides at positions 274 and 275 are G and C, respectively, nucleotides at positions 277 to 281 are G, A, G, A, and T, respectively, a nucleotide at position 284 is G, nucleotides at positions 286 and 287 are G and A, respectively, both nucleotides at positions 289 and 290 are C, a nucleotide at position 299 is G, a nucleotide at position 308 is T, nucleotides at positions 310 to 314 are T, G, G, C, and T, respectively, both nucleotides at positions 316 and 317 are G, a nucleotide at position 320 is C, both nucleotides at positions 322 and 323 are T, a nucleotide at position 328 is A, a nucleotide at position 332 is C, a nucleotide at position 338 is A, nucleotides at positions 340 and 341 are G and C, respectively, nucleotides at positions 343 and 344 are G and C, respectively, nucleotides at positions 349 to 353 are G, C, T, T, and A, respectively, nucleotides at positions 355 and 356 are G and A, respectively, nucleotides at positions 361 and 362 are G and C, respectively, a nucleotide at position 365 is C, a nucleotide at position 374 is T, nucleotides at positions 376 and 377 are T and A, respectively, both nucleotides at positions 379 and 380 are G, nucleotides at positions 388 and 389 are G and C, respectively, a nucleotide at position 395 is T, both nucleotides at positions 397 and 398 are A, a nucleotide at position 401 is A, a nucleotide at position 554 is A, and a nucleotide at position 572 is T.

As long as a protein comprising an amino acid sequence encoding each of the above various genes has functions to bind to the DRE so as to activate the transcription of a gene located downstream of the DRE, a mutant gene other than those of the DREB1 or DREB2 family encoding a protein that comprises an amino acid sequence derived from the above amino acid sequence by a mutation such as deletion, substitution, or addition of at least 1 or more amino acids (plurality of amino acids, or several amino acids) can be used in the present invention as a gene equivalent to each of the above genes.

For example, a gene encoding a protein that comprises an amino acid sequence derived from one of these amino acid sequences by substitution of at least 1, preferably 1 to 160, more preferably 1 to 40, further more preferably 1 to 20, and most preferably 1 to 5 amino acids with (an) other amino acid(s) can also be used in the present invention, as long as the protein has functions to bind to the DRE and activate the transcription of a gene located downstream of the DRE.

Moreover, a DNA that is capable of hybridizing under stringent conditions to a DNA complementary to the DNA of each of the above various genes can also be used in the present invention as a gene equivalent to each of the above genes, as long as a protein encoded by the DNA has functions to bind to the DRE so as to activate the transcription of a gene located downstream of the DRE. Such stringent conditions comprise, for example, sodium concentration between 10 mM and 300 mM, or preferably between 20 mM and 100 mM, and temperatures between 25° C. and 70° C., or preferably between 42° C. and 55° C. [Molecular Cloning (edited by Sambrook et al., (1989) Cold Spring Harbor Lab. Press, New York)].

Moreover, a mutant gene can be prepared according to a known technique such as the Kunkel method or the Gapped duplex method, or a method according thereto using, for example, a kit for mutagenesis (e.g., Mutant-K (TAKARA) and Mutant-G (TAKARA)) utilizing the site-directed mutagenesis method, or a LA PCR in vitro Mutagenesis series kit (TAKARA). Regarding the above mutagenesis methods, it is clear that persons skilled in the art can produce the above mutant genes without any special difficulties by referring to the nucleotide sequence of DREB gene to perform selection and the procedures according to the description in literature such as Molecular Cloning (edited by Sambrook et al. (1989) 15 Site-directed Mutagenesis of Cloned DNA, 15.3 to 15.113, Cold Spring Harbor Lab. Press, New York). Furthermore, regarding techniques (site-directed mutagenesis), by which substitution, deletion, insertion, or addition of one or more (1 or several or more nucleotides) nucleotides is artificially performed based on the nucleotide sequence of DREB gene, persons skilled in the art can obtain and utilize a mutant according to techniques described in Proc. Natl. Acad. Sci. U.S.A. 81 (1984) 5662-5666; WO85/00817, Nature 316 (1985) 601-605, Gene 34 (1985) 315-323; Nucleic Acids Res. 13 (1985) 4431-4442; Proc. Natl. Acad. Sci. U.S.A. 79 (1982) 6409-6413; Science 224 (1984) 1431-1433; or the like.

Furthermore, the DREB gene of the present invention also includes a nucleotide sequence (mutant) having 80% or more, preferably 90% or more, more preferably 94% or more, and most preferably 99% or more homology with the nucleotide sequence of each of the above DREB genes or each common nucleotide sequence thereof, as long as the mutant has functions to bind to the DRE and activate the transcription of a gene located downstream of the DRE. Here, such numerical values of homologies are calculated based on default parameter settings (initial settings) using a program for comparing nucleotide sequences, such as GENETYX-MAC version 12.0.0.

If such a mutant of a DNA comprising the nucleotide sequence of the DREB gene or a part thereof has activity to bind to the DRE and activate the transcription of a gene located downstream of the DRE, the mutant may be used in the present invention, and the strength of the activity is not specifically limited. Preferably, each mutant substantially has activity equivalent to the activity of the DNA comprising the nucleotide sequence or the part thereof to bind to the DRE and activate the transcription of a gene located downstream of the DRE. Here, the phrase, “substantially having activity equivalent to that of binding to the DRE and activating the transcription of a gene located downstream of the DRE” means that in the actual embodiment where the activity is utilized, activity is maintained to a degree such that almost the same use as that of the DNA or the part thereof is possible under the same conditions. In addition, the activity used herein means activity of, for example, plant cells or plants, preferably the cells or the plants of dicotyledon, more preferably the cells or the plants of chrysanthemum, and most preferably the cells or the plants of Lineker (Chrysanthemum morifolium cv. Lineker or Dendranthema grandiflorum cv. Lineker) of chrysanthemum cultivars. These activities can be measured according to the method disclosed in JP Patent No. 3178672.

Once the nucleotide sequence of DREB gene is determined, DREB gene can then be obtained by chemical synthesis, PCR using the cDNA or the genomic DNA of this gene as a template, or hybridization using a DNA fragment having the nucleotide sequence as a probe.

DREB gene is a gene encoding a protein that activates transcription. Thus, in a plant having the gene introduced therein, the thus expressed DREB protein acts so as to activate various genes, and the plant's own growth may be suppressed by the resulting increases in energy consumption or activation of metabolism. To prevent this from occurring, it is conceivable that a stress-responsive promoter be ligated upstream of DREB gene so as to cause the expression of DREB gene only when stress is provided. Examples of such a promoter are as follows:

rd29A gene promoter [Yamaguchi-Shinozaki, K. et al.: Plant Cell, 6: 251-264 (1994)], rd29B gene promoter [Yamaguchi-Shinozaki, K. et al.: Plant Cell, 6: 251-264 (1994)], rd17 gene promoter [Iwasaki, T. et al.: Plant Physiol., 115: 1287 (1997)], rd22 gene promoter [Iwasaki, T. et al.: Mol. Gen. Genet., 247: 391-398 (1995)], DREB1A gene promoter [Shinwari, Z. K,. et al.: Biochem. Biophys. Res. Corn. 250: 161-170 (1998)], cor6.6 gene promoter [Wang, H. et al.: Plant Mol. Biol. 28: 619-634 (1995)], cor15a gene promoter [Baker, S. S. et al.: Plant Mol. Biol. 24: 701-713 (1994)], erd1 gene promoter [Nakashima K. et al.: Plant J. 12: 851-861 (1997)], and kin1 gene promoter [Wang, H. et al.: Plant Mol. Biol. 28: 605-617 (1995)].

However, as long as a promoter is stress responsive and functions within a plant cell or a plant, it is not limited to the above promoters. In addition, these promoters can be obtained by a PCR amplification reaction using primers designed based on the nucleotide sequence of a DNA containing the promoter and the genomic DNA as a template. Specifically, a promoter can be obtained by amplifying by polymerase chain reaction (PCR) the promoter region (a region from the translation initiation point of rd29A gene −215 to −145) [Kazuko Yamaguchi-Shinozaki and Kazuo Shinozaki: Plant Cell 6: 251-264 (1994)] of the rd29A gene, which is one type of dehydration-stress-resistance gene. An example of a template DNA that can be used for PCR is a genomic DNA of Arabidopsis, but use is not limited thereto.

An example of a gene used in the present invention wherein DREB gene is ligated to a stress-responsive promoter is rd29A-DREB1A. This gene is derived from a plant plasmid pBI29AP: DREB1A described in Example 5 of JP Patent No. 3178672, and is a stress-resistance gene that has also been reported by Kasuga et al's report [Nature Biotech., 17 287-291 (1999)].

Also for such a promoter, in a manner similar to the case for the above DREB genes, various mutants can be used, as long as they possess promoter activity. Such a mutant can be prepared by persons skilled in the art without any special difficulties by referring to the nucleotide sequences described in literature concerning the various above promoters, as described also for the above DREB genes. Whether or not the mutant obtained as described above has activity as a promoter and whether or not the mutant substantially retains the promoter activity of a DNA containing the promoter or a part thereof can be confirmed by ligating useful DREB genes for expression within host cells according to the descriptions of the following examples, and then carrying out various forms of bioassay (e.g., in terms of salinity resistance, rooting ability, and prolonged life of cut flowers). Such methods can be appropriately conducted by persons skilled in the art.

Therefore, the above various stress-responsive promoters and various DREB genes can be appropriately combined, selected, and used according to the purpose of use in various plant cells or plants, so that activity can be confirmed.

Furthermore, a terminator ordering to terminate transcription can also be ligated downstream of DREB gene, if necessary. Examples of a terminator include a terminator derived from the cauliflower mosaic virus and a nopaline synthase gene terminator. However, examples of a terminator are not limited thereto, as long as they are known to function within a plant.

Furthermore, an intron sequence having a function to enhance gene expression, such as the intron of alcohol dehydrogenase (Adh1) of maize [Genes & Development 1: 1183-1200 (1987)], can be introduced between a promoter sequence and DREB gene, if necessary.

(2) DNA Strand for Production of Transformed Plant

To produce the transformed plant of the present invention, a DNA strand, which comprises the DNA of the present invention wherein a stress-responsive promoter and DREB gene are linked, is used. In a specific form of the DNA strand according to the present invention, for example, the DNA of the present invention having a stress-responsive promoter ligated to DREB gene may be inserted as a component into a plasmid or a phage DNA.

The DNA strand of the present invention can further contain components such as a translation enhancer, a translation termination codon, and a terminator. As a translation enhancer, a translation termination codon, or a terminator, those known can be appropriately combined and used. Examples of a translation enhancer of viral origin include the sequences of tobacco mosaic virus, alfalfa mosaic virus RNA 4, bromo mosaic virus RNA 3, potato virus X, and tobacco etch virus [Gallie et al., Nuc. Acids Res., 15 (1987) 8693-8711]. Moreover, examples of a translation enhancer of plant origin include a sequence derived from β-1,3 glucanase (Glu) of soybean [written by Isao Ishida and Norihiko Misawa, edited by Kodansha Scientific, Cell Technology, Introduction to Experimental Protocols (Saibo-ko-gaku jikken so-sa nyumon), KODANSHA, p. 119, 1992], and a sequence derived from a ferredoxin-binding subunit (PsaDb) of tobacco [Yamamoto et al., J. Biol. Chem., 270 (1995) 12466-12470]. Examples of a translation termination codon include sequences such as TAA, TAG, and TGA [described in the above-mentioned Molecular Cloning]. Examples of a terminator include the terminator of nos gene and the terminator of ocs gene [Annu. Rev. Plant Physiol. Plant Mol. Biol., 44 (1993) 985-994, “Plant genetic transformation and gene expression; a laboratory manual” as mentioned above]. Furthermore, it has been reported that activity can be enhanced by lining up and linking several 35S enhancer regions identified as transcription enhancers in a promoter [Plant Cell, 1 (1989) 141-150]. These regions can also be used as a part of the DNA strand. Each of these various components is preferably incorporated operably into the DNA strand so that each component can function depending on the properties thereof. Persons skilled in the art can appropriately carry out such operations.

The above DNA strand can be easily produced by persons skilled in the art using techniques generally used in the field of genetic engineering. Moreover, the DNA strand of the present invention is not limited to those isolated from natural supply sources, and may be an artificial construct, as long as it has the above-mentioned structure. The DNA strand can be obtained by synthesizing it according to a known and generally used method of synthesizing nucleic acids.

(3) Transformation of Plant

By the transformation of a host using the gene obtained in (1) above, and the culture or cultivation of the obtained transformant, a protein regulating the transcription of a gene located downstream of a stress-responsive element can be expressed, and a transformed plant having improved propagation efficiency of plant seedlings and improved vase life can be prepared.

The above DNA strand of the present invention after transformation can be present in microorganisms (particularly, bacteria), phage particles, or plants, while being inserted in a plasmid, a phage, or a genomic DNA. Here, examples of bacteria typically include Escherichia coli and Agrobacterium, but are not limited thereto.

In a preferred embodiment of the present invention, the DNA strand of the present invention is present in a plant in a form wherein the DNA of the present invention (promoter), a translation enhancer, a structural gene DNA, a translation termination codon, a terminator, and the like are integrally bound and this integrated combination thereof is inserted in the genome, so that the structural gene for the expression of a protein can be stably expressed in the plant.

Preferred examples of a host include cells of monocotyledons such as rice, wheat, corn, onions, lilies, and orchids, and cells of dicotyledons such as soybean, rapeseeds, tomatoes, potatoes, chrysanthemums, roses, carnations, petunias, baby's-breath, and cyclamens. Particularly preferred specific examples include the plant cells of 3 important cut flowers with large worldwide production amounts, turn volumes, and amounts of consumption: chrysanthemums, carnations and roses. Also particularly preferred are the plant cells of clones such as petunias whose production amounts, turn volumes, and amounts of consumption are growing drastically across the globe in recent years. In addition, examples of a specific plant material include vegetative points, shoot primordia, meristematic tissues, laminae, stem pieces, root pieces, tuber pieces, petiole pieces, protoplasts, calli, anthers, pollens, pollen tubes, flower stalk pieces, scape pieces, petals, and sepals.

As a method of introducing a foreign gene into a host, various methods that have been previously reported and established can be appropriately utilized. Preferred examples of such a method include a biological method using, for example, a virus or the Ti plasmid or the Ri plasmid of Agrobacterium as a vector, and a physical method involving introduction of a gene by electroporation, polyethylene glycol, particle gun, microinjection [the aforementioned “Plant genetic transformation and gene expression; a laboratory manual”], silicon nitride whisker, silicon carbide whisker [Euphytica 85 (1995) 75-80; In Vitro Cell. Dev. Biol. 31 (1995) 101-104; Plant Science 132 (1998) 31-43] or the like. Persons skilled in the art can appropriately select and use the introduction method.

Furthermore, by the regeneration of a plant cell transformed with the DNA strand of the present invention, a transformed plant wherein the introduced gene is expressed within the cell can be produced. Persons skilled in the art can easily conduct such a procedure by a generally known method of regenerating plants from plant cells. Regarding regeneration of plants from plant cells, for example, see literature such as [Manuals for Plant Cell Culture (Shokubutsu saibo-baiyo manual)], and [edited and written by Yasuyuki Yamada, Kodansha Scientific, 1984].

In general, a gene introduced into a plant is incorporated into the genome of a host plant. At this time, a phenomenon referred to as position effect is observed, wherein a different position on the genome to which a gene is introduced leads to a different expression of the transgene. The transformant wherein a transgene is expressed more strongly than others can be selected by assaying mRNA levels expressed in the host plant by the Northern method using the DNA fragment of the transgene as a probe.

The incorporation of a target gene into a transformed plant, into which the gene used in the present invention has been introduced, can be confirmed by extracting DNA from these cells and tissues according to any standard method, and detecting the introduced gene using the known PCR method or Southern analysis.

(4) Transformed Plant of the Present Invention

The present invention provides a transformed plant, which contains a gene wherein a DNA encoding a protein that binds to a stress-responsive element contained in a stress-responsive promoter and regulates the transcription of a gene located downstream of the element is ligated downstream of the stress-responsive promoter, and has improved rooting efficiency and/or prolonged vase life. Examples of a plant include monocotyledons such as rice, wheat, corn, onions, lilies, and orchids, and dicotyledons such as soybean, rapeseeds, tomatoes, potatoes, chrysanthemums, roses, carnations, petunias, baby's-breath and cyclamens. Particularly preferred specific examples include 3 important cut flowers with large worldwide production amounts, turn volumes and amounts of consumption: chrysanthemums, carnations and roses. Also particularly preferred are the clones such as petunias whose production amounts, turn volumes and amounts of consumption are growing drastically across the globe. The present invention also provides the scions of the transformed plants of the above plants having improved propagation efficiency and rooting efficiency compared with those of non-transformed plants, and the cut flowers of the transformed plants of the above plants having improved vase life (prolonged life of cut flowers) compared with those of non-transformed plants. Here “scions” mean branches, treetops, stems, leaves, and the like that are cut from plants and then planted for cutting. “Cut flowers” mean flowers cut from plants with branches and stems uncut.

(5) Scion Propagation Efficiency Test and Vase Life Test

The transformed plant of the present invention has improved efficiency of propagation using scions, rooting efficiency, and vase life (prolonged life of cut flowers) compared with the case of non-transformed plants.

The efficiency of propagation using scions, rooting efficiency, and vase life (prolonged life of cut flowers) of a transformed plant can be evaluated by measuring efficiencies under the same conditions as those employed for plant production. For example, the efficiency of propagation using scions or the rooting efficiency of chrysanthemums can be evaluated by planting scions in soil for scions and examining the growing conditions 2 to 4 weeks later, and the growth of the same can be evaluated by potting the plants and measuring the stem lengths or the like. Vase life can be evaluated by carrying out approximately 4 weeks of long-day cultivation after potting, followed by approximately 8 weeks of short-day cultivation, so as to cause the plants to flower, cutting chrysanthemums, allowing the plants to stand in the dark for 1 day, arranging them in water, and then observing their conditions thereafter. For the general cultivation methods for chrysanthemums, see “revised version of New Techniques for Cut Flower Cultivation, Chrysanthemum” (“Kiribanasaibai-no-shingijutsu, Kaitei, Kiku,” edited by Keiichi Funakoshi, SEIBUNDO SHINKOSHA, 1989).

### EFFECT OF THE INVENTION

As shown in Examples, a plant that has been transformed using a gene (stress-resistance gene) wherein a DNA encoding a protein that binds to a dehydration responsive element (DRE) and regulates the transcription of a gene located downstream of the DRE is ligated downstream of a stress-responsive promoter, has improved rooting efficiency and/or prolonged vase life compared with those of non-transformed plants. In addition, the transformed plant grows well after rooting. Hence, the method of introducing DREB gene into a plant of the present invention is useful in developing a plant having enhanced efficiency of propagation by cutting, enhanced rooting efficiency, and prolonged vase life.

### DETAILED DESCRIPTION OF THE PREFERRED EMBODIMENTS

The present invention will be described by examples below, but the present invention is not specifically limited by these examples.

### EXAMPLE 1

### Preparation of Chrysanthemum Plant Expressing DREB1a Gene

The rd29A-DREB1A expression vector described in Kasuga et al's report [Nature Biotech., 17 (1999) 287-291] is shown in FIG. 1. This vector was introduced into the Agrobacterium tumefaciens AGL0 strain by the electroporation method. The Agrobacterium tumefaciens AGL0 strain containing rd29A-DREB1A was inoculated into 3 ml of the following YEB-Km medium. After 16 hours of culture at 28° C. in the dark, cells were collected by centrifugation, and were then suspended in 10 ml of the following medium for infection. The suspension was used as a solution for infection. The medium compositions of the YEB-Km medium and the medium for infection are as follows.

YEB-Km medium: 5 g/l beef extract, 1 g/l yeast extract, 5 g/l peptone, 5 g/l sucrose, 2 mM magnesium sulfate (pH 7.2), and 50 mg/l kanamycin (Km)

Medium for infection: inorganic salt and vitamins in a half concentration of a MS [Murashige & Skoog, Physiol. Plant., 15 (1962) 473-497] medium, 15 g/l sucrose, 10 g/l glucose, and 10 mM MES (pH 5.4)

The leaves of germ-free Lineker plants, Chrysanthemum morifolium cv. Lineker or Dendranthema grandiflorum cv. Lineker, which were Chrysanthemum cultivars, were cut 5 to 7 mm square, and then immersed for 10 minutes in the solution for infection with the agrobacteria, into which the rd29A-DREB1A expression vector had been introduced. After excessive solution for infection had been wiped off on filter paper, transplantation into the following co-culture medium was performed, followed by culture at 25° C. in the dark. After 3 days of culture, cultured cells were transplanted onto the following selection medium, and then cultured for 3 weeks, thereby obtaining Km-resistant calli. Culture was conducted on the selection medium under conditions of 25° C., 16 hours of illumination (light density: 32 μE/m²s)/8 hours of no illumination.

Co-culture medium: MS medium with inorganic salt and vitamins, 30 g/l sucrose, 1 mg/l naphthalenacetic acid, 2 mg/l benzyladenine, 8 g/l agar, 5 mM MES (pH 5.8), and 200 μM acetosyringone

Selection medium: MS medium with inorganic salt and vitamins, 30 g/l sucrose, 1 mg/i naphthalenacetic acid, 2 mg/i benzyladenine, 8 g/l agar, 5 mM MES (pH 5.8), 25 mg/l kanamycin (Km), and 300 mg/i cefotaxime

Plants were regenerated from the obtained Km-resistant calli in the selection media containing Km. Furthermore, the plants were grown on media for promoting rooting that had been prepared by removing plant-growth-regulating substances (naphthalenacetic acid and benzyladenine) from the selection media in order to promote rooting.

Individual plants containing DREB gene were detected from the plants that had grown by performing PCR, and then it was confirmed that the plants that had regenerated were transformants. As primers for specifically amplifying a characteristic sequence of DREB gene, GAGTCTTCGGTTTCCTCA (SEQ ID NO: 29) and CGATACGTCGTCATCATC (SEQ ID NO: 30) were used. PCR reaction was performed under conditions of heating at 94° C. for 5 minutes; 30 cycles of 94° C. (30 seconds), −55° C. (1 minute), and −72° C. (1 minute); and was finally conducted reaction at 72° C. for 10 minutes. In this reaction, Taq polymerase (manufactured by TAKARA SHUZO) was used as an enzyme.

Thus, 13 lines of chrysanthemum having the gene introduced therein were obtained.

### EXAMPLE 2

### Salinity Tolerance Test

The apical buds that had developed 2 to 3 leaves of all the Lineker non-transformants and the Lineker transformants obtained in Example 1 were placed on the following growth media (in vitro) variously supplemented with 0.1, 0.2, and 0.4 M NaCl. Two weeks later, rooting was observed. With 0.2 M NaCl, rooting became unobservable in those buds to which no rd29A-DREB1A gene had been introduced, but rooting was observed in all of buds to which DREB gene had been introduced, excluding a line 14. Even with 0.4 M NaCl, rooting was observed in a line 9. The results for the non-transformants, and lines 9 and 10, are shown in Table 1.

Growth medium: MS medium with inorganic salt and vitamins, 30 g/l sucrose, and 5 mM MES (pH 5.8)

TABLE 1
Salt tolerance test

Added salt concentration (M)

| Line No. | 0 | 0.1 | 0.2 | 0.4 |
|---|---|---|---|---|
| 9 | + | + | + | + |
| 10 | + | + | + | − |
| Non-transformant | + | + | − | − |

### EXAMPLE 3

### Propagation Using Scions and the Following Growth Test

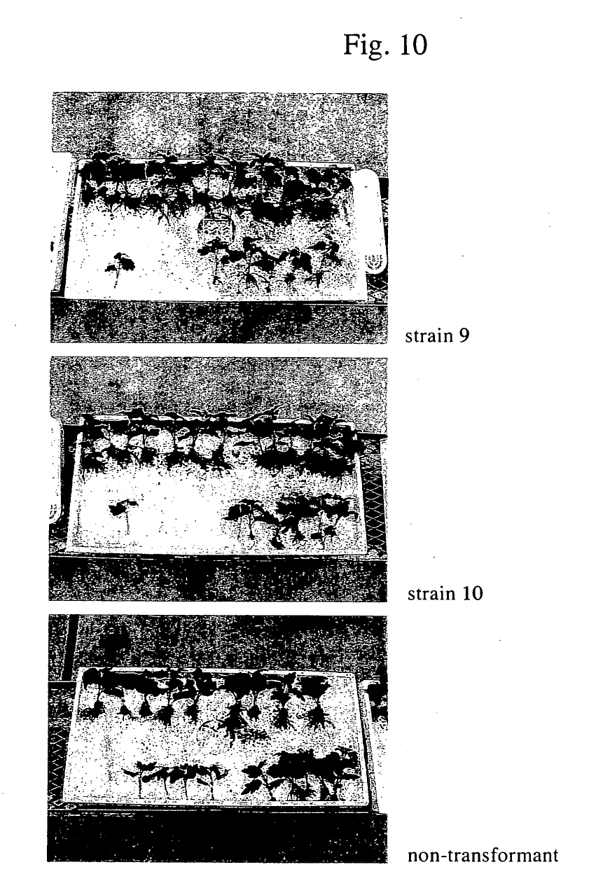

The Lineker non-transformants and lines 9 and 10 of the Lineker transformants obtained in Example 1 were acclimatized in a greenhouse, thereby producing mother plants to obtain scions. Twenty scions were obtained from each line, planted in sufficiently-moistened soil for rooting (Akadama soil: Kanuma soil=1:1), covered with moisture-retaining covers having air permeability, and then cultivated within a greenhouse. Twenty-one days later, plants were harvested so as not to damage the roots from the soil for rooting, and then rooting conditions were observed. The plants were classified in descending order from high to low rooting levels (high, moderate, low, and none (no rooting was observed)), and the number of scions was recorded. The results are shown Table 2 below and in FIG. 10. Surprisingly, rooting ability was significantly improved in lines 9 and 10 to which the rd29A-DREB1A gene had been introduced, compared with that of the Lineker non-transformants.

TABLE 2
Rooting ability test upon scion production

| | Rooting conditions (number of scions) | | | | |
|---|---|---|---|---|---|
| Line No. | High | Moderate | Low | None | Total |
| 9 | 4 | 10 | 5 | 1 | 20 |
| 10 | 6 | 7 | 6 | 1 | 20 |
| Non-transformant | 1 | 8 | 7 | 4 | 20 |

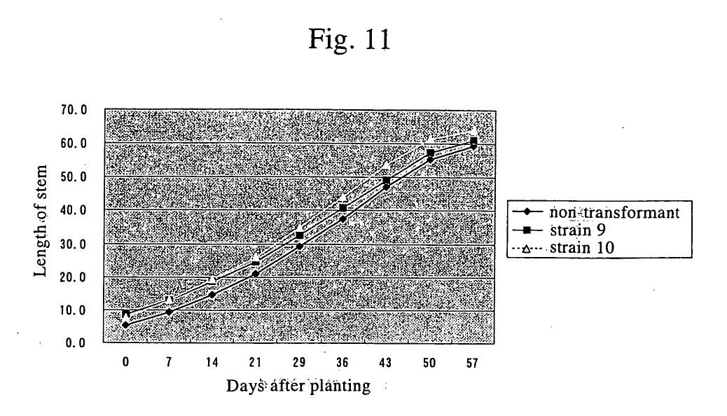

Moreover, 18 to 20 scions were separately obtained by a method similar to the above method. Ten scions showing good rooting (high and moderate according to the above classification) were selected from the scions, and then planted in vinyl pots. Stem length was measured and recorded to study the following growth, and the results are shown in FIG. 11. As shown this figure, compared with the Lineker non-transformants, lines 9 and 10 to which rd29A-DREB1A gene had been introduced showed not only good rooting ability, but also good growth thereafter.

### EXAMPLE 4

### Vase Life Test

Ten individual plants of the Lineker non-transformants and the same of the lines 9 and 10 of the Lineker transformants obtained in Example 3 were then cultivated with long-day conditions (a light period of 18 hours and a dark period of 6 hours) for 4 weeks, and then cultivated with short-day conditions (a light period of 10 hours and a dark period of 14 hours) to cause them to flower. After they had developed 4 to 5 flowers on top, the above ground portions were cut. Cut flowers were arranged in buckets containing tap water, and then stored in a cool and dark place for 2 hours and 30 minutes. Subsequently, the cut flowers were allowed to stand in corrugated cardboard containers for delivery for 17 hours at room temperature, and then arranged in tap water. Vase life test was then conducted. Under conditions employed herein, the cut flowers were allowed to stand in a place where indoor fluorescent lamps were kept on for 11 hours and 30 minutes, while exchanging tap water used for arranging the cut flowers every 2 to 3 days.

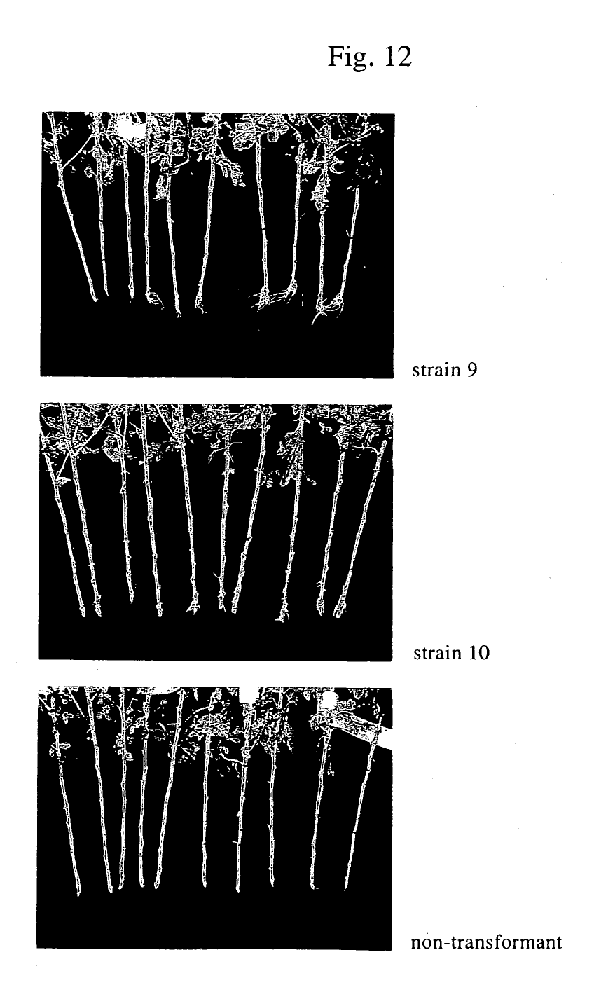

Approximately 2 weeks after the start of the vase life test, no differences were found between the Lineker non-transformants and the Lineker transformants. However, 16 days later, rooting from stems several centimeters above the cut end was observed in both transformed lines. Twenty-two days later, rooting could be observed in most plants of the transformed lines, whereas no rooting was observed in the non-transformed lines (FIG. 12 and Table 3). Thereafter, compared with plants showing no rooting, it was observed that plants showing rooting were clearly exhibiting good plant conditions (in terms of vigor and wilting in flowers, stems, and leaves) and had prolonged vase life (Table 4).

TABLE 3
Rooting conditions upon vase life test

| | Number of plants showing rooting | | | |
|---|---|---|---|---|
| | Days after the start of test (day) | | | |
| Line No. | 1 | 16 | 22 | Total |
| 9 | 0 | 8 | 8 | 10 |
| 10 | 0 | 2 | 9 | 10 |
| Non-transformant | 0 | 0 | 0 | 10 |

TABLE 4
Cut flower conditions on day 22 after the start of vase life test
(Number of plants)

| | Flower conditions *1 | | Stem/Leaf conditions *2 | | |
|---|---|---|---|---|---|
| Line No. | Good | Poor | Good | Poor | Total |
| 9 | 8 | 2 | 8 | 2 | 10 |
| 10 | 9 | 1 | 9 | 1 | 10 |
| Non- | 0 | 10 | 0 | 10 | 10 |
| transformant | | | | | |

All the plants exhibiting good conditions had rooted.

Sequence Listing Free Text
29: primer
30: primer

What is claimed is:

1. A method of producing a transformed plant having improved rooting efficiency and/or prolonged vase life, comprising transforming a plant using a gene wherein a DNA encoding a protein that binds to a stress-responsive element contained in a stress-responsive promoter and regulates the transcription of a gene located downstream of the element is ligated downstream of the stress-responsive promoter.

2. The method of producing a transformed plant of claim 1, wherein the stress-responsive promoter is at least one promoter selected from the group consisting of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter.

3. The method of producing a transformed plant of claim 1, wherein the DNA encoding a protein that binds to a stress-responsive element and regulates the transcription of a gene located downstream of the element is at least one gene selected from the group consisting of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene.

4. The method of producing a transformed plant of claim 1, wherein the DNA encoding a protein that binds to a stress-responsive element and regulates the transcription of a gene located downstream of the element is at least one DNA selected from the group consisting of:
(a) a DNA comprising a nucleotide sequence derived from the nucleotide sequence of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene by deletion, substitution, addition, or insertion of one or several nucleotides, and encoding a protein having activity to bind to a stress-responsive element so as to regulate the transcription of a gene located downstream of the element;
(b) a DNA comprising a nucleotide sequence having at least 80% or more homology with the nucleotide sequence of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element and regulate the transcription of a gene located downstream of the element; and
(c) a DNA hybridizing under stringent conditions to a DNA complementary to a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element and regulate the transcription of a gene located downstream of the element.

5. The method of producing a transformed plant of claim 1, wherein the DNA of a stress-responsive promoter is at least one DNA selected from the group consisting of:
(a) a DNA comprising a nucleotide sequence derived from the nucleotide sequence of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter by deletion, substitution, addition, or insertion of one or several nucleotides, and having activity as the DNA of the stress-responsive promoter;
(b) a DNA comprising a nucleotide sequence having at least 80% or more homology with the nucleotide sequence of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter, and having activity as the DNA of the stress-responsive promoter; and
(c) a DNA hybridizing under stringent conditions to a DNA complementary to a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter, and having activity as the DNA of the stress-responsive promoter.

6. A transformed plant having improved rooting efficiency and/or prolonged vase life, comprising a gene wherein a DNA encoding a protein that binds to a stress-responsive element contained in a stress-responsive promoter and regulates the transcription of a gene located downstream of the element is ligated downstream of the stress-responsive promoter.

7. The transformed plant of claim 6, wherein the stress-responsive promoter is at least one promoter selected from the group consisting of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter.

8. The transformed plant of claim 6, wherein the DNA encoding a protein that binds to a stress-responsive element so as to regulate the transcription of a gene located downstream of the element is at least one gene selected from the group consisting of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene.

9. The transformed plant of claim 6, wherein the DNA encoding a protein that binds to a stress-responsive element and regulates the transcription of a gene located downstream of the element is at least one DNA selected from the group consisting of:
(a) a DNA comprising a nucleotide sequence derived from the nucleotide sequence of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene by deletion, substitution, addition, or insertion of one or several nucleotides, and encoding a protein having activity to bind to a stress-responsive element and regulate the transcription of a gene located downstream of the element;
(b) a DNA comprising a nucleotide sequence having at least 80% or more homology with the nucleotide sequence of a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element and regulate the transcription of a gene located downstream of the element; and
(c) a DNA hybridizing under stringent conditions to a DNA complementary to a DNA of at least one of DREB1A gene, DREB1B gene, DREB1C gene, DREB1D gene, DREB1E gene, DREB1F gene, DREB2A gene, DREB2B gene, DREB2C gene, DREB2D gene, DREB2E gene, DREB2F gene, DREB2G gene, and DREB2H gene, and encoding a protein having activity to bind to a stress-responsive element and regulate the transcription of a gene located downstream of the element.

10. The transformed plant of claim 6, wherein the DNA of a stress-responsive promoter is at least one DNA selected from the group consisting of:
(a) a DNA comprising a nucleotide sequence derived from the nucleotide sequence of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter by deletion, substitution, addition, or insertion of one or several nucleotides, and having activity as the DNA of the stress-responsive promoter;
(b) a DNA comprising a nucleotide sequence having at least 80% or more homology with the nucleotide sequence of a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin gene promoter, and having activity as the DNA of the stress-responsive promoter; and
(c) a DNA hybridizing under stringent conditions to a DNA complementary to a DNA of at least one of rd29A gene promoter, rd29B gene promoter, rd17 gene promoter, rd22 gene promoter, DREB1A gene promoter, cor6.6 gene promoter, cor15a gene promoter, erd1 gene promoter, and kin1 gene promoter, and having activity as the DNA of the stress-responsive promoter.


SEQUENCE LISTING (30 sequences)

SEQ ID NO: 1 - DNA, 933 bp, Arabidopsis thaliana
  CDS (119)..(766)
cctgaactagaacagaaagagagagaaactattatttcagcaaaccataccaacaaaaaagacagagatcttttagttaccttatccagtttcttgaaacagagtactcttctgatcaatgaactcattttctgctttttctgaaatgtttggctccgattacgagtcttcggtttcctcaggcggtgattatattccgacgcttgcgagcagctgccccaagaaaccggcgggtcgtaagaagtttcgtgagactcgtcacccaatatacagaggagttcgtcggagaaactccggtaagtgggtttgtgaggttagagaaccaaacaagaaaacaaggatttggctcggaacatttcaaaccgctgagatggcagctcgagctcacgacgttgccgctttagcccttcgtggccgatcagcctgtctcaatttcgctgactcggcttggagactccgaatcccggaatcaacttgcgctaaggacatccaaaaggcggcggctgaagctgcgttggcgtttcaggatgagatgtgtgatgcgacgacggatcatggcttcgacatggaggagacgttggtggaggctatttacacggcggaacagagcgaaaatgcgttttatatgcacgatgaggcgatgtttgagatgccgagtttgttggctaatatggcagaagggatgcttttgccgcttccgtccgtacagtggaatcataatcatgaagtcgacggcgatgatgacgacgtatcgttatggagttattaaaactcagattattatttccatttttagtacgatactttttattttattattatttttagatccttttttagaatggaatcttcattatgtttgtaaaactgagaaacgagtgtaaattaaattgattcagtttcagtataaaaaaaaaaaaaaaaaaaaaaaaa

SEQ ID NO: 2 - protein, 216 aa, Arabidopsis thaliana
MNSFSAFSEMFGSDYESSVSSGGDYIPTLASSCPKKPAGRKKFRETRHPIYRGVRRRNSGKWVCEVREPNKKTRIWLGTFQTAEMAARAHDVAALALRGRSACLNFADSAWRLRIPESTCAKDIQKAAAEAALAFQDEMCDATTDHGFDMEETLVEAIYTAEQSENAFYMHDEAMFEMPSLLANMAEGMLLPLPSVQWNHNHEVDGDDDDVSLWSY

SEQ ID NO: 3 - DNA, 1437 bp, Arabidopsis thaliana
  CDS (167)..(1171)
gctgtctgataaaaagaagaggaaaactcgaaaaagctacacacaagaagaagaagaaaagatacgagcaagaagactaaacacgaaagcgatttatcaactcgaaggaagagactttgattttcaaatttcgtcccctatagattgtgttgtttctgggaaggagatggcagtttatgatcagagtggagatagaaacagaacacaaattgatacatcgaggaaaaggaaatctagaagtagaggtgacggtactactgtggctgagagattaaagagatggaaagagtataacgagaccgtagaagaagtttctaccaagaagaggaaagtacctgcgaaagggtcgaagaagggttgtatgaaaggtaaaggaggaccagagaatagccgatgtagtttcagaggagttaggcaaaggatttggggtaaatgggttgctgagatcagagagcctaatcgaggtagcaggctttggcttggtactttccctactgctcaagaagctgcttctgcttatgatgaggctgctaaagctatgtatggtcctttggctcgtcttaatttccctcggtctgatgcgtctgaggttacgagtacctcaagtcagtctgaggtgtgtactgttgagactcctggttgtgttcatgtgaaaacagaggatccagattgtgaatctaaacccttctccggtggagtggagccgatgtattgtctggagaatggtgcggaagagatgaagagaggtgttaaagcggataagcattggctgagcgagtttgaacataactattggagtgatattctgaaagagaaagagaaacagaaggagcaagggattgtagaaacctgtcagcaacaacagcaggattcgctatctgttgcagactatggttggcccaatgatgtggatcagagtcacttggattcttcagacatgtttgatgtcgatgagcttctacgtgacctaaatggcgacgatgtgtttgcaggcttaaatcaggaccggtacccggggaacagtgttgccaacggttcatacaggcccgagagtcaacaaagtggttttgatccgctacaaagcctcaactacggaatacctccgtttcagctcgagggaaaggatggtaatggattcttcgacgacttgagttacttggatctggagaactaaacaaaacaatatgaagctttttggatttgatatttgccttaatcccacaacgactgttgattctctatccgagttttagtgatatagagaactacagaacacgttttttcttgttataaaggtgaactgtatatatcgaaacagtgatatgacaatagagaagacaactatagtttgttagtctgcttctcttaagttgttctttagatatgttttatgttttgtaacaacaggaatgaataatacacacttgtaaaaaaaaa

SEQ ID NO: 4 - protein, 335 aa, Arabidopsis thaliana
MAVYDQSGDRNRTQIDTSRKRKSRSRGDGTTVAERLKRWKEYNETVEEVSTKKRKVPAKGSKKGCMKGKGGPENSRCSFRGVRQRIWGKWVAEIREPNRGSRLWLGTFPTAQEAASAYDEAAKAMYGPLARLNFPRSDASEVTSTSSQSEVCTVETPGCVHVKTEDPDCESKPFSGGVEPMYCLENGAEEMKRGVKADKHWLSEFEHNYWSDILKEKEKQKEQGIVETCQQQQQDSLSVADYGWPNDVDQSHLDSSDMFDVDELLRDLNGDDVFAGLNQDRYPGNSVANGSYRPESQQSGFDPLQSLNYGIPPFQLEGKDGNGFFDDLSYLDLEN

SEQ ID NO: 5 - DNA, 937 bp, Arabidopsis thaliana
  CDS (164)..(802)
cttgaaaaagaatctacctgaaaagaaaaaaaagagagagagatataaatagctttaccaagacagatatactatcttttattaatccaaaaagactgagaactctagtaactacgtactacttaaaccttatccagtttcttgaaacagagtactctgatcaatgaactcattttcagctttttctgaaatgtttggctccgattacgagcctcaaggcggagattattgtccgacgttggccacgagttgtccgaagaaaccggcgggccgtaagaagtttcgtgagactcgtcacccaatttacagaggagttcgtcaaagaaactccggtaagtgggtttctgaagtgagagagccaaacaagaaaaccaggatttggctcgggactttccaaaccgctgagatggcagctcgtgctcacgacgtcgctgcattagccctccgtggccgatcagcatgtctcaacttcgctgactcggcttggcggctacgaatcccggagtcaacatgcgccaaggatatccaaaaagcggctgctgaagcggcgttggcttttcaagatgagacgtgtgatacgacgaccacgaatcatggcctggacatggaggagacgatggtggaagctatttatacaccggaacagagcgaaggtgcgttttatatggatgaggagacaatgtttgggatgccgactttgttggataatatggctgaaggcatgcttttaccgccgccgtctgttcaatggaatcataattatgacggcgaaggagatggtgacgtgtcgctttggagttactaatattcgatagtcgtttccatttttgtactatagtttgaaaatattctagttcctttttttagaatggttccttcattttattttattttattgttgtagaaacgagtggaaaataattcaatacaaaaaaaa

SEQ ID NO: 6 - protein, 213 aa, Arabidopsis thaliana
MNSFSAFSEMFGSDYEPQGGDYCPTLATSCPKKPAGRKKFRETRHPIYRGVRQRNSGKWVSEVREPNKKTRIWLGTFQTAEMAARAHDVAALALRGRSACLNFADSAWRLRIPESTCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

SEQ ID NO: 7 - DNA, 944 bp, Arabidopsis thaliana
  CDS (135)..(782)
cctgaattagaaaagaaagatagatagagaaataaatattttatcataccatacaaaaaaagacagagatcttctacttactctactctcataaaccttatccagtttcttgaaacagagtactcttctgatcaatgaactcattttctgccttttctgaaatgtttggctccgattacgagtctccggtttcctcaggcggtgattacagtccgaagcttgccacgagctgccccaagaaaccagcgggaaggaagaagtttcgtgagactcgtcacccaatttacagaggagttcgtcaaagaaactccggtaagtgggtgtgtgagttgagagagccaaacaagaaaacgaggatttggctcgggactttccaaaccgctgagatggcagctcgtgctcacgacgtcgccgccatagctctccgtggcagatctgcctgtctcaatttcgctgactcggcttggcggctacgaatcccggaatcaacctgtgccaaggaaatccaaaaggcggcggctgaagccgcgttgaattttcaagatgagatgtgtcatatgacgacggatgctcatggtcttgacatggaggagaccttggtggaggctatttatacgccggaacagagccaagatgcgttttatatggatgaagaggcgatgttggggatgtctagtttgttggataacatggccgaagggatgcttttaccgtcgccgtcggttcaatggaactataattttgatgtcgagggagatgatgacgtgtccttatggagctattaaaattcgatttttatttccatttttggtattatagctttttatacatttgatccttttttagaatggatcttcttctttttttggttgtgagaaacgaatgtaaatggtaaaagttgttgtcaaatgcaaatgtttttgagtgcagaatatataatcttt

SEQ ID NO: 8 - protein, 216 aa, Arabidopsis thaliana
MNSFSAFSEMFGSDYESPVSSGGDYSPKLATSCPKKPAGRKKFRETRHPIYRGVRQRNSGKWVCELREPNKKTRIWLGTFQTAEMAARAHDVAAIALRGRSACLNFADSAWRLRIPESTCAKEIQKAAAEAALNFQDEMCHMTTDAHGLDMEETLVEAIYTPEQSQDAFYMDEEAMLGMSSLLDNMAEGMLLPSPSVQWNYNFDVEGDDDVSLWSY

SEQ ID NO: 9 - DNA, 1513 bp, Arabidopsis thaliana
  CDS (183)..(1172)
  misc_feature (1443), (1444), (1447), (1450), (1459), (1472), (1495),(1508), (1510): n is A, C, G or T
gagacgctagaaagaacgcgaaagcttgcgaagaagatttgcttttgatcgacttaacacgaacaacaaacaacatctgcgtgataaagaagagatttttgcctaaataaagaagagattcgactctaatcctggagttatcattcacgatagattcttagattgcgactataaagaagaagatggctgtatatgaacaaaccggaaccgagcagccgaagaaaaggaaatctagggctcgagcaggtggtttaacggtggctgataggctaaagaagtggaaagagtacaacgagattgttgaagcttcggctgttaaagaaggagagaaaccgaaacgcaaagttcctgcgaaagggtcgaagaaaggttgtatgaagggtaaaggaggaccagataattctcactgtagttttagaggagttagacaaaggatttggggtaaatgggttgcagagattcgagaaccgaaaataggaactagactttggcttggtacttttcctaccgcggaaaaagctgcttccgcttatgatgaagcggctaccgctatgtacggttcattggctcgtcttaacttccctcagtctgttgggtctgagtttactagtacgtctagtcaatctgaggtgtgtacggttgaaaataaggcggttgtttgtggtgatgtttgtgtgaagcatgaagatactgattgtgaatctaatccatttagtcagattttagatgttagagaagagtcttgtggaaccaggccggacagttgcacggttggacatcaagatatgaattcttcgctgaattacgatttgctgttagagtttgagcagcagtattggggccaagttttgcaggagaaagagaaaccgaagcaggaagaagaggagatacagcaacagcaacaggaacagcaacagcaacagctgcaaccggatttgcttactgttgcagattacggttggccttggtctaatgatattgtaaatgatcagacttcttgggatcctaatgagtgctttgatattaatgaactccttggagatttgaatgaacctggtccccatcagagccaagaccaaaaccacgtaaattctggtagttatgatttgcatccgcttcatctcgagccacacgatggtcacgagttcaatggtttgagttctctggatatttgagagttctgaggcaatggtcctacaagactacaacataatctttggattgatcataggagaaacaagaaataggtgttaatgatctgattcacaatgaaaaaatatttaataactctatagtttttgttctttccttggatcatgaactgttgcttctcatctattgagttaatatagcgaatagcagagtttctctctttcttctctttgtagaaaaaaaaaaaaaaaaaaaaaaaaaaaaaaaayhsakmabgcarsrcsdvsnaanntrnatnarsarchcntrragrctrascncsrcaswashtskbabarakaantamaysakmasrngngac

SEQ ID NO: 10 - protein, 330 aa, Arabidopsis thaliana
MAVYEQTGTEQPKKRKSRARAGGLTVADRLKKWKEYNEIVEASAVKEGEKPKRKVPAKGSKKGCMKGKGGPDNSHCSFRGVRQRIWGKWVAEIREPKIGTRLWLGTFPTAEKAASAYDEAATAMYGSLARLNFPQSVGSEFTSTSSQSEVCTVENKAVVCGDVCVKHEDTDCESNPFSQILDVREESCGTRPDSCTVGHQDMNSSLNYDLLLEFEQQYWGQVLQEKEKPKQEEEEIQQQQQEQQQQQLQPDLLTVADYGWPWSNDIVNDQTSWDPNECFDINELLGDLNEPGPHQSQDQNHVNSGSYDLHPLHLEPHDGHEFNGLSSLDI

SEQ ID NO: 11 - DNA, 675 bp, Arabidopsis thaliana
atgaatccattttactctacattcccagactcgtttctctcaatctccgatcatagatctccggtttcagacagtagtgagtgttcaccaaagttagcttcaagttgtccaaagaaacgagctgggaggaagaagtttcgtgagacacgtcatccgatttacagaggagttcgtcagaggaattctggtaaatgggtttgtgaagttagagagcctaataagaaatctaggatttggttaggtacttttccgacggttgaaatggctgctcgtgctcatgatgttgctgctttagctcttcgtggtcgctctgcttgtctcaatttcgctgattctgcttggcggcttcgtattcctgagactacttgtcctaaggagattcagaaagctgcgtctgaagctgcaatggcgtttcagaatgagactacgacggagggatctaaaactgcggcggaggcagaggaggcggcaggggagggggtgagggagggggagaggagggcggaggagcagaatggtggtgtgttttatatggatgatgaggcgcttttggggatgcccaacttttttgagaatatggcggaggggatgcttttgccgccgccggaagttggctggaatcataacgactttgacggagtgggtgacgtgtcactctggagttttgacgagtaa

SEQ ID NO: 12 - protein, 224 aa, Arabidopsis thaliana
MNPFYSTFPDSFLSISDHRSPVSDSSECSPKLASSCPKKRAGRKKFRETRHPIYRGVRQRNSGKWVCEVREPNKKSRIWLGTFPTVEMAARAHDVAALALRGRSACLNFADSAWRLRIPETTCPKEIQKAASEAAMAFQNETTTEGSKTAAEAEEAAGEGVREGERRAEEQNGGVFYMDDEALLGMPNFFENMAEGMLLPPPEVGWNHNDFDGVGDVSLWSFDE

SEQ ID NO: 13 - DNA, 546 bp, Arabidopsis thaliana
atggaaaacgacgatatcaccgtggcggagatgaagccaaagaagcgtgctggacggaggattttcaaggagacacgtcacccaatctacagaggcgtgcggcgtagggacggcgacaaatgggtatgcgaagtccgtgaaccgattcatcagcgtcgagtctggctcggaacttatccgacggcagatatggccgcacgtgctcacgacgtggcggttcttgctctgcgcgggagatccgcgtgtttgaatttctccgattctgcttggaggttgccggtgccggcatccactgatccggacacgatcaggcgcacggcggccgaagcagcggagatgttcaggccgccggagtttagtacaggaattacggttttaccctcagccagtgagtttgacacgtcggatgaaggagtcgctggaatgatgatgaggctcgcggaggagccgttgatgtcgccgccaagatcgtacattgatatgaatacgagtgtgtacgtggacgaagaaatgtgttacgaagatttgtcactttggagttactaa

SEQ ID NO: 14 - protein, 181 aa, Arabidopsis thaliana
MENDDITVAEMKPKKRAGRRIFKETRHPIYRGVRRRDGDKWVCEVREPIHQRRVWLGTYPTADMAARAHDVAVLALRGRSACLNFSDSAWRLPVPASTDPDTIRRTAAEAAEMFRPPEFSTGITVLPSASEFDTSDEGVAGMMMRLAEEPLMSPPRSYIDMNTSVYVDEEMCYEDLSLWSY

SEQ ID NO: 15 - DNA, 630 bp, Arabidopsis thaliana
atgaataatgatgatattattctggcggagatgaggcctaagaagcgtgcgggaaggagagtgtttaaggagacacgtcacccagtttacagaggcataaggcggaggaacggtgacaaatgggtctgcgaagtcagagaaccgacgcaccaacgccgcatttggctcgggacttatcccacagcagatatggcagcgcgtgcacacgacgtggcggttttagctctgcgtgggagatccgcatgtttgaatttcgccgactccgcttggcggcttccggtgccggaatccaatgatccggatgtgataagaagagttgcggcggaagctgcggagatgtttaggccggtggatttagaaagtggaattacggttttgccttgtgcgggagatgatgtggatttgggttttggttcgggttccggctctggttcgggatcggaggagaggaattcttcttcgtatggatttggagactacgaagaagtctcaacgacgatgatgagactcgcggaggggccactaatgtcgccgccgcgatcgtatatggaagacatgactcctactaatgtttacacggaagaagagatgtgttatgaagatatgtcattgtggagttacagatattaa

SEQ ID NO: 16 - protein, 209 aa, Arabidopsis thaliana
MNNDDIILAEMRPKKRAGRRVFKETRHPVYRGIRRRNGDKWVCEVREPTHQRRIWLGTYPTADMAARAHDVAVLALRGRSACLNFADSAWRLPVPESNDPDVIRRVAAEAAEMFRPVDLESGITVLPCAGDDVDLGFGSGSGSGSGSEERNSSSYGFGDYEEVSTTMMRLAEGPLMSPPRSYMEDMTPTNVYTEEEMCYEDMSLWSYRY

SEQ ID NO: 17 - DNA, 1026 bp, Arabidopsis thaliana
atgccgtcggagattgttgacaggaaaaggaagtctcgtggaacacgagatgtagctgagattctaaggcaatggagagagtacaatgagcagattgaggcagaatcttgtatcgatggtggtggtccaaaatcaatccgaaagcctcctccaaaaggttcgaggaagggttgtatgaaaggtaaaggtggacctgaaaacgggatttgtgactatagaggagttagacagaggagatggggtaaatgggttgctgagatccgtgagccagacggaggtgctaggttgtggctcggtactttctccagttcatatgaagctgcattggcttatgacgaggcggccaaagctatatatggtcagtctgccagactcaatcttcccgagatcacaaatcgctcttcttcgactgctgccactgccactgtgtcaggctcggttactgcattttctgatgaatctgaagtttgtgcacgtgaggatacaaatgcaagttcaggttttggtcaggtgaaactagaggattgtagcgatgaatatgttctcttagatagttctcagtgtattaaagaggagctgaaaggaaaagaggaagtgagggaagaacataacttggctgttggttttggaattggacaggactcgaaaagggagactttggatgcttggttgatgggaaatggcaatgaacaagaaccattggagtttggtgtggatgaaacgtttgatattaatgagctattgggtatattaaacgacaacaatgtgtctggtcaagagacaatgcagtatcaagtggatagacacccaaatttcagttaccaaacgcagtttccaaattctaacttgctcgggagcctcaaccctatggagattgctcaaccaggagttgattatggatgtccttatgtgcagcccagtgatatggagaactatggtattgatttagaccatcgcaggttcaatgatcttgacatacaggacttggattttggaggagacaaagatgttcatggatctacataa

SEQ ID NO: 18 - protein, 341 aa, Arabidopsis thaliana
MPSEIVDRKRKSRGTRDVAEILRQWREYNEQIEAESCIDGGGPKSIRKPPPKGSRKGCMKGKGGPENGICDYRGVRQRRWGKWVAEIREPDGGARLWLGTFSSSYEAALAYDEAAKAIYGQSARLNLPEITNRSSSTAATATVSGSVTAFSDESEVCAREDTNASSGFGQVKLEDCSDEYVLLDSSQCIKEELKGKEEVREEHNLAVGFGIGQDSKRETLDAWLMGNGNEQEPLEFGVDETFDINELLGILNDNNVSGQETMQYQVDRHPNFSYQTQFPNSNLLGSLNPMEIAQPGVDYGCPYVQPSDMENYGIDLDHRRFNDLDIQDLDFGGDKDVHGST

SEQ ID NO: 19 - DNA, 621 bp, Arabidopsis thaliana
atgtcatccatagagccaaaagtaatgatggttggtgctaataagaaacaacgaaccgtccaagctagttcgaggaaaggttgtatgagaggaaaaggtggacccgataacgcgtcttgcacttacaaaggtgttagacaacgcacttggggcaaatgggtcgctgagatccgcgagcctaaccgaggagctcgtctttggctcggtaccttcgacacctcccgtgaagctgccttggcttatgactccgcagctcgtaagctctatgggcctgaggctcatctcaacctccctgagtccttaagaagttaccctaaaacggcgtcgtctccggcgtcccagactacaccaagcagcaacaccggtggaaaaagcagcagcgactctgagtcgccgtgttcatccaacgagatgtcatcatgtggaagagtgacagaggagatatcatgggagcatataaacgtggatttgccggtaatggatgattcttcaatatgggaagaagctacaatgtcgttaggatttccatgggttcatgaaggagataatgatatttctcggtttgatacttgtatttccggtggctattctaattgggattcctttcattccccactttga

SEQ ID NO: 20 - protein, 206 aa, Arabidopsis thaliana
MSSIEPKVMMVGANKKQRTVQASSRKGCMRGKGGPDNASCTYKGVRQRTWGKWVAEIREPNRGARLWLGTFDTSREAALAYDSAARKLYGPEAHLNLPESLRSYPKTASSPASQTTPSSNTGGKSSSDSESPCSSNEMSSCGRVTEEISWEHINVDLPVMDDSSIWEEATMSLGFPWVHEGDNDISRFDTCISGGYSNWDSFHSPL

SEQ ID NO: 21 - DNA, 975 bp, Arabidopsis thaliana
atggaaaaggaagataacggatcgaaacagagctcctctgcttctgttgtatcctcgagaagacgaagaagagtggttgagccagtggaagcgacgttacagagatgggaggaagaaggattggcgagagctcgtagggttcaagccaaaggttcgaagaaaggttgtatgagaggaaaaggtggaccagagaatcctgtttgtcggtttagaggtgttcgacaaagggtttgggggaaatgggttgctgagatacgtgaaccagtgagtcaccgtggtgcaaactctagtcgtagtaaacggctttggcttggcacgtttgctactgcagctgaagctgctttggcttacgacagagctgctagtgtcatgtacggaccctatgccaggttaaatttcccggaagatttgggtgggggaaggaagaaggacgaggaggcggaaagttcgggaggctattggttggaaactaacaaagccggtaatggcgtgattgaaacggaaggtggaaaagactatgtagtctacaatgaagacgctattgagcttggccatgacaagactcagaatcctgacatgtttgatgtcgatgagcttctacgtgacctaaatggcgacgatgtgtttgcaggcatgactgataatgaaatagtgaacccagcagttaaatcaggaccggtacccggggaacagtgttgccaacggttcatacaggcccgagagttgaaatcagaggaaggttacagctatgatcgattcaaattggcaacaaagtggttttgatccgctacaaagcctcaactacggaatacctccgtttcagctcataacggattgttgtataatgaacctcaaagctccagttatcacgagggaaaggatggtaatggattcttcgacgacttgagttacttggatctggagaactaacagggaggtggattcgattcatattttgagtatttcagattctag

SEQ ID NO: 22 - protein, 244 aa, Arabidopsis thaliana
MEKEDNGSKQSSSASVVSSRRRRRVVEPVEATLQRWEEEGLARARRVQAKGSKKGCMRGKGGPENPVCRFRGVRQRVWGKWVAEIREPVSHRGANSSRSKRLWLGTFATAAEAALAYDRAASVMYGPYARLNFPEDLGGGRKKDEEAESSGGYWLETNKAGNGVIETEGGKDYVVYNEDAIELGHDKTQNPMTDNEIVNPAVKSEEGYSYDRFKLDNGLLYNEPQSSSYHQGGGFDSYFEYFRF

SEQ ID NO: 23 - DNA, 834 bp, Arabidopsis thaliana
atggagaaatcatcctcaatgaaacaatggaagaagggtcctgctcggggtaaaggcggtccacaaaacgctctttgtcagtaccgtggagtcaggcaaaggacttggggcaaatgggtggctgagatcagagagcccaagaagagggcaagactttggcttggctctttcgctacagctgaagaagcagctatggcttatgatgaggctgccttgaaactctatgggcacgacgcatacctcaacttacctcatcttcagcggaatacaagaccttctctgagtaactctcagaggttcaaatgggtaccttcaaggaagtttatatctatgtttccttcatgtggtatgctaaacgtgaatgctcagcctagtgttcacataatccagcaaagactagaagaactcaagaaaactggacttttatctcaatcctattcttctagttcttcctccaccgaatcaaaaactaatactagctttcttgatgagaagaccagcaagggagaaacagacaatatgttcgaaggtggtgatcagaagaaaccagagatcgacctgaccgagtttcttcagcaactaggaatcttgaaggatgaaaatgaagcagaaccaagtgaggtagcagagtgtcattcccctccaccatggaacgagcaagaagaaactggaagtcctttcagaactgagaatttcagctgggataccctgatcgagatgccaagaagtgaaaccacaactatgcaatttgactccagcaacttcggaagctatgattttgaggatgatgtatccttcccttccatctgggactactacggaagcttagattga

SEQ ID NO: 24 - protein, 277 aa, Arabidopsis thaliana
MEKSSSMKQWKKGPARGKGGPQNALCQYRGVRQRTWGKWVAEIREPKKRARLWLGSFATAEEAAMAYDEAALKLYGHDAYLNLPHLQRNTRPSLSNSQRFKWVPSRKFISMFPSCGMLNVNAQPSVHIIQQRLEELKKTGLLSQSYSSSSSSTESKTNTSFLDEKTSKGETDNMFEGGDQKKPEIDLTEFLQQLGILKDENEAEPSEVAECHSPPPWNEQEETGSPFRTENFSWDTLIEMPRSETTTMQFDSSNFGSYDFEDDVSFPSIWDYYGSLD

SEQ ID NO: 25 - DNA, 924 bp, Arabidopsis thaliana
atggaagaagagcaacctccggccaagaaacgaaacatggggagatctagaaaaggttgcatgaaaggtaaaggcggtccagagaacgccacgtgtactttccgtggagttaggcaacggacttggggtaaatgggtggctgagatccgtgagcctaaccgtgggactcgtctctggctcggcacgtttaatacctcggtcgaggccgccatggcttacgatgaagccgctaagaaactctatggacacgaggctaaactcaacttggtgcacccacaacaacaacaacaagtagtagtgaacagaaacttgtctttttctggccacgggtcgggttcttgggcttataataagaagctcgatatggttcatgggttggaccttggtctcggccaggcaagttgttcacgaggttcttgctcagagagatcgagttttctacaagaagatgatgatcatagtcataatcgatgttcgtcttcaagtggttcgaatctttgttggttattacctaaacaaagtgattcacaagatcaagagaccgttaatgctacgactagttatggcggtgaaggcggtggtggctctacgttaacgttttcgaccaatttgaaaccaaagaatttgatgagtcagaattatggattatacaatggagcttggtctaggtttcttgtggggcaagaaaagaagacggaacatgacgtgtcatcgtcgtgtggatcgtcggacaacaaggagagtatgttggttcctagttgcggcggagagaggatgcataggccggagttggaagagcgaacaggatatttggaaatggatgatcttttggagattgatgatttaggtttgttgattggcaaaaatggagatttcaagaattggtgttgtgaagagtttcaacatccatggaattggttctga

SEQ ID NO: 26 - protein, 306 aa, Arabidopsis thaliana
EEEQPPAKKRNMGRSRKGCMKGKGGPENATCTFRGVRQRTWGKWVAEIREPNRGTRLWLGTFNTSVEAAMAYDEAAKKLYGHEAKLNLVHPQQQQQVVVNRNLSFSGHGSGSWAYNKKLDMVHGLDLGLGQASCSRGSCSERSSFLQEDDDHSHNRCSSSSGSNLCWLLPKQSDSQDQETVNATTSYGGEGGGGSTLTFSTNLKPKNLMSQNYGLYNGAWSRFLVGQEKKTEHDVSSSCGSSDNKESMLVPSCGGERMHRPELEERTGYLEMDDLLEIDDLGLLIGKNGDFKNWCCEEFQHPWNWF

SEQ ID NO: 27 - DNA, 534 bp, Arabidopsis thaliana
atgcccaggaaacggaagtctcgtggaacacgagatgtagctgagattctaaggaaatggagagagtacaatgagcagaccgaggcagattcttgcatcgatggtggtggttcaaaaccaatccgaaaggctcctccaaaacgttcgaggaagggttgtatgaaaggtaaaggtggacctgaaaatgggatttgtgactatacaggagttagacagaggacatggggtaaatgggttgctgagatccgtgagccaggccgaggtgctaagttatggctcggtactttctctagttcatatgaagctgcattggcttatgatgaggcttccaaagctatttacggtcagtctgcccgactcaatcttccactgctgccactgtgtcaggctcggttactgcattttctgatgaatctgaagtttgtgcacgtgaggatacaaatgcaagatctggttttggtcagatctctaacttctcgcatttccaaaatgttaagtccaataactgcattggttaagttggggcgttactag

SEQ ID NO: 28 - protein, 177 aa, Arabidopsis thaliana
MPRKRKSRGTRDVAEILRKWREYNEQTEADSCIDGGGSKPIRKAPPKRSRKGCMKGKGGPENGICDYTGVRQRTWGKWVAEIREPGRGAKLWLGTFSSSYEAALAYDEASKAIYGQSARLNLPLLPLCQARLLHFLMNLKFVHVRIQMQDLVLVRSLTSRISKMLSPITALVKLGRY

SEQ ID NO: 29 - DNA, 18 bp, Artificial Sequence
  Description of Artificial Sequence: Primer
gagtcttcggtttcctca

SEQ ID NO: 30 - DNA, 18 bp, Artificial Sequence
  Description of Artificial Sequence: Primer
cgatacgtcgtcatcatc